(19) United States
(12) Patent Application Publication
(10) Pub. No.: US 2026/0048147 A1
(43) Pub. Date: Feb. 19, 2026

(54) AAV CAPSID PROTEINS HAVING MUTATIONS IN THE VP1 REGION

(71) Applicant: University of Massachusetts, Westborough, MA (US)

(72) Inventors: Guangping Gao, Worcester, MA (US); Phillip Tai, Worcester, MA (US); Mengtian Cui, Worcester, MA (US)

(73) Assignee: University of Massachusetts, Westborough, MA (US)

(21) Appl. No.: 19/249,079

(22) Filed: Jun. 25, 2025

Related U.S. Application Data
(63) Continuation of application No. PCT/US2024/024867, filed on Apr. 17, 2024.
(60) Provisional application No. 63/496,715, filed on Apr. 18, 2023.

(51) Int. Cl. A61K 48/00; C07K 14/005; C12N 15/86

(52) U.S. Cl. CPC A61K 48/0058; C07K 14/005; C12N 15/86; C12N 2750/14122; C12N 2750/14143; C12N 2830/008

(57) ABSTRACT

Aspects of the disclosure relate to compositions and methods for delivering a transgene (e.g., a transgene encoding one or more gene products) to a target cell. The disclosure is based, in part, on adeno-associated virus (AAV) capsid proteins comprising one or more amino acid substitutions in the VP1 region, and methods of using same for delivery of a transgene.

### RELATED APPLICATION

This application claims the benefit under 35 U.S.C. § 365(c) and § 120 and is a continuation of International Patent Application Number PCT/US2024/024867, filed Apr. 17, 2024, titled “AAV CAPSID PROTEINS HAVING MUTATIONS IN THE VP1 REGION”, which claims priority under 35 U.S.C. § 119(e) to U.S. provisional patent application 63/496,715, filed Apr. 18, 2023, the entire contents of each of which are incorporated by reference herein.

### REFERENCE TO AN ELECTRONIC SEQUENCE LISTING

The contents of the electronic sequence listing (U012070181US01-SEQ-KZM.xml; Size: 64,736 bytes; and Date of Creation: Jun. 24, 2025) is herein incorporated by reference in its entirety.

### BACKGROUND OF INVENTION

Recombinant AAV adeno-associated viruses (rAAVs) are capable of driving stable and sustained transgene expression in target tissues without notable toxicity and host immunogenicity. Thus, rAAVs are promising delivery vehicles for long-term therapeutic gene expression. However, low transduction efficiency and restricted tissue tropisms by currently available rAAV vectors can limit their application as feasible and efficacious therapies. Additionally, faithful clinical translation of leading therapeutic AAV serotypes derived from non-human tissues is a concern. Accordingly, a need remains for new AAV vectors for gene delivery.

### SUMMARY OF INVENTION

Aspects of the disclosure relate to compositions and methods for delivering transgenes (e.g., nucleic acid sequences encoding gene products such as proteins and/or functional RNAs) to a cell or subject. The disclosure is based, in part, on AAV capsid protein variants having one or more amino acid substitutions in the VP1 unique (VP1u) region of the capsid protein. In some embodiments, the one or more substitutions result in the capsid protein onto which the modified VP1u sequence is grafted having improved properties, such as increased transduction efficiency, increased intracellular trafficking (e.g., to cell nuclei), etc., relative to unmodified AAV capsid proteins.

Accordingly, in some aspects, the disclosure provides an isolated capsid protein comprising at least three amino acid mutations within a VP1 unique (VP1u) region of the capsid protein, wherein the at least three amino acid mutations are in positions corresponding to positions 36, 80, and 125 (e.g., E36, D80, and V125) with reference to amino acid position numbering of a wild-type AAV2 capsid protein (e.g., SEQ ID NO: 1).

In some embodiments, an isolated capsid protein is of a serotype selected from AAV1, AAV2, AAV3, AAV4, AAV5, AAV6, AAV7, AAV8, AAV9, AAV10, AAV9.PHPeB, MYOAAV, AAVrh74, AAV2.5T, AAV2/8, AAV2/6, AAVrh32.33, Anc80, NP40, NP59, and LKO3.

In some embodiments, a VP1u region comprises an amino acid sequence that is at least 70% identical (e.g., at least 80%, 85%, 90%, or 95% identical) to the amino acid sequence set forth in SEQ ID NO: 1. In some embodiments, the VP1u region comprises an amino acid sequence that is at least 70% identical to amino acid positions 1-200 of SEQ ID NO: 1.

In some embodiments, at least three amino acid substitutions comprise E36G, D80N, and V125A amino acid substitutions with reference to amino acid position numbering of a wild-type AAV2 capsid protein (e.g., SEQ ID NO: 1).

In some embodiments, the capsid protein comprises a glycine (G) at position 36, an asparagine (N) at position 80, and an alanine (A) at position 125 with reference to amino acid position numbering of a wild-type AAV2 capsid protein.

In some embodiments, the capsid protein comprises the amino acid sequence of any one of SEQ ID NOs: 4 or 14-18.

In some aspects, the disclosure provides a recombinant adeno-associated virus (rAAV) comprising an isolated nucleic acid comprising a transgene encoding a gene product flanked by AAV ITRs; and an isolated capsid protein as described herein.

In some embodiments, a gene product is a protein or interfering nucleic acid. In some embodiments, a protein is a therapeutic protein. In some embodiments, an interfering nucleic acid is selected from a dsRNA, siRNA, miRNA, artificial miRNA (ami-RNA), or RNA aptamer.

In some embodiments, a transgene further comprises a promoter. In some embodiments, a promoter is a constitutive promoter, inducible promoter, or a tissue-specific promoter.

In some embodiments, a transgene further comprises one or more miRNA binding sites.

In some aspects, the disclosure provides a method for delivering a transgene to a target cell of a subject, the method comprising administering to the subject an rAAV as described herein.

In some embodiments, a target cell is an ocular cell, central nervous system (CNS) cell, peripheral nervous system (PNS) cell, liver cell, lung cell, muscle cell, bone cell, pancreas cell, stomach cell, or skin cell.

In some embodiments, a subject is a mammal. In some embodiments, a subject is a human.

In some embodiments, administration comprises injection or topical administration.

In some embodiments, administration results in increased expression of the transgene in the target cell relative to expression of the transgene delivered using an rAAV comprising a wild-type AAV capsid protein.

In some aspects, the disclosure provides a method for delivering a transgene to an ocular cell in a subject, the method comprising administering to the subject a recombinant adeno-associated virus (rAAV) comprising an isolated nucleic acid comprising a transgene encoding one or more gene products flanked by AAV ITRs; and an adeno-associated virus (AAV) capsid protein comprising the amino acid sequence set forth in SEQ ID NO: 4.

In some embodiments, administration comprises intraocular administration, intravenous administration, or topical administration to the eye or eyelid. In some embodiments, intraocular administration comprises intravitreal administration, transscleral administration, subconjunctival administration, retrobulbar administration, intracameral administration, or subretinal administration.

In some embodiments, an ocular cell is an amacrine cell, a bipolar cell, a trabecular meshwork cell, a ciliary body cell, a retinal pigment epithelial cell, a retinal cell, an astrocyte, a pericyte, a Müller cell, a ganglion cell, or a photoreceptor cell.

In some embodiments, a subject is a mammal. In some embodiments, a subject is a human.

In some embodiments, a transgene encoding one or more gene products further comprises a promoter. In some embodiments, a promoter is an eye-specific promoter. In some embodiments, an eye-specific promoter is a retinoschisin proximal promoter, interphotoreceptor retinoid-binding protein enhancer (RS/IRBPa) promoter, rhodopsin kinase (RK) promoter, RPE65 promoter, or human cone opsin promoter.

In some embodiments, one or more gene products comprise a protein or an inhibitory nucleic acid. In some embodiments, a protein comprises an anti-VEGF agent. In some embodiments, an anti-VEGF agent is KH902.

In some aspects, the disclosure provides an isolated AAV capsid protein comprising an amino acid sequence having the sequence as set forth in any one of SEQ ID NOs: 4 or 14-18. In some aspects, the disclosure provides a recombinant AAV (rAAV) comprising having the sequence as set forth in any one of SEQ ID NOs: 4 or 14-18.

### BRIEF DESCRIPTION OF DRAWINGS

FIG. 1 depicts an illustration of the strategy used to screen and identify AAV2 variants in mice and non-human primates.

FIGS. 2A-2C show the transduction of Egfp vectors packaged with AAV2.v149, AAV2.v152, and AAV2.MC1 capsids compared to AAV2 and AAV2.7m8 capsids. FIG. 2A shows fundoscopy images of mouse eyes subjected to intravitreal (IVT) injection of the Egfp vectors at a dose of 1.0E9 or 2.5E8 viral genomes (vg)/eye. FIGS. 2B and 2C show the quantification by ddPCR of Egfp expression in mouse eyes one month post-injection with 2.5E8 vg/eye (FIG. 2B) or 1.0E9 vg/eye (FIG. 2C).

FIG. 3 shows the amino acid alignment of the VP1u region between AAV2, AAV2.v149, and AAV2.v152, highlighting the unique residues among the variants in red. From top to bottom, SEQ ID NOs: 24-26 are shown.

FIG. 4 shows the transduction of specific cell types following transduction of scAAV-Egfp vectors packaged with AAV2, AAV2.7m8, AAV2.v149, AAV2.v152, or AAV2.MC1 capsids. The bar graphs represent the percentage of EGFP+ cells in retinal cells co-stained by cell type-specific antibodies to mark ganglion, amacrine, bipolar, and horizontal cells.

FIGS. 5A-5B show the transduction of HeLa cells with rAAV vectors packaged using AAV2, AAV2.v149, AAV2.v152, or AAV2.MC1 capsids. FIG. 5A shows the quantification of relative luciferase activity of HeLa cultures transduced by ssAAV-Fluc vectors at different MOIs 24 hours post-transduction. FIG. 5B shows representative epifluorescence microscopy images of HeLa cultures transduced by scAAV-Egfp vectors at a MOI of 5E3 genome copies (gc)/cell 36 hours post-transduction.

FIGS. 6A-6B show the kinetics of vector DNA distribution following rAAV transduction. FIG. 6A shows a histogram of vector genomes (Egfp) detected by ddPCR in HeLa cells transduced with rAAV-Egfp vectors packaged with AAV2, AAV2.v149, AAV2.v152, or AAV2.MC1 and incubated on ice for 30 minutes. FIG. 6B shows stacked histograms demonstrating the distribution of vector genomes detected in either nuclear (orange) or cytoplasmic (yellow) fractions of HeLA cells at 4 hours (left) or 6 hours (right) following transduction with rAAV-Egfp vectors packaged with the indicated capsids.

FIG. 7 shows fundoscopy images of mouse eyes transduced with rAAV-Egfp vectors packaged with AAV2.7m8, AAV2.7m8.v149, AAV2.7m8.v152, or AAV2.7m8.MC1 capsids at a dose of 1.0E9 or 2.5E8 vg/eye.

FIGS. 8A-8B show a protein sequence alignment indicating the high level of conservation of VP1u across different AAV serotypes. From top to bottom, SEQ ID NOs: 24-34 are shown.

FIGS. 9A-9E provide graphs showing the packaging efficiency of AAV capsid variants of the disclosure.

FIGS. 10A-10B provide epifluorescence microscopy images (FIG. 10A) and graphs derived from flow cytometry experiments (FIG. 10B) to demonstrate the ability of AAVs comprising AAV capsid variants of the disclosure to transduce mammalian cells in vitro.

FIGS. 11A-11E provide data relating to the characterization of an AAV8 MC1 capsid protein (SEQ ID NO: 14). FIGS. 11A-11B provide epifluorescence microscopy images (FIG. 11A) and graphs derived from flow cytometry experiments (FIG. 11B) to show the ability of this AAV8 MCi capsid protein to transduce mammalian cells in vitro. FIGS. 11C-11E provide epifluorescence images of liver tissues (FIG. 11C), quantification of transgene expression (FIG. 11D), and epifluorescence images of muscle tissues (FIG. 11E) following intravenous administration to mouse subjects.

FIGS. 12A-12C provide data relating to the characterization of an AAV9 MCi capsid protein (SEQ ID NO: 15). FIGS. 12A-12B provide epifluorescence microscopy images (FIG. 12A) and graphs derived from flow cytometry experiments (FIG. 12B) to show the ability of this AAV9 MCi capsid protein to transduce mammalian cells in vitro. FIG. 12C provides quantification of transgene expression following intravenous administration to mouse subjects.

FIGS. 13A-13B provide data relating to the characterization of an AAV1 MCi capsid protein (SEQ ID NO: 16). Epifluorescence microscopy images (FIG. 13A) and graphs derived from flow cytometry experiments (FIG. 13B) are provided to show the ability of this AAV1 MCi capsid protein to transduce mammalian cells in vitro.

FIGS. 14A-14D provide data showing the ability of an AAV2 MCi capsid protein (SEQ ID NO: 4) to deliver a transgene to ocular cells following intravitreal administration to non-human primates (NHPs).

### DETAILED DESCRIPTION OF INVENTION

Aspects of the disclosure relate to compositions and methods for delivering a transgene (e.g., a transgene encoding one or more gene products) to a target cell (e.g., an ocular cell such as an amacrine cell, a bipolar cell, a trabecular meshwork cell, a ciliary body cell, a retinal pigment epithelial cell, a retinal cell, an astrocyte, a pericyte, a Müller cell, a ganglion cell, or a photoreceptor cell). The disclosure is based, in part, on adeno-associated virus (AAV) capsid protein variants comprising a VP1 region that confers enhanced intracellular trafficking of recombinant adeno-associated viruses (rAAVs) and improved rAAV transduction in certain cell types (e.g., non-photoreceptor retinal cells) compared to rAAVs comprising certain wild-type AAV capsid proteins. In some embodiments, recombinant AAVs (rAAVs) comprising the capsid protein variants are more efficiently packaged than rAAVs having certain wild-type AAV capsid proteins. In some embodiments, the rAAVs comprising the capsid protein variants are more efficiently internalized in a target cell than rAAVs having certain wild-type AAV capsid proteins. In some embodiments, the rAAVs comprising the capsid protein variants are more efficiently trafficked within a target cell than rAAVs having certain wild-type AAV capsid proteins. In some embodiments, certain regions of the capsid protein variants (e.g., the VP1u region) may be grafted onto other capsid types to enhance rAAV packaging, internalization, and/or trafficking. Methods of delivering an rAAV comprising the AAV capsid protein variants are also described by the disclosure.

### Capsid Proteins

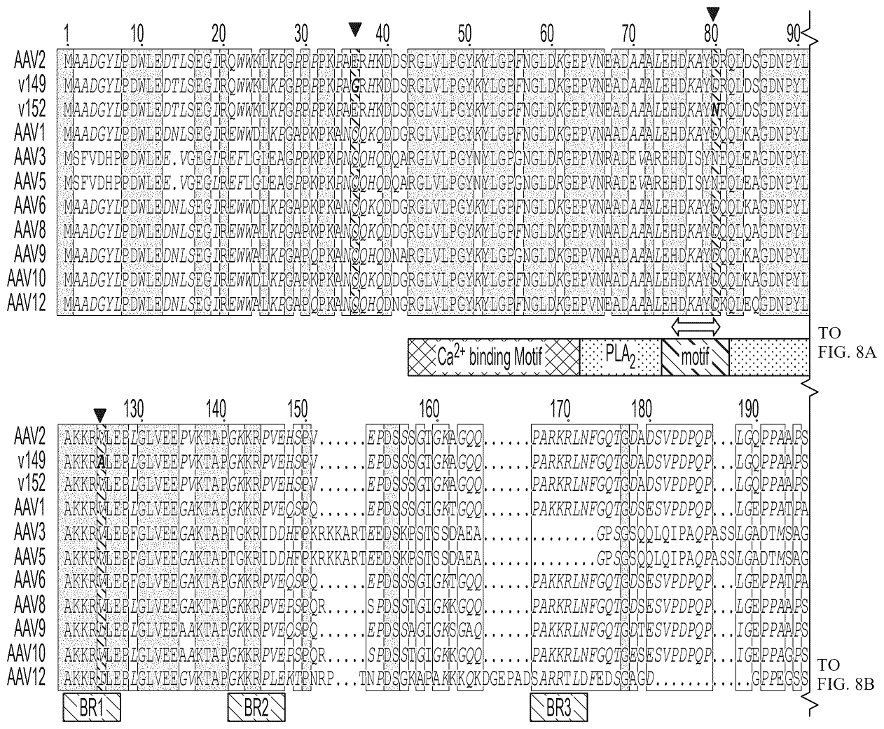

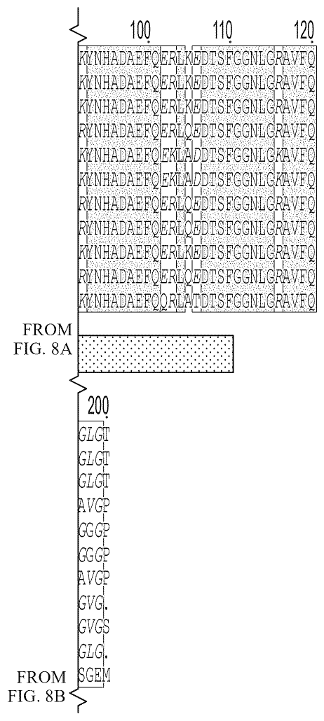

The adeno-associated virus (AAV) viral capsid is composed of three structural proteins (VPs), designated VP1, VP2, and VP3, which are encoded by the cap gene open reading frame. The VPs share common central and C-terminal regions, with N-terminal extensions of 65 amino acids for VP2 and VP3, and an additional 137 N-terminal amino acid region which forms the unique region of VP1 (i.e., VP1 unique, or VP1u). In some embodiments, the VP1u region encompasses amino acid residues 1-200 of AAV2. The VP1u region is an unstructured region of the capsid that is internal within the protein, and is exposed upon viral entry into the cell, where it is then trafficked via retrograde transport through endosomes and lysosomes. VP1u plays crucial roles in endosomal trafficking and escape, nuclear entry, and genome release. The VP1u region is highly conserved across AAV serotypes, for example as described by Popa-Wagner et al., J Gen Virol. 2012; 93(Pt 9):1887-98; and Viney et al. J Virol. 2021; 95(7). FIGS. 8A-8B show a protein sequence alignment indicating the high level of conservation of VP1u across different AAV serotypes.

The disclosure herein provides adeno-associated virus (AAV) capsid proteins having at least three amino acid mutations within the VP1 unique (VP1u) region at amino acid positions corresponding to positions E36, D80, and V125 with reference to amino acid position numbering of a wild-type AAV2 capsid protein (e.g., SEQ ID NO: 1). In some aspects, the disclosure provides a method for delivering a transgene to a target cell (e.g., ocular cell) in a subject, the method comprising administering (e.g., intravitreally) to the subject an rAAV comprising an isolated nucleic acid encoding one or more gene products, and an AAV capsid protein having at least three amino acid mutations within the VP1u region at amino acid positions corresponding to positions E36, D80, and V125 with reference to amino acid position numbering of a wild-type AAV2 capsid protein (e.g., SEQ ID NO: 1). In some embodiments, the AAV capsid protein is of a serotype selected from AAV1, AAV2, AAV3, AAV4, AAV5, AAV6, AAV7, AAV8, AAV9, AAV10, AAV9.PHPeB, MYOAAV, AAVrh74, AAV2.5T, AAV2/8, AAV2/6, AAVrh32.33, Anc80, NP40, NP59, and LKO3.

In some embodiments, an AAV capsid protein comprises an amino acid mutation at an amino acid position corresponding to position E36 with reference to amino acid position numbering of a wild-type AAV2 capsid protein. In some embodiments, an amino acid mutation at a position corresponding to position E36 is an amino acid substitution. An amino acid substitution at position E36 may result in loss of the negative charge of the side chain at position 36. In some embodiments, an amino acid substitution at position E36 introduces an uncharged, nonpolar amino acid at position E36 (e.g., E36A, E36G, E36I, E36L, E36P, E36V, E36F, E36W, E36C, or E36M). In some embodiments, an amino acid substitution at position E36 is E36M. In some embodiments, an AAV capsid protein comprises (i) an amino acid mutation at an amino acid position corresponding to position E36, and (ii) at least 80%, 90%, 95%, 97%, or 98% sequence homology (or sequence identity) to a wild-type AAV2 capsid protein (SEQ ID NO: 1).

In some embodiments, an AAV capsid protein comprises an amino acid mutation at an amino acid position corresponding to position D80 with reference to amino acid position numbering of a wild-type AAV2 capsid protein (e.g., SEQ ID NO: 1). In some embodiments, an amino acid mutation at a position corresponding to position D80 is an amino acid substitution. An amino acid substitution at position D80 may result in loss of the negative charge of the side chain at position D80. In some embodiments, an amino acid substitution at position D80 introduces an uncharged, polar amino acid at position D80 (e.g., D80N, D80Q, D80S, D80T, or D80Y). In some embodiments, an amino acid substitution at position D80 is D80N. In some embodiments, an AAV capsid protein comprises (i) an amino acid mutation at an amino acid position corresponding to position D80, and (ii) at least 80%, 90%, 95%, 97%, or 98% sequence homology (or sequence identity) to a wild-type AAV2 capsid protein (SEQ ID NO: 1).

In some embodiments, an AAV capsid protein comprises an amino acid mutation at an amino acid position corresponding to position V125 with reference to amino acid position numbering of a wild-type AAV2 capsid protein (e.g., SEQ ID NO: 1). In some embodiments, an amino acid mutation at a position corresponding to position V125 is an amino acid substitution. An amino acid substitution at position V125 may be a conservative substitution. In some embodiments, an amino acid substitution at position V125 is V125A, V125G, V1251, V125L, V125P, V125V, V125F, V125W, V125C, or V125M. In some embodiments, an AAV capsid protein comprises (i) an amino acid mutation at an amino acid position corresponding to position V125, and (ii) at least 80%, 90%, 95%, 97%, or 98% sequence homology (or sequence identity) to a wild-type AAV2 capsid protein (SEQ ID NO: 1).

In some embodiments, an AAV capsid protein comprises amino acid mutations at amino acid positions corresponding to positions E36, D80, and V125 (e.g., E36G, D80N, and V125A) with reference to amino acid position numbering of a wild-type AAV2 capsid protein (e.g., SEQ ID NO: 1). In some embodiments, an AAV capsid protein comprises or consists of the amino acid sequence set forth in SEQ ID NO: 4. In some embodiments, a VP1u region of an AAV capsid protein comprises or consists of an amino acid sequence that is at least 70% identical to the amino acid sequence set forth in SEQ ID NO: 1.

Mutations contemplated herein, with respect to an amino acid sequence, include, without limitation, substitutions, additions, and deletions. An amino acid “substitution” is a change in a single amino acid relative to a reference amino acid sequence. For example, an amino acid substitution at an amino acid position corresponding to position E36 with reference to amino acid position numbering of a wild-type AAV2 capsid protein (e.g., SEQ ID NO: 1) involves a change from glutamic acid (Glu/E) to another amino acid at that position (e.g., Gly/G).

An amino acid substitution may result in a change in charge of the side chain of the amino acid position (e.g., from negatively charged to positively charged). In some embodiments, an amino acid substitution results in a change in polarity or hydrophobicity of the side chain of the amino acid position. In some embodiments, an amino acid substitution is a conservative substitution (e.g., a change from valine to alanine). In some embodiments, a “conservative” amino acid substitution results in a different amino acid at that position that has an “equivalent” charge, polarity, and/or chemical class (defined by the amino acid side chain). Table 1 provides the 20 naturally occurring amino acids with a description of corresponding charge, polarity, and chemical class. For example, arginine has an equivalent charge to histidine and lysine; an equivalent polarity to asparagine, glutamine, serine, threonine, tyrosine aspartic acid, glutamic acid, arginine, histidine, and lysine; and an equivalent chemical class/side chain to histidine and lysine.

TABLE 1
Amino Acids

| | | | |
|---|---|---|---|
| Amino acid | Abbreviation | Charge | Polarity |

| | | | | |
|---|---|---|---|---|
| Alanine | Ala | A | uncharged | nonpolar |
| Glycine | Gly | G | uncharged | nonpolar |
| Isoleucine | Ile | I | uncharged | nonpolar |
| Leucine | Leu | L | uncharged | nonpolar |
| Proline | Pro | P | uncharged | nonpolar |
| Valine | Val | V | uncharged | nonpolar |
| Phenylalanine | Phe | F | uncharged | nonpolar |
| Tryptophan | Trp | W | uncharged | nonpolar |
| Cysteine | Cys | C | uncharged | nonpolar |
| Methionine | Met | M | uncharged | nonpolar |
| Asparagine | Asn | N | uncharged | polar |
| Glutamine | Gln | Q | uncharged | polar |
| Serine | Ser | S | uncharged | polar |
| Threonine | Thr | T | uncharged | polar |
| Tyrosine | Tyr | Y | uncharged | polar |
| Aspartic acid | Asp | D | negative | polar |
| Glutamic acid | Glu | E | negative | polar |
| Arginine | Arg | R | positive | polar |
| Histidine | His | H | positive | polar |
| Lysine | Lys | K | positive | polar |

“Homology” refers to the percent identity between two polynucleotide or two polypeptide moieties. The term “substantial homology”, when referring to a nucleic acid, or fragment thereof, indicates that, when optimally aligned with appropriate nucleotide insertions or deletions with another nucleic acid (or its complementary strand), there is nucleotide sequence identity in about 90 to 100% of the aligned sequences. When referring to a polypeptide, or fragment thereof, the term “substantial homology” indicates that, when optimally aligned with appropriate gaps, insertions or deletions with another polypeptide, there is nucleotide sequence identity in about 90 to 100% of the aligned sequences. The term “highly conserved” means at least 80% identity, preferably at least 90% identity, and more preferably, over 97% identity. In some cases, highly conserved may refer to 100% identity. Identity is readily determined by one of skill in the art by, for example, the use of algorithms and computer programs known by those of skill in the art.

As described herein, alignments between sequences of nucleic acids or polypeptides are performed using any of a variety of publicly or commercially available Multiple Sequence Alignment Programs, such as “Clustal W”, accessible through Web Servers on the internet. Alternatively, Vector NTI utilities may also be used. There are also a number of algorithms known in the art that can be used to measure nucleotide sequence identity, including those contained in the programs described above. As another example, polynucleotide sequences can be compared using BLASTN, which provides alignments and percent sequence identity of the regions of the best overlap between the query and search sequences. Similar programs are available for the comparison of amino acid sequences, e.g., the “Clustal X” program, BLASTP. Typically, any of these programs are used at default settings, although one of skill in the art can alter these settings as needed. Alternatively, one of skill in the art can utilize another algorithm or computer program that provides at least the level of identity or alignment as that provided by the referenced algorithms and programs. Alignments may be used to identify corresponding amino acids between two proteins or peptides. A “corresponding amino acid” is an amino acid of a protein or peptide sequence that has been aligned with an amino acid of another protein or peptide sequence. Corresponding amino acids may be identical or non-identical. A corresponding amino acid that is a non-identical amino acid may be referred to as a variant amino acid.

In some aspects, the disclosure relates to an AAV2 MCi capsid protein (e.g., an isolated nucleic acid encoding an AAV2 MCi capsid protein, a rAAV comprising an AAV2 MCi capsid protein, etc.), or a capsid protein having substantial homology to a AAV2 MCi capsid protein. In some embodiments, a capsid protein having substantial homology to an AAV2 MCi capsid protein is at least 50%, 60%, 70%, 80%, 90%, 95%, or 99% identical to the amino acid sequence set forth in SEQ ID NO: 4. In some embodiments, a capsid protein having substantial homology to an AAV2 MCi capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 4.

In some aspects, the disclosure relates to an AAV8 MCi capsid protein (e.g., an isolated nucleic acid encoding an AAV8 MCi capsid protein, a rAAV comprising an AAV8 MCi capsid protein, etc.), or a capsid protein having substantial homology to a AAV8 MCi capsid protein. In some embodiments, an AAV8 MCi capsid protein comprises at least a segment (or the entirety) of an AAV2 VP1u region that is linked (or grafted) onto an AAV8 capsid protein (e.g., a wild-type AAV8 capsid protein) such that the segment (or the entirety) of the AAV2 VP1u region replaces the VP1u region of the AAV8 capsid protein. In some embodiments, an AAV8 MCi capsid protein comprises a glycine (G) at position 36, an asparagine (N) at position 80, and an alanine (A) at position 125 with reference to amino acid position numbering of a wild-type AAV2 capsid protein. In some embodiments, a capsid protein having substantial homology to an AAV8 MCi capsid protein is at least 50%, 60%, 70%, 80%, 90%, 95%, or 99% identical to the amino acid sequence set forth in SEQ ID NO: 14. In some embodiments, a capsid protein having substantial homology to an AAV8 MCi capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 8.

In some aspects, the disclosure relates to an AAV9 MCi capsid protein (e.g., an isolated nucleic acid encoding an AAV9 MCi capsid protein, a rAAV comprising an AAV9 MCi capsid protein, etc.), or a capsid protein having substantial homology to a AAV9 MCi capsid protein. In some embodiments, an AAV9 MCi capsid protein comprises at least a segment (or the entirety) of an AAV2 VP1u region that is linked (or grafted) onto an AAV9 capsid protein (e.g., a wild-type AAV9 capsid protein) such that the segment (or the entirety) of the AAV2 VP1u region replaces the VP1u region of the AAV9 capsid protein. In some embodiments, an AAV9 MCi capsid protein comprises a glycine (G) at position 36, an asparagine (N) at position 80, and an alanine (A) at position 125 with reference to amino acid position numbering of a wild-type AAV2 capsid protein. In some embodiments, a capsid protein having substantial homology to an AAV9 MCi capsid protein is at least 50%, 60%, 70%, 80%, 90%, 95%, or 99% identical to the amino acid sequence set forth in SEQ ID NO: 15. In some embodiments, a capsid protein having substantial homology to an AAV9 MCi capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 15.

In some aspects, the disclosure relates to an AAV1 MCi capsid protein (e.g., an isolated nucleic acid encoding an AAV1 MCi capsid protein, a rAAV comprising an AAV1 MCi capsid protein, etc.), or a capsid protein having substantial homology to a AAV1 MCi capsid protein. In some embodiments, an AAV1 MCi capsid protein comprises at least a segment (or the entirety) of an AAV2 VP1u region that is linked (or grafted) onto an AAV1 capsid protein (e.g., a wild-type AAV1 capsid protein) such that the segment (or the entirety) of the AAV2 VP1u region replaces the VP1u region of the AAV1 capsid protein. In some embodiments, an AAV1 MCi capsid protein comprises a glycine (G) at position 36, an asparagine (N) at position 80, and an alanine (A) at position 125 with reference to amino acid position numbering of a wild-type AAV2 capsid protein. In some embodiments, a capsid protein having substantial homology to an AAV1 MCi capsid protein is at least 50%, 60%, 70%, 80%, 90%, 95%, or 99% identical to the amino acid sequence set forth in SEQ ID NO: 16. In some embodiments, a capsid protein having substantial homology to an AAV1 MCi capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 16.

In some aspects, the disclosure relates to an AAV3B MCi capsid protein (e.g., an isolated nucleic acid encoding an AAV3B MCi capsid protein, a rAAV comprising an AAV3B MCi capsid protein, etc.), or a capsid protein having substantial homology to a AAV3B MCi capsid protein. In some embodiments, an AAV3B MCi capsid protein comprises at least a segment (or the entirety) of an AAV2 VP1u region that is linked (or grafted) onto an AAV3B capsid protein (e.g., a wild-type AAV3B capsid protein) such that the segment (or the entirety) of the AAV2 VP1u region replaces the VP1u region of the AAV3B capsid protein. In some embodiments, an AAV3B MCi capsid protein comprises a glycine (G) at position 36, an asparagine (N) at position 80, and an alanine (A) at position 125 with reference to amino acid position numbering of a wild-type AAV2 capsid protein. In some embodiments, a capsid protein having substantial homology to an AAV3B MCi capsid protein is at least 50%, 60%, 70%, 80%, 90%, 95%, or 99% identical to the amino acid sequence set forth in SEQ ID NO: 17. In some embodiments, a capsid protein having substantial homology to an AAV3B MCi capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 17.

In some aspects, the disclosure relates to an AAV5 MCi capsid protein (e.g., an isolated nucleic acid encoding an AAV5 MCi capsid protein, a rAAV comprising an AAV5 MCi capsid protein, etc.), or a capsid protein having substantial homology to a AAV5 MCi capsid protein. In some embodiments, an AAV5 MCi capsid protein comprises at least a segment (or the entirety) of an AAV2 VP1u region that is linked (or grafted) onto an AAV5 capsid protein (e.g., a wild-type AAV5 capsid protein) such that the segment (or the entirety) of the AAV2 VP1u region replaces the VP1u region of the AAV5 capsid protein. In some embodiments, an AAV5 MCi capsid protein comprises a glycine (G) at position 36, an asparagine (N) at position 80, and an alanine (A) at position 125 with reference to amino acid position numbering of a wild-type AAV2 capsid protein. In some embodiments, a capsid protein having substantial homology to an AAV5 MCi capsid protein is at least 50%, 60%, 70%, 80%, 90%, 95%, or 99% identical to the amino acid sequence set forth in SEQ ID NO: 18. In some embodiments, a capsid protein having substantial homology to an AAV5 MCi capsid protein comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 18.

In some aspects, the disclosure relates to a VP1u MCi region (e.g., an isolated nucleic acid encoding VP1u MCi, a rAAV having a capsid protein comprising VP1u MCi, etc.), or a capsid protein having substantial homology to a VP1u MCi region. In some embodiments, a capsid protein having substantial homology to a VP1u MCi region is at least 50%, 60%, 70%, 80%, 90%, 95%, or 99% identical to the amino acid sequence set forth in SEQ ID NO: 1. In some embodiments, a capsid protein having substantial homology to a VP1u MCi region comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 amino acid substitutions, insertions, or deletions, relative to the amino acid sequence set forth in SEQ ID NO: 1.

The disclosure relates, in some aspects, to the discovery that vectors packaged with AAV MCi (e.g., AAV2 MCi) capsids have better transduction in mammalian cells (e.g., HeLa cells) relative to rAAVs having certain other AAV capsid proteins (e.g., AAV2 capsid proteins or AAV2 7m8 capsid proteins). In some embodiments, rAAVs having AAV MCi (e.g., AAV2 MCi) capsid proteins transduce mammalian cells (e.g., HeLa cells) about 1.5 to 3.5 times more efficiently than rAAVs having certain other AAV capsid proteins (e.g., AAV2 capsid proteins or AAV2 7m8 capsid proteins). In some embodiments, rAAVs having AAV MCi (e.g., AAV2 MCi) capsid proteins transduce mammalian cells (e.g., HeLa cells) about 50% to about 250% (e.g., 50%, 60%, 70%, 80%, 90%, 100%, 110%, 120%, 130%, 140%, 150%, 160%, 170%, 180%, 190%, 200%, 210%, 220%, 230%, 240%, 250%, etc.) more efficiently than rAAVs having any certain other AAV capsid protein (e.g., AAV2 capsid proteins or AAV2 7m8 capsid proteins).

Aspects of the disclosure relate to the unexpectedly improved ocular cell transduction efficiency of AAV MCi (e.g., AAV2 MCi) capsid proteins (e.g., rAAVs comprising AAV2 MCi capsid proteins) relative to rAAVs having AAV2 or AAV2 7m8 capsid proteins. In some embodiments, AAV MCi-containing rAAVs transduce ocular cells at least 5%, 10%, 15%, 20%, 25%, 30%, 40%, 50%, 100%, 200%, 500%, 1000%, or more efficiently than AAV2- or AAV2 7m8-containing rAAVs. There are several ways to test the efficiency of rAAV transduction. For example, one can measure the abundance of viral genomes, transgenes, or transgene gene product(s) in a transduced cell or tissue at a particular appropriate time point or over a period of time. Abundance be measured through any technique known in the art, including, but not limited to, PCR (e.g., RT-PCR, ddPCR, etc.), enzyme-linked assays (e.g., ELISA), Western blotting, and microscopy techniques (e.g., immunohistochemistry, immunofluorescence, etc.). In some embodiments, high abundance, especially at earlier timepoints, indicates enhanced transduction efficiency of a particular rAAV compared to an rAAV with certain other AAV capsid proteins.

Aspects of the disclosure relate to the unexpectedly improved mammalian cell (e.g., HeLa cell) internalization of vectors packaged in rAAVs containing AAV MCi (e.g., AAV2 MCi) capsid proteins relative to rAAVs having AAV2 or AAV2 7m8 capsid proteins. In some embodiments, AAV MCi-containing rAAVs confer at least 5%, 10%, 15%, 20%, 25%, 30%, 40%, 50%, 100%, 200%, 500%, 1000%, or more higher internalization into mammalian cells (e.g., HeLa cells) than AAV2- or AAV2 7m8-containing rAAVs.

“Internalization”, as used herein, refers to the binding of rAAV capsid proteins to extracellular receptors of a target cell, leading to the uptake of rAAV with said capsid proteins, and the trafficking of its contents to the nucleus of a host cell where the contents (e.g., nucleic acids) may then be expressed. In some embodiments, increased internalization of vectors is due to enhanced binding between capsid proteins and host cell receptors. In some embodiments, increased internalization of vectors is due to enhanced trafficking of the vectors between the host cell membrane and nucleus. Internalization can be measured through any technique known in the art, including, but not limited to, PCR (e.g., RT-PCR, ddPCR, etc.), enzyme-linked assays (e.g., ELISA), Western blotting, and microscopy techniques (e.g., immunohistochemistry, immunofluorescence, etc.

Aspects of the disclosure relate to certain AAV capsid proteins that are serologically distinct from other AAV capsid proteins (e.g., AAV1, AAV2, AAV3B, AAV8, AAV9, AAVrh.8, AAVrh.10, etc.). Without wishing to be bound by any particular theory, rAAVs comprising AAV MCi (e.g., AAV2 MCi) capsid proteins are not subject to the neutralizing antibody response in a subject that is seropositive for antibodies against certain other AAV capsids. Accordingly, in some embodiments, rAAVs comprising capsid proteins as described herein may be useful as a second-line therapy for delivery of transgenes to subjects that have previously been administered AAV therapies, or that are seropositive for certain AAV capsid neutralizing antibodies.

### Isolated Nucleic Acids

In some aspects, the disclosure relates to isolated nucleic acids encoding certain AAV capsid protein variants or capsid peptide variants (e.g., AAV2 MC1capsid protein or VP1u MCi). A “nucleic acid” sequence refers to a DNA or RNA sequence. In some embodiments, the term nucleic acid captures sequences that include any of the known base analogues of DNA and RNA such as, but not limited to 4-acetylcytosine, 8-hydroxy-N6-methyladenosine, aziridinylcytosine, pseudoisocytosine, 5-(carboxyhydroxyl-methyl) uracil, 5-fluorouracil, 5-bromouracil, 5-carboxymethylaminomethyl-2-thiouracil, 5-carboxymethyl-aminomethyluracil, dihydrouracil, inosine, N6-isopentenyladenine, 1-methyladenine, 1-methylpseudo-uracil, 1-methylguanine, 1-methylinosine, 2,2-dimethyl-guanine, 2-methyladenine, 2-methylguanine, 3-methyl-cytosine, 5-methylcytosine, N6-methyladenine, 7-methylguanine, 5-methylaminomethyluracil, 5-methoxy-amino-methyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, -uracil-5-oxyacetic acid methylester, uracil-5-oxyacetic acid, pseudouracil, queosine, 2-thiocytosine, and 2,6-diaminopurine.

In some embodiments, proteins and nucleic acids of the disclosure are isolated. As used herein, the term “isolated” means artificially obtained or produced. As used herein with respect to nucleic acids, the term “isolated” generally means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one that is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. As used herein with respect to proteins or peptides, the term “isolated” generally refers to a protein or peptide that has been artificially obtained or produced (e.g., by chemical synthesis, by recombinant DNA technology, etc.).

It should be appreciated that conservative amino acid substitutions may be made to provide functionally equivalent variants, or homologs of the capsid proteins. In some aspects the disclosure embraces sequence alterations that result in conservative amino acid substitutions. As used herein, a conservative amino acid substitution refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g., Molecular Cloning: A Laboratory Manual, J. Sambrook, et al., eds., Second Edition, Cold Spring Harbor Laboratory Press, Cold Spring Harbor, New York, 1989, or Current Protocols in Molecular Biology, F.M. Ausubel, et al., eds., John Wiley & Sons, Inc., New York. Conservative substitutions of amino acids include substitutions made among amino acids within the following groups: (a) M, I, L, V; (b) F, Y, W; (c) K, R, H; (d) A, G; (e) S, T; (f) Q, N; and (g) E, D. Therefore, one can make conservative amino acid substitutions to the amino acid sequence of the proteins and polypeptides disclosed herein.

Recombinant AAVs (rAAVs)

In some aspects, the disclosure provides isolated AAVs. As used herein with respect to AAVs, the term “isolated” refers to an AAV that has been artificially obtained or produced. Isolated AAVs may be produced using recombinant methods. Such AAVs are referred to herein as “recombinant AAVs”. Recombinant AAVs (rAAVs) preferably have tissue-specific targeting capabilities, such that a transgene of the rAAV will be delivered specifically to one or more predetermined tissue(s) (e.g., ocular cells). The AAV capsid is an important element in determining these tissue-specific targeting capabilities. Thus, an rAAV having a capsid appropriate for the tissue being targeted can be selected. In some embodiments, the rAAV comprises a capsid protein having an amino acid sequence as set forth in SEQ ID NO: 4, or a protein having substantial homology thereto. In some embodiments, the rAAV comprises a capsid protein of any serotype wherein the VP1u region of the protein comprises or consists of an amino acid sequence that is at least 70% identical to the amino acid sequence set forth in SEQ ID NO: 1.

Methods for obtaining rAAVs having a desired capsid protein are well known in the art. See, for example, US 2003/0138772, the contents of which are incorporated herein by reference in their entirety. Typically the methods involve culturing a host cell which contains a nucleic acid sequence encoding an AAV capsid protein (e.g., a nucleic acid encoding a polypeptide having a sequence set forth as SEQ ID NOs: 7 or 9) or a fragment thereof; a functional rep gene; a rAAV vector composed of AAV inverted terminal repeats (ITRs) and a transgene; and sufficient helper functions to permit packaging of the rAAV vector into the AAV capsid proteins. In some embodiments, capsid proteins are structural proteins encoded by a cap gene of an AAV. In some embodiments, AAVs comprise three capsid proteins, virion proteins 1 to 3 (named VP1, VP2, and VP3), all of which may be expressed from a single cap gene. Accordingly, in some embodiments, the VP1, VP2, and VP3 proteins share a common core sequence. In some embodiments, the molecular weights of VP1, VP2, and VP3 are respectively about 85 kDa, about 73 kDa, and about 62 kDa. In some embodiments, upon translation, capsid proteins form a spherical 60-mer protein shell around the viral genome. In some embodiments, the protein shell is primarily comprised of a VP3 capsid protein (e.g., 1 VP1: 1 VP2: 10 VP3). In some embodiments, the functions of the capsid proteins are to protect the viral genome, deliver the genome and interact with the host. In some aspects, capsid proteins deliver the viral genome to a host in a tissue-specific manner. In some embodiments, VP1 and/or VP2 capsid proteins may contribute to the tissue tropism of the packaged AAV. In some embodiments, when present in the capsid proteins of an rAAV, the VP1u MCi region disclosed herein enhances interaction with (e.g., cell surface binding) and/or internalization of a packaged transgene into mammalian cells (e.g., HeLa cells or ocular cells). In some embodiments, the tissue tropism of the packaged AAV is determined by the VP3 capsid protein. In some embodiments, the tissue tropism of the packaged AAV is determined by the VP1 capsid protein. In some embodiments, the tissue tropism of an AAV is enhanced or changed by mutations occurring in the capsid proteins. In some embodiments, the tropism of an AAV is enhanced or changed by mutations in the VP1u region of the capsid proteins.

In some embodiments, the AAV variants described herein are variants of AAV2. AAV2 is known to efficiently transduce human ocular tissue (eyes) and ocular cells. Accordingly, in some embodiments, the AAV2 variants described herein may be useful for delivering gene therapy to ocular tissue (eyes) and ocular cells. In some embodiments, AAV capsid proteins described herein are useful for targeting other tissues, for example muscle tissue, liver tissue, or cardiac tissue.

In some embodiments, the AAV variants described herein comprise VP1 proteins of any serotype (e.g., AAV1, AAV2, AAV3B, AAV8, AAV9, AAVrh.8, AAVrh.10, etc.) wherein the VP1u region of the capsid protein comprises or consists of a sequence that is at least 70% identical to the sequence set forth in SEQ ID NO: 1. Thus, VP1u MCi may replace the wild-type VP1u region of any capsid protein to increase transgene internalization and/or capsid binding to mammalian cells.

In some embodiments, AAV variants described herein may be useful for delivering gene therapy to ocular tissue (e.g., tissue or cells of the eye). Accordingly, in some embodiments, AAV variants described herein may be useful for the treatment of ocular disorders. As used herein, an “ocular disorder” is a disease or condition of the eye. An ocular disease may affect the eye, sclera, cornea, anterior chamber, posterior chamber, iris, pupil, lens, vitreous humor, retina, or optic nerve. An ocular disorder may be of a genetic origin, either inherited or acquired through a somatic mutation. Non-limiting examples of ocular diseases and disorders include but are not limited to: age-related macular degeneration, retinopathy, diabetic retinopathy, macular edema, glaucoma, retinitis pigmentosa and eye cancer.

The components to be cultured in the host cell to package a rAAV vector in an AAV capsid may be provided to the host cell in trans. Alternatively, any one or more of the required components (e.g., recombinant AAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

The recombinant AAV vector, rep sequences, cap sequences, and helper functions required for producing the rAAV of the disclosure may be delivered to the packaging host cell using any appropriate genetic element (vector). In some embodiments, a single nucleic acid encoding all three capsid proteins (e.g., VP1, VP2 and VP3) is delivered into the packaging host cell in a single vector. In some embodiments, nucleic acids encoding the capsid proteins are delivered into the packaging host cell by two vectors; a first vector comprising a first nucleic acid encoding two capsid proteins (e.g., VP1 and VP2) and a second vector comprising a second nucleic acid encoding a single capsid protein (e.g., VP3). In some embodiments, three vectors, each comprising a nucleic acid encoding a different capsid protein, are delivered to the packaging host cell. The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y. Similarly, methods of generating rAAV virions are well known and the selection of a suitable method is not a limitation on the present disclosure. See, e.g., K. Fisher et al, J. Virol., 70:520-532 (1993) and U.S. Pat. No. 5,478,745.

In some embodiments, recombinant AAVs may be produced using the triple transfection method (described in detail in U.S. Pat. No. 6,001,650). Typically, the recombinant AAVs are produced by transfecting a host cell with a recombinant AAV vector (comprising a transgene) to be packaged into AAV particles, an AAV helper function vector, and an accessory function vector. An AAV helper function vector encodes the “AAV helper function” sequences (e.g., rep and cap), which function in trans for productive AAV replication and encapsidation. Preferably, the AAV helper function vector supports efficient AAV vector production without generating any detectable wild-type AAV virions (e.g., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (e.g., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced inside the cell (e.g., across the cell membrane). A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of recombinant AAVs. The term includes the progeny of the original cell that has been transfected. Thus, a “host cell” as used herein may refer to a cell that has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the terms “recombinant cell” refers to a cell into which an exogenous DNA segment, such as DNA segment that leads to the transcription of a biologically-active polypeptide or production of a biologically active nucleic acid such as an RNA, has been introduced.

Cells may also be transfected with a vector (e.g., helper vector) that provides helper functions to the AAV. The vector providing helper functions may provide adenovirus functions, including, e.g., E1a, E1b, E2a, and E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

As used herein, the term “vector” includes any genetic element, such as a plasmid, phage, transposon, cosmid, chromosome, artificial chromosome, virus, virion, etc., that is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment (e.g., nucleic acid sequence) to be transcribed is positioned under the transcriptional control of a promoter. A “promoter” refers to a DNA sequence recognized by the synthetic machinery of the cell, or introduced synthetic machinery, that is required to initiate the specific transcription of a gene. The phrases “operatively positioned,” “under control” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

In some cases, an isolated capsid gene can be used to construct and package recombinant AAVs, using methods well known in the art, to determine functional characteristics associated with the capsid protein encoded by the gene. For example, isolated capsid genes can be used to construct and package a recombinant AAV (rAAV) comprising a reporter gene (e.g., B-Galactosidase, GFP, Luciferase, etc.). The rAAV can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The foregoing methods for packaging recombinant vectors in desired AAV capsids to produce the rAAVs of the disclosure are not meant to be limiting and other suitable methods will be apparent to the skilled artisan.

rAAV Vectors

“Recombinant AAV (rAAV) vectors” of the disclosure are typically composed of, at a minimum, a transgene (e.g., a transgene encoding one or more gene products) and its regulatory sequences, and 5′ and 3′ AAV inverted terminal repeats (ITRs). It is this recombinant AAV vector which is packaged into a capsid protein and delivered to a selected target cell. In some embodiments, the transgene is a nucleic acid sequence, heterologous to the vector sequences, that encodes a polypeptide, protein, functional RNA molecule (e.g., miRNA, miRNA inhibitor) or other gene product, of interest. The nucleic acid coding sequence is operatively linked to regulatory components in a manner that permits transgene transcription, translation, and/or expression in a cell of a target tissue.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al, “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In some embodiments, the disclosure provides a self-complementary AAV vector. As used herein, the term “self-complementary AAV vector” (scAAV) refers to a vector containing a double-stranded vector genome generated by the absence of a terminal resolution site (TR) from one of the ITRs of the AAV. The absence of a TR prevents the initiation of replication at the vector terminus where the TR is not present. In general, scAAV vectors generate single-stranded, inverted repeat genomes, with a wild-type (wt) AAV TR at each end and a mutated TR (mTR) in the middle.

In some embodiments, the rAAVs of the present disclosure are pseudotyped rAAVs. Pseudotyping is the process of producing viruses or viral vectors in combination with foreign viral envelope proteins. The result is a pseudotyped virus particle. With this method, the foreign viral envelope proteins can be used to alter host tropism or an increased/decreased stability of the virus particles. In some aspects, a pseudotyped rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudotyped rAAV refers to an AAV comprising an inverted terminal repeat (ITR) of one AAV serotype and a capsid protein of a different AAV serotype. For example, a pseudotyped AAV vector containing the ITRs of serotype X encapsidated with the proteins of Y will be designated as AAVX/Y (e.g., AAV2/1 has the ITRs of AAV2 and the capsid of AAV1). In some embodiments, pseudotyped rAAVs may be useful for combining the tissue-specific targeting capabilities of a capsid protein from one AAV serotype with the viral DNA from another AAV serotype, thereby allowing targeted delivery of a transgene to a target tissue.

In some embodiments, the rAAV described herein is a single stranded AAV (ssAAV). An ssAAV, as used herein, refers to an rAAV with the coding sequence and complementary sequence of the transgene expression cassette on separate strands and are packaged in separate viral capsids.

In addition to the major elements identified above for the recombinant AAV vector, the vector also includes conventional control elements necessary which are operably linked to the transgene in a manner which permits its transcription, translation and/or expression in a cell transfected with the plasmid vector or infected with the virus produced by the disclosure. As used herein, “operably linked” sequences include both expression control sequences that are contiguous with the gene of interest and expression control sequences that act in trans or at a distance to control the gene of interest.

Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (e.g., Kozak consensus sequence); sequences that enhance protein stability; and when desired, sequences that enhance secretion of the encoded product. A great number of expression control sequences, including promoters that are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly, two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the transgene sequences and before the 3′ AAV ITR sequence. An rAAV construct useful in the present disclosure may also contain an intron, desirably located between the promoter/enhancer sequence and the transgene. One possible intron sequence is derived from SV-40, and is referred to as the SV-40 T intron sequence. Another vector element that may be used is an internal ribosome entry site (IRES). An IRES sequence is used to produce more than one polypeptide from a single gene transcript. An IRES sequence would be used to produce a protein that contains more than one polypeptide chain. Selection of these and other common vector elements are conventional, and many such sequences are available (See, e.g., Sambrook et al, and references cited therein at, for example, pages 3.18 3.26 and 16.17 16.27 and Ausubel et al., Current Protocols in Molecular Biology, John Wiley & Sons, New York, 1989). In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459). The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931.; and Klump, H et al., Gene Therapy, 2001; 8: 811-817).

The precise nature of the regulatory sequences needed for gene expression in host cells may vary between species, tissues, or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences. The choice and design of an appropriate vector is within the ability and discretion of one of ordinary skill in the art.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al, Cell, 41:521-530 (1985)], the SV40 promoter, the dihydrofolate reductase promoter, the 3-actin promoter, the phosphoglycerol kinase (PGK) promoter, and the EF1α promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al, Proc. Natl. Acad. Sci. USA, 93:3346-3351 (1996)), the tetracycline-repressible system (Gossen et al, Proc. Natl. Acad. Sci. USA, 89:5547-5551 (1992)), the tetracycline-inducible system (Gossen et al, Science, 268:1766-1769 (1995), see also Harvey et al, Curr. Opin. Chem. Biol., 2:512-518 (1998)), the RU486-inducible system (Wang et al, Nat. Biotech., 15:239-243 (1997) and Wang et al, Gene Ther., 4:432-441 (1997)) and the rapamycin-inducible system (Magari et al, J. Clin. Invest., 100:2865-2872 (1997)). Still other types of inducible promoters that may be useful in this context are those that are regulated by a specific physiological state, e.g., temperature, acute phase, a particular differentiation state of the cell, or in replicating cells only.

In another embodiment, the native promoter for the transgene will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, a nucleic acid described herein utilizes a tissue-specific promoter (e.g., to promote expression of a transgene in a tissue-specific manner). In some embodiments, a tissue-specific promoter is an eye-specific promoter (e.g., an ocular gene promoter). Examples of eye-specific promoters include retinoschisin proximal promoter, interphotoreceptor retinoid-binding protein enhancer (RS/IRBPa), rhodopsin kinase (RK), RPE65, and human cone opsin promoter.

In some embodiments, the regulatory sequences impart tissue-specific gene expression capabilities. In some cases, the tissue-specific regulatory sequences bind tissue-specific transcription factors that induce transcription in a tissue specific manner. Such tissue-specific regulatory sequences (e.g., promoters, enhancers, etc.) are well known in the art. Exemplary tissue-specific regulatory sequences include, but are not limited to the following tissue specific promoters: a liver-specific thyroxin binding globulin (TBG) promoter, an insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, a gastrointestinal-specific mucin-2 promoter, an eye-specific retinoschisin promoter, an eye-specific K12 promoter, a respiratory tissue-specific CC10 promoter, a respiratory tissue-specific surfactant protein C (SP-C) promoter, a breast tissue-specific PRC1 promoter, a breast tissue-specific RRM2 promoter, a urinary tract tissue-specific uroplakin 2 (UPII) promoter, a uterine tissue-specific lactoferrin promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor α-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), among others which will be apparent to the skilled artisan.

In some embodiments, one or more bindings sites for one or more of miRNAs are incorporated in a transgene of a rAAV vector, to inhibit the expression of the transgene in one or more tissues of a subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RNA-induced silencing complexes (RISCs) and provide highly efficient inhibition of expression. The target site sequence may comprise a total of 5-100, 10-60, or more nucleotides. The target site sequence may comprise at least 5 nucleotides of the sequence of a target gene binding site.

The composition of the transgene sequence of the rAAV vector will depend upon the use to which the resulting vector will be put. For example, one type of transgene sequence includes a reporter sequence, which upon expression produces a detectable signal. In another example, the transgene encodes a therapeutic protein or therapeutic functional RNA. In another example, the transgene encodes a protein or functional RNA that is intended to be used for research purposes, e.g., to create a somatic transgenic animal model harboring the transgene, e.g., to study the function of the transgene product. In another example, the transgene encodes a protein or functional RNA that is intended to be used to create an animal model of disease. Appropriate transgene coding sequences will be apparent to the skilled artisan.

Reporter sequences that may be provided in a transgene include, without limitation, DNA sequences encoding β-lactamase, β-galactosidase (LacZ), alkaline phosphatase, thymidine kinase, green fluorescent protein (e.g., GFP, EGFP), chloramphenicol acetyltransferase (CAT), luciferase (e.g., Firefly luciferase), and others well known in the art. When associated with regulatory elements which drive their expression, the reporter sequences, provide signals detectable by conventional means, including enzymatic, radiographic, colorimetric, fluorescence or other spectrographic assays, fluorescent activating cell sorting assays and immunological assays, including enzyme linked immunosorbent assay (ELISA), radioimmunoassay (RIA) and immunohistochemistry (IHC). For example, where the marker sequence is the LacZ gene, the presence of the vector carrying the signal is detected by assays for β-galactosidase activity. Where the transgene is green fluorescent protein or luciferase, the vector carrying the signal may be measured visually by color or light production in a luminometer. Such reporters can, for example, be useful in verifying the tissue-specific targeting capabilities and tissue specific promoter regulatory activity of an rAAV.

In some aspects, the disclosure provides rAAV vectors for use in methods of preventing or treating one or more genetic deficiencies or dysfunctions in a mammal, such as, for example, a polypeptide deficiency or polypeptide excess in a mammal, and particularly for treating or reducing the severity or extent of deficiency in a human manifesting one or more of the disorders linked to a deficiency in such polypeptides in cells and tissues. The method involves administration of an rAAV vector that encodes one or more gene products (e.g., therapeutic peptides, polypeptides, siRNAs, microRNAs, antisense nucleotides, etc.) in a pharmaceutically acceptable carrier to the subject in an amount and for a period of time sufficient to treat the deficiency or disorder in the subject suffering from such a disorder.

Thus, the disclosure embraces the delivery of rAAV vectors encoding one or more peptides, polypeptides, or proteins, which are useful for the treatment or prevention of disease states in a mammalian subject. Exemplary therapeutic proteins include one or more polypeptides selected from the group consisting of growth factors, interleukins, interferons, anti-apoptosis factors, cytokines, anti-diabetic factors, anti-apoptosis agents, coagulation factors, anti-tumor factors. Other non-limiting examples of therapeutic proteins include BDNF, CNTF, CSF, EGF, FGF, G-SCF, GM-CSF, gonadotropin, IFN, IFG-1, M-CSF, NGF, PDGF, PEDF, TGF, VEGF, TGF-B2, TNF, prolactin, somatotropin, XIAP1, IL-1, IL-2, IL-3, IL-4, IL-5, IL-6, IL-7, IL-8, IL-9, IL-10, IL-10 (187A), viral IL-10, IL-11, IL-12, IL-13, IL-14, IL-15, IL-16 IL-17, and IL-18.

In some embodiments, the disclosure involves the delivery of rAAV vectors encoding an anti-vascular endothelial growth factor (anti-VEGF) agent. In some embodiments, the anti-VEGF is KH902. Vascular endothelial growth factor (VEGF), originally known as vascular permeability factor (VPF), is a signal protein produced by cells that stimulates the formation of blood vessels. Aberrant VEGF activity/signaling contributes to various diseases, such as vascular diseases.

Anti-vascular endothelial growth factor therapy, also known as anti-VEGF therapy or anti-VEGF medication, is the use of medications that block vascular endothelial growth factor activity. Non-limiting examples of anti-VEGF agents include VEGF receptor fusion proteins (e.g., KH902), monoclonal antibodies such as bevacizumab, antibody derivatives such as ranibizumab (Lucentis), or orally available small molecules that inhibit the tyrosine kinases stimulated by VEGF (e.g., lapatinib, sunitinib, sorafenib, axitinib, and pazopanib). In some embodiments, the anti-VEGF agent (e.g., KH902) targets (e.g., specifically binds to) a human VEGF receptor. In some embodiments, an anti-VEGF agent targets (e.g., specifically binds to) a placental-derived growth factor (PlGF).

An exemplary coding sequence for KH902 is set forth in SEQ ID NO: 8.

(SEQ ID NO: 8)
ATGGTCAGCTACTGGGACACCGGGGTCCTGCTGTGCGCGCTGCTCAGCTGT
CTGCTTCTCACAGGATCTAGTTCCGGAGGTAGACCTTTCGTAGAGATGTA
CAGTGAAATCCCCGAAATTATACACATGACTGAAGGAAGGGAGCTCGTCA
TTCCCTGCCGGGTTACGTCACCTAACATCACTGTTACTTTAAAAAAGTTT
CCACTTGACACTTTGATCCCTGATGGAAAACGCATAATCTGGGACAGTAG
AAAGGGCTTCATCATATCAAATGCAACGTACAAAGAAATAGGGCTTCTGA
CCTGTGAAGCAACAGICAATGGGCATTTGTATAAGACAAACTATCTCACA
CATCGACAAACCAATACAATCATAGATGTGGTTCTGAGTCCGTCTCATGG
AATTGAACTATCTGTTGGAGAAAAGCTTGTCTTAAATTGTACAGCAAGAA
CTGAACTAAATGTGGGGATTGACTTCAACTGGGAATACCCTTCTTCGAAG
CATCAGCATAAGAAACTTGTAAACCGAGACCTAAAAACCCAGTCTGGGAG
TGAGATGAAGAAATTTTTGAGCACCTTAACTATAGATGGTGTAACCCGGA
GTGACCAAGGATTGTACACCTGTGCAGCATCCAGTGGGCTGATGACCAAG
AAGAACAGCACATTTGTCAGGGTCCATGAAAAACCTTTTGTTGCTTTTGG
AAGTGGCATGGAATCTCTGGTGGAAGCCACGGTGGGGGAGCGTGTCAGAA
TCCCTGCGAAGTACCTTGGTTACCCACCCCCAGAAATAAAATGGTATAAA
AATGGAATACCCCTTGAGTCCAATCACACAATTAAAGCGGGGCATGTACT
GACGATTATGGAAGTGAGTGAAAGAGACACAGGAAATTACACTGTCATCC
TTACCAATCCCATTTCAAAGGAGAAGCAGAGCCATGTGGTCTCTCTGGTT
GTGTATGTCCCACCGGGCCCGGGCGACAAAACTCACACATGCCCACTGTG
CCCAGCACCTGAACTCCTGGGGGGACCGTCAGTCTTCCTCTTCCCCCCAA
AACCCAAGGACACCCTCATGATCTCCCGGACCCCTGAGGTCACATGCGTG
GTGGTGGACGTGAGCCACGAAGACCCTGAGGTCAAGTTCAACTGGTACGT
GGACGGCGTGGAGGTGCATAATGCCAAGACAAAGCCGCGGGAGGAGCAGT
ACAACAGCACGTACCGTGTGGTCAGCGTCCTCACCGTCCTGCACCAGGAC
TGGCTGAATGGCAAGGAGTACAAGTGCAAGGTCTCCAACAAAGCCCTCCC
AGCCCCCATCGAGAAAACCATCTCCAAAGCCAAAGGGCAGCCCCGAGAAC
CACAGGTGTACACCCTGCCCCCATCCCGGGATGAGCTGACCAAGAACCAG
GTCAGCCTGACCTGCCTAGTCAAAGGCTTCTATCCCAGCGACATCGCCGT
GGAGTGGGAGAGCAATGGGCAGCCGGAGAACAACTACAAGGCCACGCCTC
CCGTGCTGGACTCCGACGGCTCCTTCTTCCTCTACAGCAAGCTCACCGTG
GACAAGAGCAGGTGGCAGCAGGGGAACGTCTTCTCATGCTCCGTGATGCA
TGAGGCTCTGCACAACCACTACACGCAGAAGAGCCTCTCCCTGTCTCCGG
GTAAATGA

The rAAV vectors may comprise a gene to be transferred to a subject to treat a disease associated with reduced expression, lack of expression or dysfunction of the gene. Exemplary genes and associated disease states include, but are not limited to: glucose-6-phosphatase, associated with glycogen storage deficiency type 1A; phosphoenolpyruvate-carboxykinase, associated with Pepck deficiency; galactose-1 phosphate uridyl transferase, associated with galactosemia; phenylalanine hydroxylase, associated with phenylketonuria; branched chain alpha-ketoacid dehydrogenase, associated with Maple syrup urine disease; fumarylacetoacetate hydrolase, associated with tyrosinemia type 1; methylmalonyl-CoA mutase, associated with methylmalonic acidemia; medium chain acyl CoA dehydrogenase, associated with medium chain acetyl CoA deficiency; omithine transcarbamylase, associated with omithine transcarbamylase deficiency; argininosuccinic acid synthetase, associated with citrullinemia; low density lipoprotein receptor protein, associated with familial hypercholesterolemia; UDP-glucouronosyltransferase, associated with Crigler-Najjar disease; adenosine deaminase, associated with severe combined immunodeficiency disease; hypoxanthine guanine phosphoribosyl transferase, associated with Gout and Lesch-Nyan syndrome; biotinidase, associated with biotinidase deficiency; beta-glucocerebrosidase, associated with Gaucher disease; beta-glucuronidase, associated with Sly syndrome; peroxisome membrane protein 70 kDa, associated with Zellweger syndrome; porphobilinogen deaminase, associated with acute intermittent porphyria; alpha-1 antitrypsin for treatment of alpha-1 antitrypsin deficiency (emphysema); erythropoietin for treatment of anemia due to thalassemia or to renal failure; vascular endothelial growth factor, angiopoietin-1, and fibroblast growth factor for the treatment of ischemic diseases; thrombomodulin and tissue factor pathway inhibitor for the treatment of occluded blood vessels as seen in, for example, atherosclerosis, thrombosis, or embolisms; aromatic amino acid decarboxylase (AADC), and tyrosine hydroxylase (TH) for the treatment of Parkinson's disease; the beta adrenergic receptor, anti-sense to, or a mutant form of, phospholamban, the sarcoendoplasmic reticulum adenosine triphosphatase-2 (SERCA2), and the cardiac adenylyl cyclase for the treatment of congestive heart failure; a tumor suppressor gene such as p53 for the treatment of various cancers; a cytokine such as one of the various interleukins for the treatment of inflammatory and immune disorders and cancers; dystrophin or minidystrophin and utrophin or miniutrophin for the treatment of muscular dystrophies; and, insulin for the treatment of diabetes.

The skilled artisan will also realize that in the case of transgenes encoding proteins or polypeptides, mutations that result in conservative amino acid substitutions may be made in a transgene to provide functionally equivalent variants or homologs of a protein or polypeptide. In some aspects the disclosure embraces sequence alterations that result in conservative amino acid substitution of a transgene. In some embodiments, the transgene comprises a gene having a dominant negative mutation. For example, a transgene may express a mutant protein that interacts with the same elements as a wild-type protein, and thereby blocks some aspect of the function of the wild-type protein.

Useful transgene products also include miRNAs. miRNAs and other small interfering nucleic acids regulate gene expression via target RNA transcript cleavage/degradation or translational repression of the target messenger RNA (mRNA). miRNAs are natively expressed, typically as final 19-25 non-translated RNA products. miRNAs exhibit their activity through sequence-specific interactions with the 3′ untranslated regions (UTR) of target mRNAs. These endogenously expressed miRNAs form hairpin precursors that are subsequently processed into a miRNA duplex, and further into a “mature” single stranded miRNA molecule. This mature miRNA guides a multiprotein complex, miRISC, which identifies target sites, e.g., in the 3′ UTR regions, of target mRNAs based upon their complementarity to the mature miRNA.

The following non-limiting list of miRNA genes, and their homologues, are useful as transgenes or as targets for small interfering nucleic acids encoded by transgenes (e.g., miRNA sponges, antisense oligonucleotides, TuD RNAs) in certain embodiments of the methods: hsa-let-7a, hsa-let-7a*, hsa-let-7b, hsa-let-7b*, hsa-let-7c, hsa-let-7c*, hsa-let-7d, hsa-let-7d*, hsa-let-7e, hsa-let-7e*, hsa-let-7f, hsa-let-7f-1*, hsa-let-7f-2*, hsa-let-7g, hsa-let-7g*, hsa-let-7i, hsa-let-7i*, hsa-miR-1, hsa-miR-100, hsa-miR-100*, hsa-miR-101, hsa-miR-101*, hsa-miR-103, hsa-miR-105, hsa-miR-105*, hsa-miR-106a, hsa-miR-106a*, hsa-miR-106b, hsa-miR-106b*, hsa-miR-107, hsa-miR-10a, hsa-miR-10a*, hsa-miR-10b, hsa-miR-10b*, hsa-miR-1178, hsa-miR-1179, hsa-miR-1180, hsa-miR-1181, hsa-miR-1182, hsa-miR-1183, hsa-miR-1184, hsa-miR-1185, hsa-miR-1197, hsa-miR-1200, hsa-miR-1201, hsa-miR-1202, hsa-miR-1203, hsa-miR-1204, hsa-miR-1205, hsa-miR-1206, hsa-miR-1207-3p, hsa-miR-1207-5p, hsa-miR-1208, hsa-miR-122, hsa-miR-122*, hsa-miR-1224-3p, hsa-miR-1224-5p, hsa-miR-1225-3p, hsa-miR-1225-5p, hsa-miR-1226, hsa-miR-1226*, hsa-miR-1227, hsa-miR-1228, hsa-miR-1228*, hsa-miR-1229, hsa-miR-1231, hsa-miR-1233, hsa-miR-1234, hsa-miR-1236, hsa-miR-1237, hsa-miR-1238, hsa-miR-124, hsa-miR-124*, hsa-miR-1243, hsa-miR-1244, hsa-miR-1245, hsa-miR-1246, hsa-miR-1247, hsa-miR-1248, hsa-miR-1249, hsa-miR-1250, hsa-miR-1251, hsa-miR-1252, hsa-miR-1253, hsa-miR-1254, hsa-miR-1255a, hsa-miR-1255b, hsa-miR-1256, hsa-miR-1257, hsa-miR-1258, hsa-miR-1259, hsa-miR-125a-3p, hsa-miR-125a-5p, hsa-miR-125b, hsa-miR-125b-1*, hsa-miR-125b-2*, hsa-miR-126, hsa-miR-126*, hsa-miR-1260, hsa-miR-1261, hsa-miR-1262, hsa-miR-1263, hsa-miR-1264, hsa-miR-1265, hsa-miR-1266, hsa-miR-1267, hsa-miR-1268, hsa-miR-1269, hsa-miR-1270, hsa-miR-1271, hsa-miR-1272, hsa-miR-1273, hsa-miR-127-3p, hsa-miR-1274a, hsa-miR-1274b, hsa-miR-1275, hsa-miR-127-5p, hsa-miR-1276, hsa-miR-1277, hsa-miR-1278, hsa-miR-1279, hsa-miR-128, hsa-miR-1280, hsa-miR-1281, hsa-miR-1282, hsa-miR-1283, hsa-miR-1284, hsa-miR-1285, hsa-miR-1286, hsa-miR-1287, hsa-miR-1288, hsa-miR-1289, hsa-miR-129*, hsa-miR-1290, hsa-miR-1291, hsa-miR-1292, hsa-miR-1293, hsa-miR-129-3p, hsa-miR-1294, hsa-miR-1295, hsa-miR-129-5p, hsa-miR-1296, hsa-miR-1297, hsa-miR-1298, hsa-miR-1299, hsa-miR-1300, hsa-miR-1301, hsa-miR-1302, hsa-miR-1303, hsa-miR-1304, hsa-miR-1305, hsa-miR-1306, hsa-miR-1307, hsa-miR-1308, hsa-miR-130a, hsa-miR-130a*, hsa-miR-130b, hsa-miR-130b*, hsa-miR-132, hsa-miR-132*, hsa-miR-1321, hsa-miR-1322, hsa-miR-1323, hsa-miR-1324, hsa-miR-133a, hsa-miR-133b, hsa-miR-134, hsa-miR-135a, hsa-miR-135a*, hsa-miR-135b, hsa-miR-135b*, hsa-miR-136, h isa-miR-13, hsa-miR-137, hsa-miR-138, hsa-miR-138-1*, hsa-miR-138-2*, hsa-miR-139-3p, hsa-miR-139-5p, hsa-miR-140-3p, hsa-miR-140-5p, hsa-miR-141, hsa-miR-141*, hsa-miR-142-3p, hsa-miR-142-5p, hsa-miR-143, hsa-miR-143*, hsa-miR-144, hsa-miR-144*, hsa-miR-145, hsa-miR-145*, hsa-miR-146a, hsa-miR-146a*, hsa-miR-146b-3p, hsa-miR-146b-5p, hsa-miR-147, hsa-miR-147b, hsa-miR-148a, hsa-miR-148a*, hsa-miR-148b, hsa-miR-148b*, hsa-miR-149, hsa-miR-149*, hsa-miR-150, hsa-miR-150*, hsa-miR-151-3p, hsa-miR-151-5p, hsa-miR-152, hsa-miR-153, hsa-miR-154, hsa-miR-154*, hsa-miR-155, hsa-miR-155*, hsa-miR-15a, hsa-miR-15a*, hsa-miR-15b, hsa-miR-15b*, hsa-miR-16, hsa-miR-16-1*, hsa-miR-16-2*, hsa-miR-17, hsa-miR-17*, hsa-miR-181a, hsa-miR-181a*, hsa-miR-181a-2*, hsa-miR-181b, hsa-miR-181c, hsa-miR-181c*, hsa-miR-181d, hsa-miR-182, hsa-miR-182*, hsa-miR-1825, hsa-miR-1826, hsa-miR-1827, hsa-miR-183, hsa-miR-183*, hsa-miR-184, hsa-miR-185, hsa-miR-185*, hsa-miR-186, hsa-miR-186*, hsa-miR-187, hsa-miR-187*, hsa-miR-188-3p, hsa-miR-188-5p, hsa-miR-18a, hsa-miR-18a*, hsa-miR-18b, hsa-miR-18b*, hsa-miR-190, hsa-miR-190b, hsa-miR-191, hsa-miR-191*, hsa-miR-192, hsa-miR-192*, hsa-miR-193a-3p, hsa-miR-193a-5p, hsa-miR-193b, hsa-miR-193b*, hsa-miR-194, hsa-miR-194*, hsa-miR-195, hsa-miR-195*, hsa-miR-196a, hsa-miR-196a*, hsa-miR-196b, hsa-miR-197, hsa-miR-198, hsa-miR-199a-3p, hsa-miR-199a-5p, hsa-miR-199b-5p, hsa-miR-19a, hsa-miR-19a*, hsa-miR-19b, hsa-miR-19b-1*, hsa-miR-19b-2*, hsa-miR-200a, hsa-miR-200a*, hsa-miR-200b, hsa-miR-200b*, hsa-miR-200c, hsa-miR-200c*, hsa-miR-202, hsa-miR-202*, hsa-miR-203, hsa-miR-204, hsa-miR-205, hsa-miR-206, hsa-miR-208a, hsa-miR-208b, hsa-miR-20a, hsa-miR-20a*, hsa-miR-20b, hsa-miR-20b*, hsa-miR-21, hsa-miR-21*, hsa-miR-210, hsa-miR-211, hsa-miR-212, hsa-miR-214, hsa-miR-214*, hsa-miR-215, hsa-miR-216a, hsa-miR-216b, hsa-miR-217, hsa-miR-218, hsa-miR-218-1*, hsa-miR-218-2*, hsa-miR-219-1-3p, hsa-miR-219-2-3p, hsa-miR-219-5p, hsa-miR-22, hsa-miR-22*, hsa-miR-220a, hsa-miR-220b, hsa-miR-220c, hsa-miR-221, hsa-miR-221*, hsa-miR-222, hsa-miR-222*, hsa-miR-223, hsa-miR-223*, hsa-miR-224, hsa-miR-23a, hsa-miR-23a*, hsa-miR-23b, hsa-miR-23b*, hsa-miR-24, hsa-miR-24-1*, hsa-miR-24-2*, hsa-miR-25, hsa-miR-25*, hsa-miR-26a, hsa-miR-26a-1*, hsa-miR-26a-2*, hsa-miR-26b, hsa-miR-26b*, hsa-miR-27a, hsa-miR-27a*, hsa-miR-27b, hsa-miR-27b*, hsa-miR-28-3p, hsa-miR-28-5p, hsa-miR-296-3p, hsa-miR-296-5p, hsa-miR-297, hsa-miR-298, hsa-miR-299-3p, hsa-miR-299-5p, hsa-miR-29a, hsa-miR-29a*, hsa-miR-29b, hsa-miR-29b-1*, hsa-miR-29b-2*, hsa-miR-29c, hsa-miR-29c*, hsa-miR-300, hsa-miR-301a, hsa-miR-301b, hsa-miR-302a, hsa-miR-302a*, hsa-miR-302b, hsa-miR-302b*, hsa-miR-302c, hsa-miR-302c*, hsa-miR-302d, hsa-miR-302d*, hsa-miR-302e, hsa-miR-302f, hsa-miR-30a, hsa-miR-30a*, hsa-miR-30b, hsa-miR-30b*, hsa-miR-30c, hsa-miR-30c-1*, hsa-miR-30c-2*, hsa-miR-30d, hsa-miR-30d*, hsa-miR-30e, hsa-miR-30e*, hsa-miR-31, hsa-miR-31*, hsa-miR-32, hsa-miR-32*, hsa-miR-320a, hsa-miR-320b, hsa-miR-320c, hsa-miR-320d, hsa-miR-323-3p, hsa-miR-323-5p, hsa-miR-324-3p, hsa-miR-324-5p, hsa-miR-325, hsa-miR-326, hsa-miR-328, hsa-miR-329, hsa-miR-330-3p, hsa-miR-330-5p, hsa-miR-331-3p, hsa-miR-331-5p, hsa-miR-335, hsa-miR-335*, hsa-miR-337-3p, hsa-miR-337-5p, hsa-miR-338-3p, hsa-miR-338-5p, hsa-miR-339-3p, hsa-miR-339-5p, hsa-miR-33a, hsa-miR-33a*, hsa-miR-33b, hsa-miR-33b*, hsa-miR-340, hsa-miR-340*, hsa-miR-342-3p, hsa-miR-342-5p, hsa-miR-345, hsa-miR-346, hsa-miR-34a, hsa-miR-34a*, hsa-miR-34b, hsa-miR-34b*, hsa-miR-34c-3p, hsa-miR-34c-5p, hsa-miR-361-3p, hsa-miR-361-5p, hsa-miR-362-3p, hsa-miR-362-5p, hsa-miR-363, hsa-miR-363*, hsa-miR-365, hsa-miR-367, hsa-miR-367*, hsa-miR-369-3p, hsa-miR-369-5p, hsa-miR-370, hsa-miR-371-3p, hsa-miR-371-5p, hsa-miR-372, hsa-miR-373, hsa-miR-373*, hsa-miR-374a, hsa-miR-374a*, hsa-miR-374b, hsa-miR-374b*, hsa-miR-375, hsa-miR-376a, hsa-miR-376a*, hsa-miR-376b, hsa-miR-376c, hsa-miR-377, hsa-miR-377*, hsa-miR-378, hsa-miR-378*, hsa-miR-379, hsa-miR-379*, hsa-miR-380, hsa-miR-380*, hsa-miR-381, hsa-miR-382, hsa-miR-383, hsa-miR-384, hsa-miR-409-3p, hsa-miR-409-5p, hsa-miR-410, hsa-miR-411, hsa-miR-411*, hsa-miR-412, hsa-miR-421, hsa-miR-422a, hsa-miR-423-3p, hsa-miR-423-5p, hsa-miR-424, hsa-miR-424*, hsa-miR-425, hsa-miR-425*, hsa-miR-429, hsa-miR-431, hsa-miR-431*, hsa-miR-432, hsa-miR-432*, hsa-miR-433, hsa-miR-448, hsa-miR-449a, hsa-miR-449b, hsa-miR-450a, hsa-miR-450b-3p, hsa-miR-450b-5p, hsa-miR-451, hsa-miR-452, hsa-miR-452*, hsa-miR-453, hsa-miR-454, hsa-miR-454*, hsa-miR-455-3p, hsa-miR-455-5p, hsa-miR-483-3p, hsa-miR-483-5p, hsa-miR-484, hsa-miR-485-3p, hsa-miR-485-5p, hsa-miR-486-3p, hsa-miR-486-5p, hsa-miR-487a, hsa-miR-487b, hsa-miR-488, hsa-miR-488*, hsa-miR-489, hsa-miR-490-3p, hsa-miR-490-5p, hsa-miR-491-3p, hsa-miR-491-5p, hsa-miR-492, hsa-miR-493, hsa-miR-493*, hsa-miR-494, hsa-miR-495, hsa-miR-496, hsa-miR-497, hsa-miR-497*, hsa-miR-498, hsa-miR-499-3p, hsa-miR-499-5p, hsa-miR-500, hsa-miR-500*, hsa-miR-501-3p, hsa-miR-501-5p, hsa-miR-502-3p, hsa-miR-502-5p, hsa-miR-503, hsa-miR-504, hsa-miR-505, hsa-miR-505*, hsa-miR-506, hsa-miR-507, hsa-miR-508-3p, hsa-miR-508-5p, hsa-miR-509-3-5p, hsa-miR-509-3p, hsa-miR-509-5p, hsa-miR-510, hsa-miR-511, hsa-miR-512-3p, hsa-miR-512-5p, hsa-miR-513a-3p, hsa-miR-513a-5p, hsa-miR-513b, hsa-miR-513c, hsa-miR-514, hsa-miR-515-3p, hsa-miR-515-5p, hsa-miR-516a-3p, hsa-miR-516a-5p, hsa-miR-516b, hsa-miR-517*, hsa-miR-517a, hsa-miR-517b, hsa-miR-517c, hsa-miR-518a-3p, hsa-miR-518a-5p, hsa-miR-518b, hsa-miR-518c, hsa-miR-518c*, hsa-miR-518d-3p, hsa-miR-518d-5p, hsa-miR-518e, hsa-miR-518e*, hsa-miR-518f, hsa-miR-518f*, hsa-miR-519a, hsa-miR-519b-3p, hsa-miR-519c-3p, hsa-miR-519d, hsa-miR-519e, hsa-miR-519e*, hsa-miR-520a-3p, hsa-miR-520a-5p, hsa-miR-520b, hsa-miR-520c-3p, hsa-miR-520d-3p, hsa-miR-520d-5p, hsa-miR-520e, hsa-miR-520f, hsa-miR-520g, hsa-miR-520h, hsa-miR-521, hsa-miR-522, hsa-miR-523, hsa-miR-524-3p, hsa-miR-524-5p, hsa-miR-525-3p, hsa-miR-525-5p, hsa-miR-526b, hsa-miR-526b*, hsa-miR-532-3p, hsa-miR-532-5p, hsa-miR-539, hsa-miR-541, hsa-miR-541*, hsa-miR-542-3p, hsa-miR-542-5p, hsa-miR-543, hsa-miR-544, hsa-miR-545, hsa-miR-545*, hsa-miR-548a-3p, hsa-miR-548a-5p, hsa-miR-548b-3p, hsa-miR-548b-5p, hsa-miR-548c-3p, hsa-miR-548c-5p, hsa-miR-548d-3p, hsa-miR-548d-5p, hsa-miR-548e, hsa-miR-548f, hsa-miR-548g, hsa-miR-548h, hsa-miR-548i, hsa-miR-548j, hsa-miR-548k, hsa-miR-548l, hsa-miR-548m, hsa-miR-548n, hsa-miR-548o, hsa-miR-548p, hsa-miR-549, hsa-miR-550, hsa-miR-550*, hsa-miR-551a, hsa-miR-551b, hsa-miR-551b*, hsa-miR-552, hsa-miR-553, hsa-miR-554, hsa-miR-555, hsa-miR-556-3p, hsa-miR-556-5p, hsa-miR-557, hsa-miR-558, hsa-miR-559, hsa-miR-561, hsa-miR-562, hsa-miR-563, hsa-miR-564, hsa-miR-566, hsa-miR-567, hsa-miR-568, hsa-miR-569, hsa-miR-570, hsa-miR-571, hsa-miR-572, hsa-miR-573, hsa-miR-574-3p, hsa-miR-574-5p, hsa-miR-575, hsa-miR-576-3p, hsa-miR-576-5p, hsa-miR-577, hsa-miR-578, hsa-miR-579, hsa-miR-580, hsa-miR-581, hsa-miR-582-3p, hsa-miR-582-5p, hsa-miR-583, hsa-miR-584, hsa-miR-585, hsa-miR-586, hsa-miR-587, hsa-miR-588, hsa-miR-589, hsa-miR-589*, hsa-miR-590-3p, hsa-miR-590-5p, hsa-miR-591, hsa-miR-592, hsa-miR-593, hsa-miR-593*, hsa-miR-595, hsa-miR-596, hsa-miR-597, hsa-miR-598, hsa-miR-599, hsa-miR-600, hsa-miR-601, hsa-miR-602, hsa-miR-603, hsa-miR-604, hsa-miR-605, hsa-miR-606, hsa-miR-607, hsa-miR-608, hsa-miR-609, hsa-miR-610, hsa-miR-611, hsa-miR-612, hsa-miR-613, hsa-miR-614, hsa-miR-615-3p, hsa-miR-615-5p, hsa-miR-616, hsa-miR-616*, hsa-miR-617, hsa-miR-618, hsa-miR-619, hsa-miR-620, hsa-miR-621, hsa-miR-622, hsa-miR-623, hsa-miR-624, hsa-miR-624*, hsa-miR-625, hsa-miR-625*, hsa-miR-626, hsa-miR-627, hsa-miR-628-3p, hsa-miR-628-5p, hsa-miR-629, hsa-miR-629*, hsa-miR-630, hsa-miR-631, hsa-miR-632, hsa-miR-633, hsa-miR-634, hsa-miR-635, hsa-miR-636, hsa-miR-637, hsa-miR-638, hsa-miR-639, hsa-miR-640, hsa-miR-641, hsa-miR-642, hsa-miR-643, hsa-miR-644, hsa-miR-645, hsa-miR-646, hsa-miR-647, hsa-miR-648, hsa-miR-649, hsa-miR-650, hsa-miR-651, hsa-miR-652, hsa-miR-653, hsa-miR-654-3p, hsa-miR-654-5p, hsa-miR-655, hsa-miR-656, hsa-miR-657, hsa-miR-658, hsa-miR-659, hsa-miR-660, hsa-miR-661, hsa-miR-662, hsa-miR-663, hsa-miR-663b, hsa-miR-664, hsa-miR-664*, hsa-miR-665, hsa-miR-668, hsa-miR-671-3p, hsa-miR-671-5p, hsa-miR-675, hsa-miR-7, hsa-miR-708, hsa-miR-708*, hsa-miR-7-1*, hsa-miR-7-2*, hsa-miR-720, hsa-miR-744, hsa-miR-744*, hsa-miR-758, hsa-miR-760, hsa-miR-765, hsa-miR-766, hsa-miR-767-3p, hsa-miR-767-5p, hsa-miR-768-3p, hsa-miR-768-5p, hsa-miR-769-3p, hsa-miR-769-5p, hsa-miR-770-5p, hsa-miR-802, hsa-miR-873, hsa-miR-874, hsa-miR-875-3p, hsa-miR-875-5p, hsa-miR-876-3p, hsa-miR-876-5p, hsa-miR-877, hsa-miR-877*, hsa-miR-885-3p, hsa-miR-885-5p, hsa-miR-886-3p, hsa-miR-886-5p, hsa-miR-887, hsa-miR-888, hsa-miR-888*, hsa-miR-889, hsa-miR-890, hsa-miR-891a, hsa-miR-891b, hsa-miR-892a, hsa-miR-892b, hsa-miR-9, hsa-miR-9*, hsa-miR-920, hsa-miR-921, hsa-miR-922, hsa-miR-923, hsa-miR-924, hsa-miR-92a, hsa-miR-92a-1*, hsa-miR-92a-2*, hsa-miR-92b, hsa-miR-92b*, hsa-miR-93, hsa-miR-93*, hsa-miR-933, hsa-miR-934, hsa-miR-935, hsa-miR-936, hsa-miR-937, hsa-miR-938, hsa-miR-939, hsa-miR-940, hsa-miR-941, hsa-miR-942, hsa-miR-943, hsa-miR-944, hsa-miR-95, hsa-miR-96, hsa-miR-96*, hsa-miR-98, hsa-miR-99a, hsa-miR-99a*, hsa-miR-99b, and hsa-miR-99b*.

A miRNA leads to degradation of the mRNAs it targets and, as a result, inhibits expression of the polypeptides encoded by the mRNAs. Thus, blocking (partially or totally) the presence of the miRNA (e.g., silencing the miRNA) can effectively induce, or restore, expression of a polypeptide whose expression is inhibited (derepress the polypeptide). In one embodiment, derepression of polypeptides encoded by mRNA targets of a miRNA is accomplished by inhibiting the miRNA activity in cells through any one of a variety of methods. For example, blocking the activity of a miRNA can be accomplished by hybridization with a small interfering nucleic acid (e.g., antisense oligonucleotide, miRNA sponge, TuD RNA) that is complementary, or substantially complementary to, the miRNA, thereby blocking interaction of the miRNA with its target mRNA. As used herein, a small interfering nucleic acid that is substantially complementary to a miRNA is one that is capable of hybridizing with a miRNA, and blocking the miRNA's activity. In some embodiments, a small interfering nucleic acid that is substantially complementary to a miRNA is a small interfering nucleic acid that is complementary to the miRNA at all but 1, 2, 3, 4, 5, 6, 7, 8, 9, 10, 11, 12, 13, 14, 15, 16, 17, or 18 bases. In some embodiments, a small interfering nucleic acid sequence that is substantially complementary to a miRNA, or is a small interfering nucleic acid sequence that is complementary to the miRNA with at least one base.

A “miRNA Inhibitor” is an agent that blocks miRNA function, expression and/or processing. For instance, these molecules include, but are not limited to, microRNA specific antisense oligonucleotides, microRNA sponges, tough decoy RNAs (TuD RNAs), and microRNA oligonucleotides (double-stranded, hairpin, short oligonucleotides) that inhibit miRNA interaction with a Drosha complex. MicroRNA inhibitors can be expressed in cells from a transgene of a rAAV vector, as discussed above. MicroRNA sponges specifically inhibit miRNAs through a complementary heptameric seed sequence (Ebert, M. S. Nature Methods, Epub Aug. 12, 2007). In some embodiments, an entire family of miRNAs can be silenced using a single sponge sequence. TuD RNAs achieve efficient and long-term-suppression of specific miRNAs in mammalian cells (See, e.g., Takeshi Haraguchi, et al., Nucleic Acids Research, 2009, Vol. 37, No. 6 e43, the contents of which relating to TuD RNAs are incorporated herein by reference). Other methods for silencing miRNA function (derepression of miRNA targets) in cells will be apparent to one of ordinary skill in the art.

In some embodiments, the cloning capacity of the recombinant RNA vector may limit a desired coding sequence and may require the complete replacement of the virus's 4.8 kilobase genome. Large genes may, therefore, not be suitable for use in a standard recombinant AAV vector, in some cases. The skilled artisan will appreciate that options are available in the art for overcoming a limited coding capacity. For example, the AAV ITRs of two genomes can anneal to form head to tail concatamers, almost doubling the capacity of the vector. Insertion of splice sites allows for the removal of the ITRs from the transcript. Other options for overcoming a limited cloning capacity will be apparent to the skilled artisan.

### Administration

The rAAVs may be delivered to a subject in compositions according to any appropriate methods known in the art. The rAAV, preferably suspended in a physiologically compatible carrier (e.g., in a composition), may be administered to a subject, e.g., host animal, such as a human, mouse, rat, cat, dog, sheep, rabbit, horse, cow, goat, pig, guinea pig, hamster, chicken, turkey, or a non-human primate (e.g., Macaque). In some embodiments a host animal does not include a human.

Delivery of the rAAVs to a mammalian subject may be by, for example, intramuscular injection or by administration into the bloodstream of the mammalian subject. In some embodiments, an rAAV is delivered to the eye of a subject through intravitreal injection. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue. Moreover, in certain instances, it may be desirable to deliver the virions to the CNS of a subject. By “CNS” is meant all cells and tissue of the brain and spinal cord of a vertebrate. Thus, the term includes, but is not limited to, neuronal cells, glial cells, astrocytes, cerebrospinal fluid (CSF), interstitial spaces, bone, cartilage and the like. Recombinant AAVs may be delivered directly to the CNS or brain by injection into, e.g., the ventricular region, as well as to the striatum (e.g., the caudate nucleus or putamen of the striatum), spinal cord and neuromuscular junction, or cerebellar lobule, with a needle, catheter or related device, using neurosurgical techniques known in the art, such as by stereotactic injection (see, e.g., Stein et al., J Virol 73:3424-3429, 1999; Davidson et al., PNAS 97:3428-3432, 2000; Davidson et al., Nat. Genet. 3:219-223, 1993; and Alisky and Davidson, Hum. Gene Ther. 11:2315-2329, 2000).

The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

The rAAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., intraportal delivery to the liver), oral, inhalation (including intranasal and intratracheal delivery), intraocular, intravenous, intramuscular, subcutaneous, intradermal, intratumoral, intracranial (e.g., intrahippocampal), and other parental routes of administration. Routes of administration may be combined, if desired.

In some embodiments, the rAAVs are administered directly to the eye by an ocular tissue injection such as periocular, conjunctival, subtenon, intracameral, intravitreal, intraocular, anterior or posterior juxtascleral, subretinal, subconjunctival, retrobulbar, or intracanalicular injections; by direct application to the eye using a catheter or other placement device such as a retinal pellet, intraocular insert, suppository or an implant comprising a porous, non-porous, or gelatinous material; by topical ocular drops or ointments; or by a slow release device in the cul-de-sac or implanted adjacent to the sclera (transscleral) or in the sclera (intrascleral) or within the eye. Intracameral injection may be through the cornea into the anterior chamber to allow the agent to reach the trabecular meshwork. Intracanalicular injection may be into the venous collector channels draining Schlemm's canal or into Schlemm's canal.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine a rAAV virion dose range to treat a patient having a particular disease or disorder based on the aforementioned factors, as well as other factors that are well known in the art. An effective amount of an rAAV is an amount sufficient to target infect an animal or target a desired tissue (e.g., ocular tissue). In some embodiments, an effective amount of an rAAV is an amount sufficient to produce a stable somatic transgenic animal model. The effective amount will depend primarily on factors such as the species, age, weight, health of the subject, and the tissue to be targeted, and may thus vary between animals or tissues. For example, an effective amount of the rAAV is generally in the range of from about 1 ml to about 100 ml of solution containing from about 10⁷ to 10¹⁶ genome copies. In some embodiments the rAAV is administered at a dose of 10⁷, 10⁸, 10⁹, 10¹⁰, 10¹¹, 10¹², 10¹³, 10¹⁴, or 10¹⁵ genome copies per subject. In some embodiments the rAAV is administered at a dose of 10⁷, 10⁸, 10⁹, 10¹⁰, 10¹¹, 10¹², 10¹³, 10¹⁴, or 10¹⁵ genome copies per kg. In some cases, a dosage between about 10¹¹ to 10¹² rAAV genome copies is appropriate. In some embodiments the rAAV is administered to the eye (e.g., by intravitreal injection) at a dose of about 10⁹ to 10¹⁶ genome copies per eye. In some embodiments, the rAAV is administered to the eye (e.g., by intravitreal injection) at a dose of about 10⁹, 10¹⁰, 10¹¹, 10¹², 10¹³, 10¹⁴, 10¹⁵, or 10¹⁶ genome copies per eye. In some embodiments, the rAAV is administered to the eye (e.g., by intravitreal injection) at a dose of 1×10⁹ genome copies per eye. In some embodiments, the rAAV is administered to the eye (e.g., by intravitreal injection) at a dose of 2.5×10⁸ genome copies per eye. In some cases, stable transgenic animals are produced by multiple doses of an rAAV.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well-known in the art and, include, for example, addition of surfactants, pH adjustment, salt concentration adjustment, etc. (See, e.g., Wright F R, et al., Molecular Therapy (2005) 12, 171-178, the contents of which are incorporated herein by reference.)

Formulation of pharmaceutically-acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intraopancreatically, intranasally, parenterally, intravenously, intracranially (e.g., intrahippocampally), intramuscularly, intrathecally, or orally, intraperitoneally, or by inhalation. In some embodiments, the administration modalities as described in U.S. Pat. Nos. 5,543,158; 5,641,515 and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof.

The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500.ANG., containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (i.e., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

### EXAMPLES

### Example 1. Rational Design of an AAV2 MC1 Capsid Protein for Targeting Ocular Cells

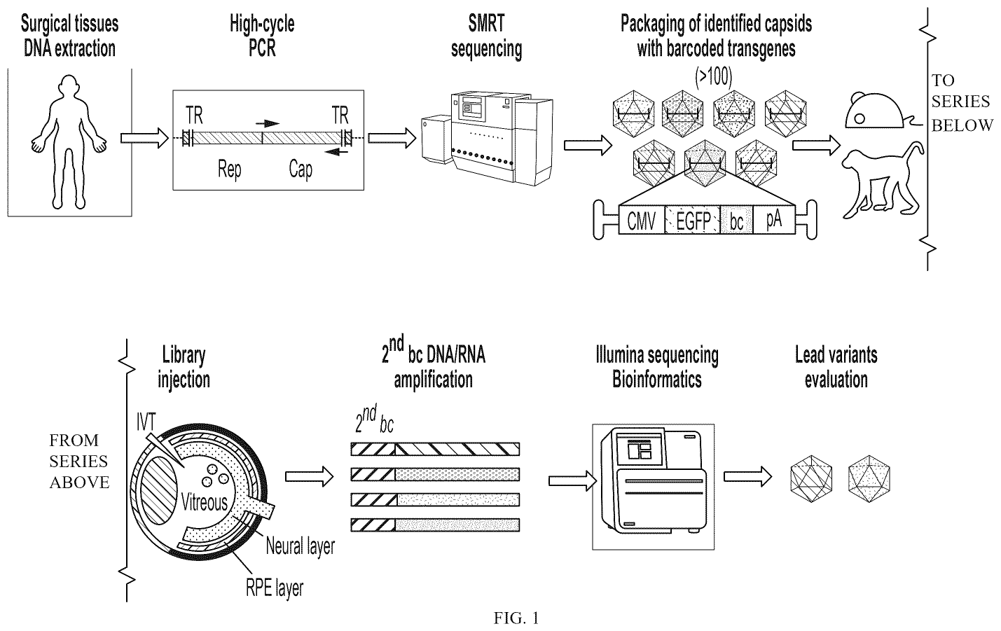

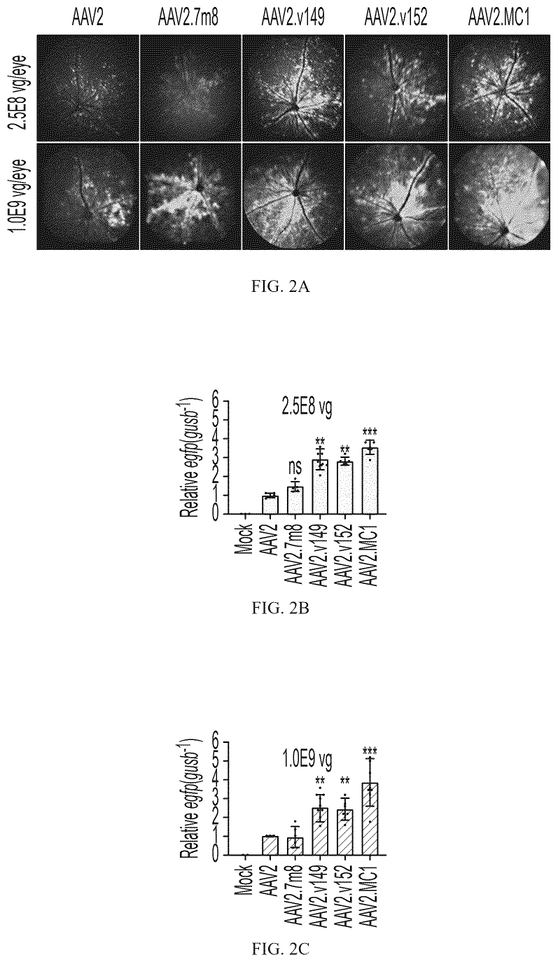

Several AAV2 capsid protein variants were identified as having specific ability for targeting ocular cells through the method shown in FIG. 1. Two human tissue-derived AAV2 variants, AAV2.v149 and AAV2.v152, were identified via mouse and non-human primate screens to identify capsids that can transduce retinal tissues (e.g., via intravitreal injection). rAAVs having AAV.v149 (SEQ ID NO: 2) and AAV.v152 (SEQ ID NO: 3) capsid proteins were found to confer more than two-fold better transduction of the mouse retina compared to rAAVs having AAV2 or AAV2.7m8 capsid proteins (FIGS. 2A-2C). AAV.v149 and AAV.v152 were found to have amino acid substitutions relative to wild-type AAV2 capsid protein (SEQ ID NO: 1); the specific amino acid substitutions are shown in Table 2. AAV.v149 and AAV.v152 have the same amino acid composition within the VP3 region as wild-type AAV2, and contain residue differences only within the VP1 unique (VP1u) region.

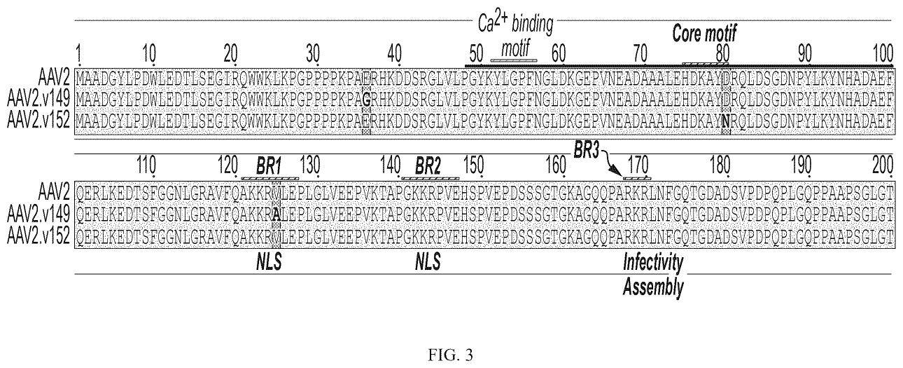

As shown in FIG. 3, the VP1u region has been reported to contain an essential phospholipase A2 (PLA2) domain, which includes a calcium-binding domain and a core enzymatic motif. The VP1u region also contains three basic regions (BR1-3), two of which overlap with nuclear localization sequences (NLS) (Girod et al., J Gen Virol. 2002; 83(Pt 5):973-8; Kurian et al. Viruses. 2019; 11(5); Johnson et al. J Virol. 2010; 84(17):8888-902). FIG. 3 also highlights the residues within the VP1u region that differ between wild-type AAV2 and AAV.v149 (E36G and V125A) and AAV.v152 (D80N). Notably, N80 is located within the PLA2 core enzymatic motif, and A125 is located within BR1.

Rational design of a VP1u region of a capsid protein harboring features of both AAV.v149 and AAV.v152 was carried out to generate a capsid protein having the E36G, D80N, and V125A substitution mutations, which was then named MCi. An AAV2 capsid protein having MCi within its VP1u region (AAV2.MC1) did not significantly alter packaging of single-strand or self-complementary AAV vectors (Table 3), and showed better transduction efficiency with higher doses (e.g., 1E9 vg/eye) compared to AAV2.v149 or AAV2.v159 (FIGS. 2A-2C).

TABLE 2
AAV2 Variant Amino Acid Substitutions

| AAV2.v149 | AAV2.v152 |
|---|---|
| E36G | |
| | D80N |
| V125A | |

TABLE 3
Titers achieved by human-derived capsids and AAV2.MC1

| | | Packaging yield |
|---|---|---|
| Name | Titer (gc/mL) | (% AAV2) |
| scAAV2.CB6-PI-Egfp | 4.12E12 | 100% |
| scAAV2.v149-CB6-PI-Egfp | 5.78E12 | 140% |
| scAAV2.v152-CB6-PI-Egfp | 5.48E12 | 133% |
| scAAV2.MC1-CB6-PI-Egfp | 2.20E12 | 53% |
| ssAAV2.CB6-PI-Fluc | 6.18E12 | 100% |
| ssAAV2.v149-CB6-PI-Fluc | 4.14E12 | 67% |
| ssAAV2.v152-CB6-PI-Fluc | 7.44E12 | 120% |
| ssAAV2.MC1-CB6-PI-Fluc | 9.29E12 | 150% |

### Example 2. AAV2.MC1 Capsid Tropism and Transduction

It was examined whether the AAV2.MC1 capsid has unique tropisms to specific cell types within the mouse retina cell layer. Mouse retinas transduced via IVT injections using recombinant vectors encoding Egfp packaged with AAV2, AAV2.7m8, AAV2.v149, AAV2.v152, or AAV2.MC1 capsids were subjected to cryosectioning and immunofluorescence staining to investigate differential transduction between cell types conferred by the capsids. AAV2.MC1 was able to confer two-fold higher transduction in ganglion cells compared to AAV2 or AAV2.7m8 (FIG. 4). Additionally, there was increased transduction in amacrine, bipolar, and horizontal cells. In all non-photoreceptor cell types investigated, AAV2.MC1 showed the best performance.

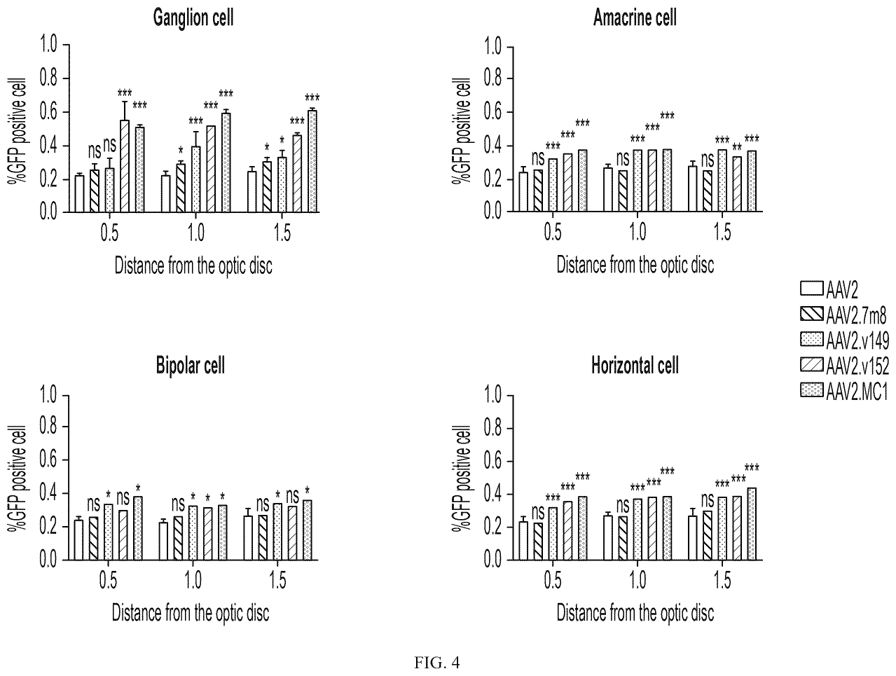

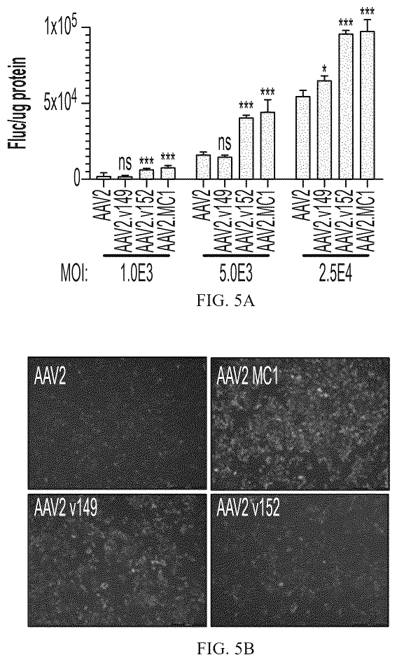

rAAVs produced using AAV2, AAV2.v149, AAV2.v152, and AAV2.MC1 capsid proteins were then used to transduce HeLa cells with ssAAV-Fluc vectors, and the results showed that the vectors packaged with AAV2.v149, AAV2.v152, and AAV2.MC1 capsids showed better transduction than the same vectors packaged with AAV2 (FIGS. 5A-5B).

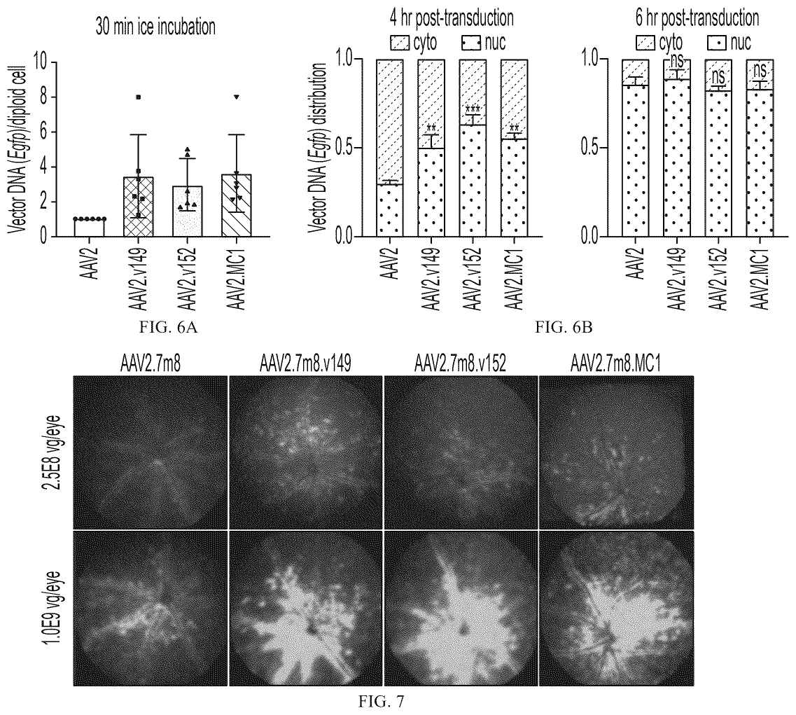

To explore whether the capsid protein variants effect post-entry mechanisms, the functions of the capsids were tested by cellular trafficking assays. HeLa cells were transduced with rAAV-Egfp vectors packaged with AAV2, AAV2.v149, AAV2.v152, or AAV2.MC1 capsids and allowed to sit on ice for 30 minutes. After 30 minutes, the cells were washed of uninternalized and unbound vector and harvested for DNA. Detection of vector DNA by ddPCR showed that vectors packaged with AAV2.v149, AAV2.v152, or AAV2.MC1 capsids showed higher internalization of vector than those packaged with AAV2 (FIG. 6A), indicating that the G36, N80, and A125 residues confer higher cell-surface binding than the wild-type AAV2 residues at those positions.

Vector-transduced cells were then harvested at four or six hours post-transduction, and cytoplasmic and nuclear fractions were separated to quantify vector genome DNA abundances in the two fractions to evaluate the relative locations of AAV particles during the initial phases of transduction. Compared to vectors packaged with AAV2, those packaged with AAV2.MC1 showed a higher distribution of nuclear-localized vector at four hours post-transduction (FIG. 6B, left). All vectors showed similar nuclear localization ratios at six hours post-transduction (FIG. 6B, right). Thus, AAV2.MC1 showed higher cell-surface binding and faster nuclear localization of vector compared to AAV2.

### Example 3. VP1u MC1 Chimeric Grafting

The VP1u regions from AAV2.v149, AAV2.v152, and AAV2.MC1 were grafted onto the AAV2.7m8 VP1u to determine whether they could likewise boost the relative performances of other capsids as “chimeric grafts”. When delivered by IVT at 2.5E8 or 1E9 vg/eye, vectors packaged with the chimeric capsids (AAV2.7m8.v149, AAV2.7m8.v152, and AAV2.7m8.MC1) had better transduction efficiencies in the mouse retina than the vector packaged with AAV2.7m8 (FIG. 7), indicating that MCi VP1u can be grafted onto other capsid proteins to boost transduction efficiency.

The VP1u region from AAV2.MC1 was also grafted onto the wild-type AAV8, wild-type AAV9, wild-type AAV1, wild-type AAV3b, and wild-type AAV5 capsid proteins to produce an AAV8 MCi capsid protein (SEQ ID NO: 14), an AAV9 MCi capsid protein (SEQ ID NO: 15), an AAV1 MCi capsid protein (SEQ ID NO: 16), an AAV3b MCi capsid protein (SEQ ID NO: 17), and an AAV5 MCi capsid proteins (SEQ ID NO: 18).

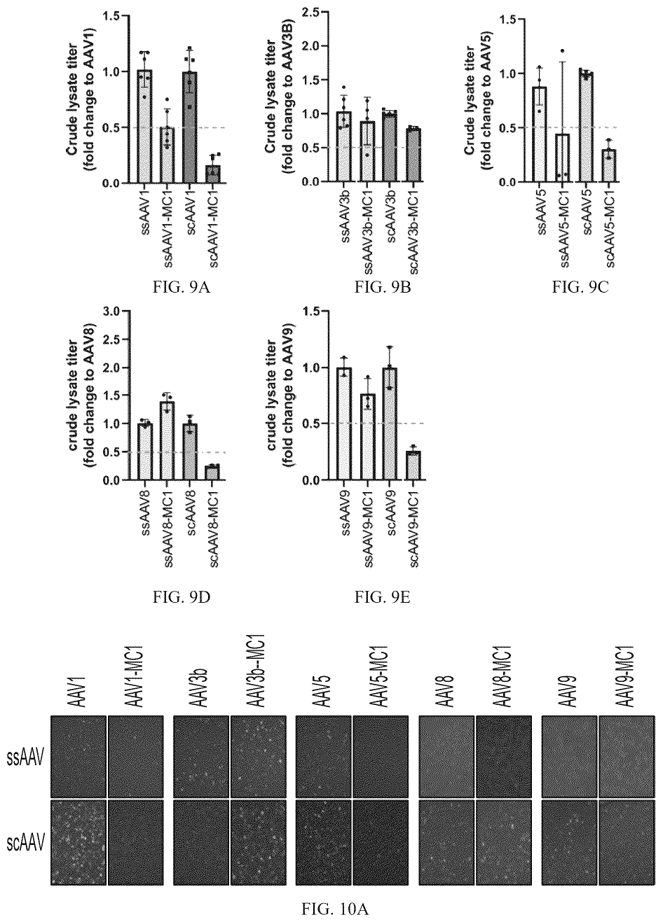

Each of the grafted MC1 capsid proteins were packaged with single-strand (ss) AAV or self-complementary (sc) AAV vectors. Packaging yields were assessed by crude lysate titration analyses with ddPCR (FIG. 9A-9E). The MCi graft resulted in >50% decrease in packaging yields of both ssAAV and scAAV for AAV1 MC1 and AAV5 MC1; >50% decrease in packaging yields of scAAV for AAV8 MC1 and AAV9 MC1; and no effect on packaging yields for AAV3b MCi.

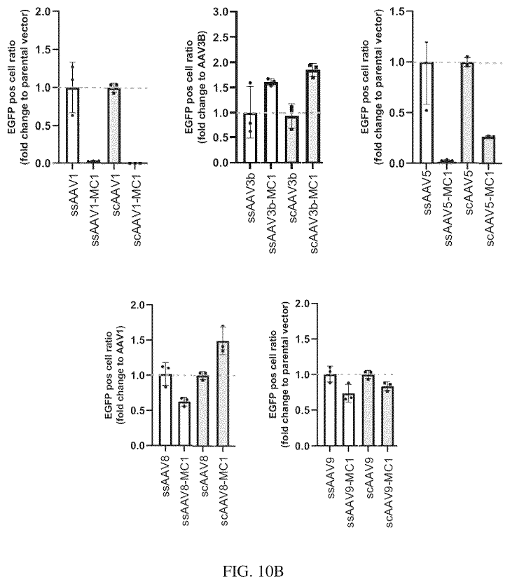

Each of the grafted MCi capsid proteins were packaged with ssAAV-CB6-Egfp or scAAVCB6-Egfp constructs by triple plasmid transfection in HEK293 cells. Wild-type AAV capsid proteins were used as control experiments. Crude lysates were collected from the producer cells and used to transduce mammalian cells (HeLa cells) in vitro. Epifluorescence microscopy imaging was used to observe transduction (EGFP fluorescence) (FIG. 10A). Flow cytometry was used to count the number of cells transduced with vector (FIG. 10B). As demonstrated by the data, the MCi modification resulted in loss of transduction for AAV1 MCi and AAV5 MCi relative to their respective wild-type proteins. Conversely, transduction efficiency was increased for AAV3b MCi and AAV8 MCi relative to their respective wild-type proteins. AAV9 MCi provided transduction efficiency that was comparable to wild-type AAV9.

These data collectively demonstrate that non-AAV2 capsid proteins can be modified to incorporate the AAV2 MCi VP1u region (e.g., comprising a glycine (G) at position 36, an asparagine (N) at position 80, and an alanine (A) at position 125) and retain or improve upon the functionality of wild-type capsid proteins.

### Example 4. Characterization of an AAV8 MC1 Capsid Protein

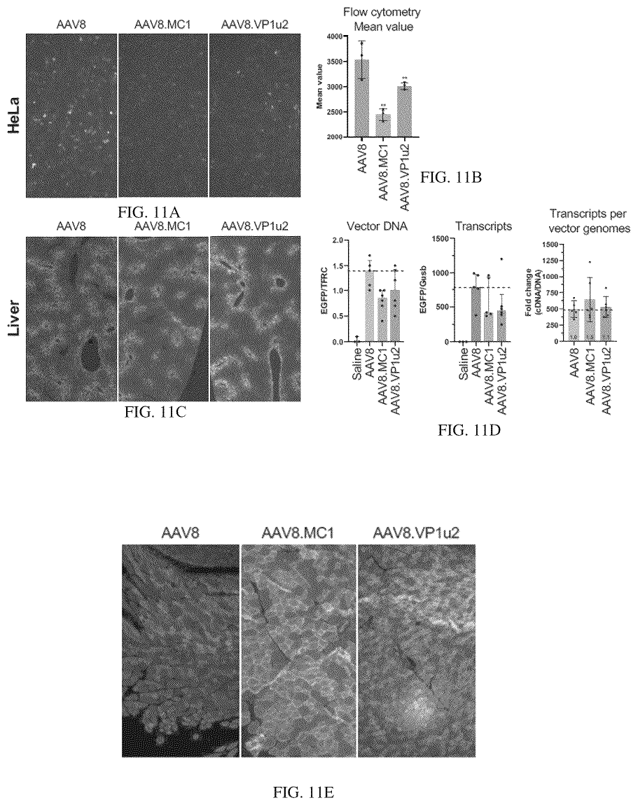

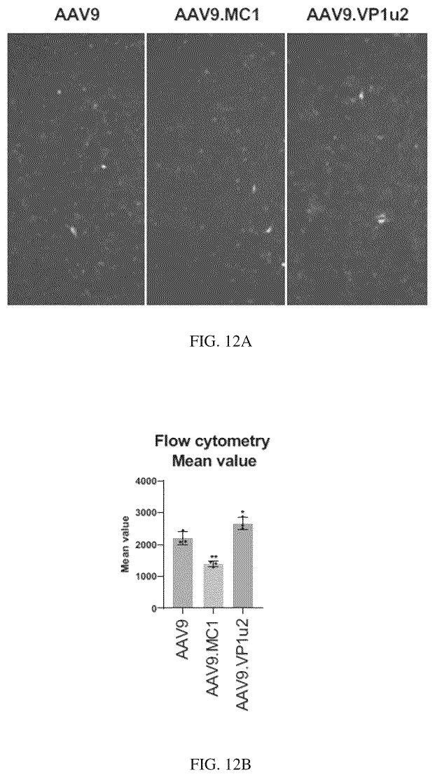

The AAV8 MCi capsid protein described in Example 3 was further characterized. First, rAAVs comprising the AAV8 MCi capsid protein (SEQ ID NO: 14) were tested for expression in vitro. Mammalian cells (HeLa cells) were transduced with AAV vectors comprising AAV8, AAV8-MC1, or the native VP1u sequence from prototypical AAV2 grafted onto AAV8 (AAV8-VP1u2) (5×10⁴ vector genomes/cell). 48 hours post-transduction, epifluorescence microscopy (FIG. 11A) and flow cytometry (FIG. 11B) were used to measure to qualify and quantify transduction. Second, rAAVs comprising the AAV8 MCi capsid protein were tested for expression in vivo. Adult C57BL/6 mice were injected with AAV vectors comprising AAV8, AAV8-MC1, or AAV8-VP1u2 by intravenous injections (tail vein). One month post-injection, mice were sacrificed and harvested for liver and muscle (quadriceps) tissues. Liver tissues were then subjected to cryosectioning and epifluorescence imaging (FIG. 11C), ddPCR (FIG. 11D), and muscle tissues were subjected to cryosectioning and epifluorescence imaging (FIG. 11E).

Grafting of either MCi or VP1u2 onto AAV8 reduced transduction in HeLa cells. The grafts did not significantly change transduction in mouse liver. Grafting of either MC1 or VP1u2 onto AAV8 increased transduction in mouse skeletal muscle.

### Example 5. Characterization of an AAV9 MC1 Capsid Protein

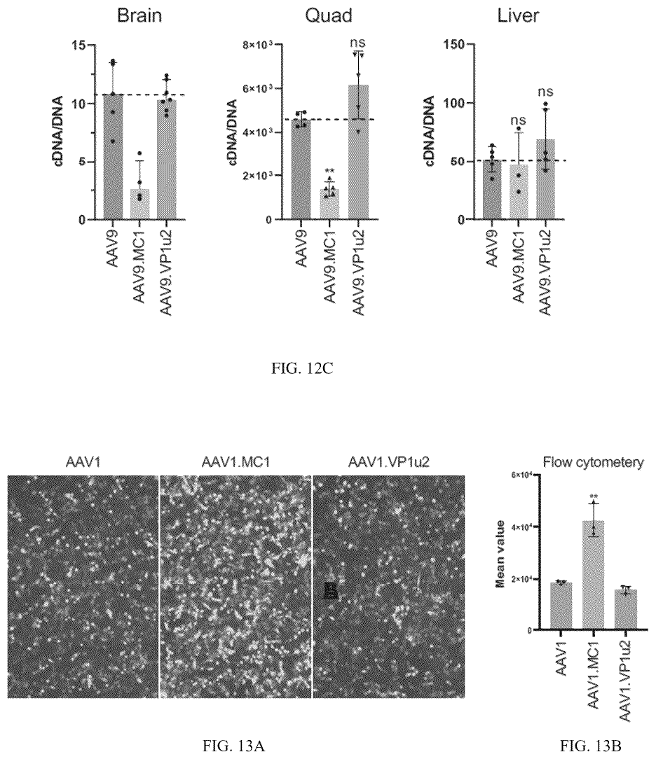

The AAV9 MCi capsid protein described in Example 3 was further characterized. First, rAAVs comprising the AAV9 MCi capsid protein (SEQ ID NO: 15) were tested for expression in vitro. Mammalian cells (HeLa cells) were transduced with AAV vectors comprising AAV9, AAV9-MC1, or AAV9-VP1u2 (5×10⁴ vector genomes). 48 hours post-transduction, epifluorescence microscopy (FIG. 12A) and flow cytometry (FIG. 12B) were used to measure to qualify and quantify transduction, respectively. Second, rAAVs comprising the AAV9 MCi capsid protein were tested for expression in vivo. Neonatal mice were injected with AAV vectors comprising AAV9, AAV9-MC1, or AAV9-VP1u2 (1.33×10¹¹ vg/mice). Four weeks post-injection, mice were sacrificed and harvested for brain, skeletal muscle (quadriceps), and liver (FIG. 12C).

Grafting MC1 onto AAV9 slightly reduced transduction in mammalian cells. There was no impact on transduction in the liver of mouse subjects. Grafting of VP1u2 onto AAV9 did not significantly alter transduction efficiency.

### Example 6. Characterization of an AAV1 MC1 Capsid Protein

The AAV1 MCi capsid protein described in Example 3 was further characterized. First, rAAVs comprising the AAV1 MCi (SEQ ID NO: 16), AAV1, or AAV1-VP1u2 capsid proteins were tested for expression in vitro. Mammalian cells (HeLa cells) were transduced with the rAAVs at 2.5×10⁴ vector genomes/cell. 48 hours post-transduction, cells were subjected to epifluorescence microscopy and flow cytometry (FIG. 13) to measure transduction.

Grafting of MC1 onto AAV1 increased transduction in mammalian cells.

### Example 7. Delivery of a Transgene to a Primate Using an AAV2 MC1 Capsid Protein

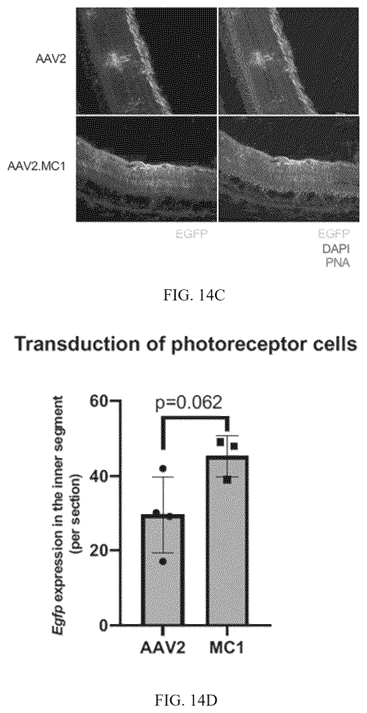

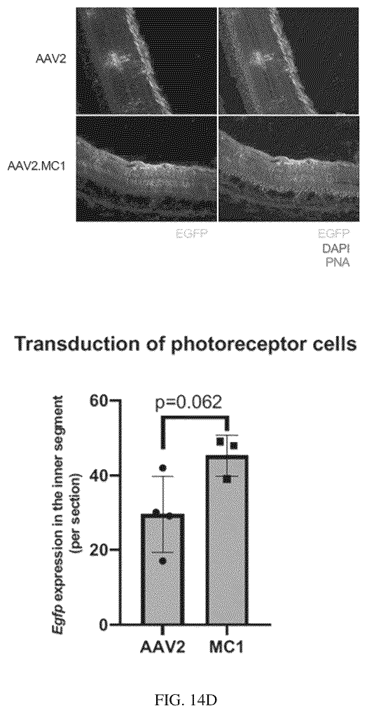

The ability of the AAV2-MC1 capsid protein comprising the amino acid sequence of SEQ ID NO: 4 to transduce ocular cells in non-human primates (NHPs) was investigated. A cynomolgus macaque was dosed with test vectors (5×10¹¹ vector genomes/eye) via intravitreal injections. One eye received scAAV2-CB6-EGFP and the other eye received scAAV2-MC1-CB6-EGFP. At day 0 (before injection) and after 30 days post-injection, the animal was imaged by fundus autofluorescence (FIG. 14A). Arrows indicate fluorescence signal from the macula. The animal was sacrificed and the eyes were harvested. Vector genomes and EGFP transcripts of the transduced eyes were quantified by ddPCR (FIG. 14B). The number of transcripts per vector genomes was also calculated to determine the contribution of the MCi modification towards AAV2 transduction. Quantifications were performed with technical duplicates, and the data averaged. A portion of the eyes were fixed and subjected to cryosectioning and immunofluorescence (IF) microscopy (FIG. 14C). Sections were stained for EGFP expression, DAPI, and PNA. Quantification of transduced photoreceptors was determined (FIG. 14D) (n=3-4 sections per eye).

ddPCR quantification suggests that AAV2-MC1 achieved weaker transduction of the retina than by AAV2. AAV2-MC1 showed a deeper penetrance of the retina, especially at the inner and outer nuclear layers, while AAV2 showed a stronger and more localized macular expression than achieved by AAV2-MC1. Importantly, the AAV2-MC1 capsid protein delivered a higher level of EGFP expression in the retina (as shown in FIG. 14D).

SELECTED SEQUENCES
Wild-type AAV2 capsid protein amino acid sequence
(SEQ ID NO: 1)
MAADGYLPDWLEDTLSEGIRQWWKLKPGPPPPKPAERHKDDSRGLVLPGYKYLGPFNGLDKGEP
VNEADAAALEHDKAYDRQLDSGDNPYLKYNHADAEFQERLKEDTSFGGNLGRAVFQAKKRVLEP
LGLVEEPVKTAPGKKRPVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVITTSTRTWALPTYNNHL
YKQISSQSGASNDNHYFGYSTPWGYFDFNRFHCHFSPRDWQRLINNNWGFRPKRLNFKLFNIQV
KEVTQNDGITTIANNLTSTVQVFTDSEYQLPYVLGSAHQGCLPPFPADVFMVPQYGYLTLNNGS
QAVGRSSFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKISADNNNSEYSWTGATKYHLNG
RDSLVNPGPAMASHKDDEEKFFPQSGVLIFGKQGSEKTNVDIEKVMITDEEEIRTTNPVATEQY
GSVSTNLQRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNY
NKSVNVDFTVDINGVYSEPRPIGTRYLTRNL
AAV2 v149 amino acid sequence-E36G, V125A
(SEQ ID NO: 2)
MAADGYLPDWLEDTLSEGIRQWWKLKPGPPPPKPAGRHKDDSRGLVLPGYKYLGPFNGLDKGEP
VNEADAAALEHDKAYDRQLDSGDNPYLKYNHADAEFQERLKEDTSFGGNLGRAVFQAKKRALEP
LGLVEEPVKTAPGKKRPVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVITTSTRTWALPTYNNHL
YKQISSQSGASNDNHYFGYSTPWGYFDFNRFHCHFSPRDWQRLINNNWGFRPKRLNFKLFNIQV
KEVTQNDGITTIANNLTSTVQVFTDSEYQLPYVLGSAHQGCLPPFPADVFMVPQYGYLTLNNGS
QAVGRSSFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQY
GSVSTNLQRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNY
NKSVNVDFTVDINGVYSEPRPIGTRYLTRNL
AAV2 v152 amino acid sequence-D80N
(SEQ ID NO: 3)
MAADGYLPDWLEDTLSEGIRQWWKLKPGPPPPKPAERHKDDSRGLVLPGYKYLGPENGLDKGEP
VNEADAAALEHDKAYNRQLDSGDNPYLKYNHADAEFQERLKEDTSFGGNLGRAVFQAKKRVLEP
LGLVEEPVKTAPGKKRPVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVITTSTRTWALPTYNNHL
YKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQY
GSVSTNLQRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNY
NKSVNVDFTVDINGVYSEPRPIGTRYLTRNL
AAV2 MC1 amino acid sequence-E36G, D80N, V125A
(SEQ ID NO: 4)
MAADGYLPDWLEDTLSEGIRQWWKLKPGPPPPKPAGRHKDDSRGLVLPGYKYLGPENGLDKGEP
VNEADAAALEHDKAYNRQLDSGDNPYLKYNHADAEFQERLKEDTSFGGNLGRAVFQAKKRALEP
LGLVEEPVKTAPGKKRPVEHSPVEPDSSSGTGKAGQQPARKRLNFGQTGDADSVPDPQPLGQPP
AAPSGLGTNTMATGSGAPMADNNEGADGVGNSSGNWHCDSTWMGDRVITTSTRTWALPTYNNHL
YKQISSQSGASNDNHYFGYSTPWGYFDFNRFHCHFSPRDWQRLINNNWGFRPKRLNFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFTFSYTFEDVPFHSSYAHSQSLDRLMNPLIDQYLYYLSRT
NTPSGTTTQSRLQFSQAGASDIRDQSRNWLPGPCYRQQRVSKTSADNNNSEYSWTGATKYHLNG
RDSLVNPGPAMASHKDDEEKFFPQSGVLIFGKQGSEKTNVDIEKVMITDEEEIRTTNPVATEQY
GSVSTNLQRGNRQAATADVNTQGVLPGMVWQDRDVYLQGPIWAKIPHTDGHFHPSPLMGGFGLK
HPPPQILIKNTPVPANPSTTFSAAKFASFITQYSTGQVSVEIEWELQKENSKRWNPEIQYTSNY
NKSVNVDFTVDINGVYSEPRPIGTRYLTRNL
AAV2 v149 nucleic acid sequence
(SEQ ID NO: 5)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGGGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACGACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGCTCTTGAACCT
CTGGGCCTGGTTGAGGAACCTGTTAAGACGGCTCCGGGAAAAAAGAGGCCGGTAGAGCACTCTC
CTGTGGAGCCAGACTCCTCCTCGGGAACCGGAAAGGCGGGCCAGCAGCCTGCAAGAAAAAGATT
GAATTTTGGTCAGACTGGAGACGCAGACTCAGTACCTGACCCCCAGCCTCTCGGACAGCCACCA
GCAGCCCCCTCTGGTCTGGGAACTAATACGATGGCTACAGGCAGTGGCGCACCAATGGCAGACA
ATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTC
TACAAACAAATTTCCAGCCAATCAGGAGCCTCGAACGACAATCACTACTTTGGCTACAGCACCC
CTTGGGGGTATTTTGACTTCAACAGATTCCACTGCCACTTTTCACCACGTGACTGGCAAAGACT
CATCAACAACAACTGGGGATTCCGACCCAAGAGACTCAACTTCAAGCTCTTTAACATTCAAGTC
AAAGAGGTCACGCAGAATGACGGTACGACGACGATTGCCAATAACCTTACCAGCACGGTTCAGG
TGTTTACTGACTCGGAGTACCAGCTCCCGTACGTCCTCGGCTCGGCGCATCAAGGATGCCTCCC
GCCGTTCCCAGCAGACGTCTTCATGGTGCCACAGTATGGATACCTCACCCTGAACAACGGGAGT
CAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAG
CCAGAGTCTGGACCGTCTCATGAATCCTCTCATCGACCAGTACCTGTATTACTTGAGCAGAACA
AACACTCCAAGTGGAACCACCACGCAGTCAAGGCTTCAGTTTTCTCAGGCCGGAGCGAGTGACA
TTCGGGACCAGTCTAGGAACTGGCTTCCTGGACCCTGTTACCGCCAGCAGCGAGTATCAAAGAC
ATCTGCGGATAACAACAACAGTGAATACTCGTGGACTGGAGCTACCAAGTACCACCTCAATGGC
AGAGACTCTCTGGTGAATCCGGGCCCGGCCATGGCAAGCCACAAGGACGATGAAGAAAAGTTTT
TTCCTCAGAGCGGGGTTCTCATCTTTGGGAAGCAAGGCTCAGAGAAAACAAATGTGGACATTGA
AAAGGTCATGATTACAGACGAAGAGGAAATCAGGACAACCAATCCCGTGGCTACGGAGCAGTAT
GGTTCTGTATCTACCAACCTCCAGAGAGGCAACAGACAAGCAGCTACCGCAGATGTCAACACAC
AAGGCGTTCTTCCAGGCATGGTCTGGCAGGACAGAGATGTGTACCTTCAGGGGCCCATCTGGGC
AAAGATTCCACACACGGACGGACATTTTCACCCCTCTCCCCTCATGGGTGGATTCGGACTTAAA
CACCCTCCTCCACAGATTCTCATCAAGAACACCCCGGTACCTGCGAATCCTTCGACCACCTTCA
GTGCGGCAAAGTTTGCTTCCTTCATCACACAGTACTCCACGGGACAGGTCAGCGTGGAGATCGA
GTGGGAGCTGCAGAAGGAAAACAGCAAACGCTGGAATCCCGAAATTCAGTACACTTCCAACTAC
AACAAGTCTGTTAATGTGGACTTTACTGTGGACACTAATGGCGTGTATTCAGAGCCTCGCCCCA
TTGGCACCAGATACCTGACTCGTAATCTGTAA
AAV2 v152 nucleic acid sequence
(SEQ ID NO: 6)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGAGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACAACCGGCAACTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGTTCTTGAACCT
CTGGGCCTGGTTGAGGAACCTGTTAAGACGGCTCCGGGAAAAAAGAGGCCGGTAGAGCACTCTC
CTGTGGAGCCAGACTCCTCCTCGGGAACCGGAAAGGCGGGCCAGCAGCCTGCAAGAAAAAGATT
GAATTTTGGTCAGACTGGAGACGCAGACTCAGTACCTGACCCCCAGCCTCTCGGACAGCCACCA
GCAGCCCCCTCTGGTCTGGGAACTAATACGATGGCTACAGGCAGTGGCGCACCAATGGCAGACA
ATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTC
TACAAACAAATTTCCAGCCAATCAGGAGCCTCGAACGACAATCACTACTTTGGCTACAGCACCC
CTTGGGGGTATTTTGACTTCAACAGATTCCACTGCCACTTTTCACCACGTGACTGGCAAAGACT
CATCAACAACAACTGGGGATTCCGACCCAAGAGACTCAACTTCAAGCTCTTTAACATTCAAGTC
AAAGAGGTCACGCAGAATGACGGTACGACGACGATTGCCAATAACCTTACCAGCACGGTTCAGG
TGTTTACTGACTCGGAGTACCAGCTCCCGTACGTCCTCGGCTCGGCGCATCAAGGATGCCTCCC
GCCGTTCCCAGCAGACGTCTTCATGGTGCCACAGTATGGATACCTCACCCTGAACAACGGGAGT
CAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAG
CCAGAGTCTGGACCGTCTCATGAATCCTCTCATCGACCAGTACCTGTATTACTTGAGCAGAACA
AACACTCCAAGTGGAACCACCACGCAGTCAAGGCTTCAGTTTTCTCAGGCCGGAGCGAGTGACA
TTCGGGACCAGTCTAGGAACTGGCTTCCTGGACCCTGTTACCGCCAGCAGCGAGTATCAAAGAC
ATCTGCGGATAACAACAACAGTGAATACTCGTGGACTGGAGCTACCAAGTACCACCTCAATGGC
AGGGACTCTCTGGTGAATCCGGGCCCGGCCATGGCAAGCCACAAGGACGATGAAGAAAAGTTTT
TTCCTCAGAGCGGGGTTCTCATCTTTGGGAAGCAAGGCTCAGAGAAAACAAATGTGGACATTGA
AAAGGTCATGATTACAGACGAAGAGGAAATCAGGACAACCAATCCCGTGGCTACGGAGCAGTAT
GGTTCTGTATCTACCAACCTCCAGAGAGGCAACAGACAAGCAGCTACCGCAGATGTCAACACAC
AAGGCGTTCTTCCAGGCATGGTCTGGCAGGACAGAGATGTGTACCTTCAGGGGCCCATCTGGGC
AAAGATTCCACACACGGACGGACATTTTCACCCCTCTCCCCTCATGGGTGGATTCGGACTTAAA
CACCCTCCTCCACAGATTCTCATCAAGAACACCCCGGTACCTGCGAATCCTTCGACCACCTTCA
GTGCGGCAAAGTTTGCTTCCTTCATCACACAGTACTCCACGGGACAGGTCAGCGTGGAGATCGA
GTGGGAGCTGCAGAAGGAAAACAGCAAACGCTGGAATCCCGAAATTCAGTACACTTCCAACTAC
AACAAGTCTGTTAATGTGGACTTTACTGTGGACACTAATGGCGTGTATTCAGAGCCTCGCCCCA
TTGGCACCAGATACCTGACTCGTAATCTGTAA
AAV2 MC1 nucleic acid sequence
(SEQ ID NO: 7)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGGGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACAACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGCTCTTGAACCT
CTGGGCCTGGTTGAGGAACCTGTTAAGACGGCTCCGGGAAAAAAGAGGCCGGTAGAGCACTCTC
CTGTGGAGCCAGACTCCTCCTCGGGAACCGGAAAGGCGGGCCAGCAGCCTGCAAGAAAAAGATT
GAATTTTGGTCAGACTGGAGACGCAGACTCAGTACCTGACCCCCAGCCTCTCGGACAGCCACCA
GCAGCCCCCTCTGGTCTGGGAACTAATACGATGGCTACAGGCAGTGGCGCACCAATGGCAGACA
ATAACGAGGGCGCCGACGGAGTGGGTAATTCCTCGGGAAATTGGCATTGCGATTCCACATGGAT
GGGCGACAGAGTCATCACCACCAGCACCCGAACCTGGGCCCTGCCCACCTACAACAACCACCTC
TACAAACAAATTTCCAGCCAATCAGGAGCCTCGAACGACAATCACTACTTTGGCTACAGCACCC
CTTGGGGGTATTTTGACTTCAACAGATTCCACTGCCACTTTTCACCACGTGACTGGCAAAGACT
CATCAACAACAACTGGGGATTCCGACCCAAGAGACTCAACTTCAAGCTCTTTAACATTCAAGTC
AAAGAGGTCACGCAGAATGACGGTACGACGACGATTGCCAATAACCTTACCAGCACGGTTCAGG
TGTTTACTGACTCGGAGTACCAGCTCCCGTACGTCCTCGGCTCGGCGCATCAAGGATGCCTCCC
GCCGTTCCCAGCAGACGTCTTCATGGTGCCACAGTATGGATACCTCACCCTGAACAACGGGAGT
CAGGCAGTAGGACGCTCTTCATTTTACTGCCTGGAGTACTTTCCTTCTCAGATGCTGCGTACCG
GAAACAACTTTACCTTCAGCTACACTTTTGAGGACGTTCCTTTCCACAGCAGCTACGCTCACAG
CCAGAGTCTGGACCGTCTCATGAATCCTCTCATCGACCAGTACCTGTATTACTTGAGCAGAACA
AACACTCCAAGTGGAACCACCACGCAGTCAAGGCTTCAGTTTTCTCAGGCCGGAGCGAGTGACA
TTCGGGACCAGTCTAGGAACTGGCTTCCTGGACCCTGTTACCGCCAGCAGCGAGTATCAAAGAC
ATCTGCGGATAACAACAACAGTGAATACTCGTGGACTGGAGCTACCAAGTACCACCTCAATGGC
AGAGACTCTCTGGTGAATCCGGGCCCGGCCATGGCAAGCCACAAGGACGATGAAGAAAAGTTTT
TTCCTCAGAGCGGGGTTCTCATCTTTGGGAAGCAAGGCTCAGAGAAAACAAATGTGGACATTGA
AAAGGTCATGATTACAGACGAAGAGGAAATCAGGACAACCAATCCCGTGGCTACGGAGCAGTAT
GGTTCTGTATCTACCAACCTCCAGAGAGGCAACAGACAAGCAGCTACCGCAGATGTCAACACAC
AAGGCGTTCTTCCAGGCATGGTCTGGCAGGACAGAGATGTGTACCTTCAGGGGCCCATCTGGGC
AAAGATTCCACACACGGACGGACATTTTCACCCCTCTCCCCTCATGGGTGGATTCGGACTTAAA
CACCCTCCTCCACAGATTCTCATCAAGAACACCCCGGTACCTGCGAATCCTTCGACCACCTTCA
GTGCGGCAAAGTTTGCTTCCTTCATCACACAGTACTCCACGGGACAGGTCAGCGTGGAGATCGA
GTGGGAGCTGCAGAAGGAAAACAGCAAACGCTGGAATCCCGAAATTCAGTACACTTCCAACTAC
AACAAGTCTGTTAATGTGGACTTTACTGTGGACACTAATGGCGTGTATTCAGAGCCTCGCCCCA
TTGGCACCAGATACCTGACTCGTAATCTGTAA
Wild-type AAV8 capsid protein amino acid sequence
(SEQ ID NO: 9)
MAADGYLPDWLEDNLSEGIREWWALKPGAPKPKANQQKQDDGRGLVLPGYKYLGPFNGLDKGEP
VNAADAAALEHDKAYDQQLQAGDNPYLRYNHADAEFQERLQEDTSFGGNLGRAVFQAKKRVLEP
LGLVEEGAKTAPGKKRPVEPSPQRSPDSSTGIGKKGQQPARKRLNFGQTGDSESVPDPQPLGEP
PAAPSGVGPNTMAAGGGAPMADNNEGADGVGSSSGNWHCDSTWLGDRVITTSTRTWALPTYNNH
LYKQISNGTSGGATNDNTYFGYSTPWGYFDFNRFHCHFSPRDWQRLINNNWGFRPKRLSFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFQFTYTFEDVPFHSSYAHSQSLDRLMNPLIDQYLYYL
SRTQTTGGTANTQTLGFSQGGPNTMANQAKNWLPGPCYRQQRVSTTTGQNNNSNFAWTAGTKYH
LNGRNSLANPGIAMATHKDDEERFFPSNGILIFGKQNAARDNADYSDVMLTSEEEIKTTNPVAT
EEYGIVADNLQQQNTAPQIGTVNSQGALPGMVWQNRDVYLQGPIWAKIPHTDGNFHPSPLMGGF
GLKHPPPQILIKNTPVPADPPTTFNQSKLNSFITQYSTGQVSVEIEWELQKENSKRWNPEIQYT
SNYYKSTSVDFAVNTEGVYSEPRPIGTRYLTRNL
Wild-type AAV9 capsid protein amino acid sequence
(SEQ ID NO: 10)
MAADGYLPDWLEDNLSEGIREWWALKPGAPQPKANQQHQDNARGLVLPGYKYLGPGNGLDKGEP
VNAADAAALEHDKAYDQQLKAGDNPYLKYNHADAEFQERLKEDTSFGGNLGRAVFQAKKRLLEP
LGLVEEAAKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
KTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQAQAQTGWVQNQGILPGMVWQDRDVYLQGPIWAKIPHTDGNFHPSPLMGGFGM
KHPPPQILIKNTPVPADPPTAFNKDKLNSFITQYSTGQVSVEIEWELQKENSKRWNPEIQYTSN
YYKSNNVEFAVNTEGVYSEPRPIGTRYLTRNL
Wild-type AAV1 capsid protein amino acid sequence
(SEQ ID NO: 11)
MAADGYLPDWLEDNLSEGIREWWDLKPGAPKPKANQQKQDDGRGLVLPGYKYLGPFNGLDKGEP
VNAADAAALEHDKAYDQQLKAGDNPYLRYNHADAEFQERLQEDTSFGGNLGRAVFQAKKRVLEP
LGLVEEGAKTAPGKKRPVEQSPQEPDSSSGIGKTGQQPAKKRLNFGQTGDSESVPDPQPLGEPP
ATPAAVGPTTMASGGGAPMADNNEGADGVGNASGNWHCDSTWLGDRVITTSTRTWALPTYNNHL
YKQISSASTGASNDNHYFGYSTPWGYFDFNRFHCHFSPRDWQRLINNNWGFRPKRLNFKLFNIQ
VKEVTTNDGVTTIANNLTSTVQVFSDSEYQLPYVLGSAHQGCLPPFPADVFMIPQYGYLTLNNG
SQAVGRSSFYCLEYFPSQMLRTGNNFTFSYTFEEVPFHSSYAHSQSLDRLMNPLIDQYLYYLNR
TQNQSGSAQNKDLLFSRGSPAGMSVQPKNWLPGPCYRQQRVSKTKTDNNNSNFTWTGASKYNLN
GRESIINPGTAMASHKDDEDKFFPMSGVMIFGKESAGASNTALDNVMITDEEEIKATNPVATER
FGTVAVNFQSSSTDPATGDVHAMGALPGMVWQDRDVYLQGPIWAKIPHTDGHFHPSPLMGGFGL
KNPPPQILIKNTPVPANPPAEFSATKFASFITQYSTGQVSVEIEWELQKENSKRWNPEVQYTSN
YAKSANVDFTVDNNGLYTEPRPIGTRYLTRPL
Wild-type AAV3B capsid protein amino acid sequence
(SEQ ID NO: 12)
MAADGYLPDWLEDNLSEGIREWWALKPGVPQPKANQQHQDNRRGLVLPGYKYLGPGNGLDKGEP
VNEADAAALEHDKAYDQQLKAGDNPYLKYNHADAEFQERLQEDTSFGGNLGRAVFQAKKRILEP
LGLVEEAAKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDTNGVYSEPRPIGTRYLTRNL*
Wild-type AAV5 capsid protein amino acid sequence
(SEQ ID NO: 13)
MSFVDHPPDWLEEVGEGLREFLGLEAGPPKPKPNQQHQDQARGLVLPGYNYLGPGNGLDRGEPV
NRADEVAREHDISYNEQLEAGDNPYLKYNHADAEFQEKLADDTSFGGNLGKAVFQAKKRVLEPF
GLVEEGAKTAPTGKRIDDHFPKRKKARTEEDSKPSTSSDAEAGPSGSQQLQIPAQPASSLGADT
MSAGGGGPLGDNNQGADGVGNASGDWHCDSTWMGDRVVTKSTRTWVLPSYNNHQYREIKSGSVD
GSNANAYFGYSTPWGYFDFNRFHSHWSPRDWQRLINNYWGFRPRSLRVKIFNIQVKEVTVQDST
TTIANNLTSTVQVFTDDDYQLPYVVGNGTEGCLPAFPPQVFTLPQYGYATLNRDNTENPTERSS
FFCLEYFPSKMLRTGNNFEFTYNFEEVPFHSSFAPSQNLFKLANPLVDQYLYRFVSTNNTGGVQ
FNKNLAGRYANTYKNWFPGPMGRTQGWNLGSGVNRASVSAFATTNRMELEGASYQVPPQPNGMT
NNLQGSNTYALENTMIFNSQPANPGTTATYLEGNMLITSESETQPVNRVAYNVGGQMATNNQSS
TTAPATGTYNLQEIVPGSVWMERDVYLQGPIWAKIPETGAHFHPSPAMGGFGLKHPPPMMLIKN
TPVPGNITSFSDVPVSSFITQYSTGQVTVEMEWELKKENSKRWNPEIQYTNNYNDPQFVDFAPD
STGEYRTTRPIGTRYLTRPL
AAV8 MC1 capsid protein amino acid sequence
(SEQ ID: 14)
MAADGYLPDWLEDTLSEGIRQWWKLKPGPPPPKPAGRHKDDSRGLVLPGYKYLGPFNGLDKGEP
VNEADAAALEHDKAYNRQLDSGDNPYLKYNHADAEFQERLKEDTSFGGNLGRAVFQAKKRALEP
LGLVEEPVKTAPGKKRPVEPSPQRSPDSSTGIGKKGQQPARKRLNFGQTGDSESVPDPQPLGEP
PAAPSGVGPNTMAAGGGAPMADNNEGADGVGSSSGNWHCDSTWLGDRVITTSTRTWALPTYNNH
LYKQISNGTSGGATNDNTYFGYSTPWGYFDFNRFHCHFSPRDWQRLINNNWGFRPKRLSFKLFN
IQVKEVTQNEGTKTIANNLTSTIQVFTDSEYQLPYVLGSAHQGCLPPFPADVFMIPQYGYLTLN
NGSQAVGRSSFYCLEYFPSQMLRTGNNFQFTYTFEDVPFHSSYAHSQSLDRLMNPLIDQYLYYL
SRTQTTGGTANTQTLGFSQGGPNTMANQAKNWLPGPCYRQQRVSTTTGQNNNSNFAWTAGTKYH
LNGRNSLANPGIAMATHKDDEERFFPSNGILIFGKQNAARDNADYSDVMLTSEEEIKTTNPVAT
EEYGIVADNLQQQNTAPQIGTVNSQGALPGMVWQNRDVYLQGPIWAKIPHTDGNFHPSPLMGGF
GLKHPPPQILIKNTPVPADPPTTFNQSKLNSFITQYSTGQVSVEIEWELQKENSKRWNPEIQYT
SNYYKSTSVDFAVNTEGVYSEPRPIGTRYLTRNL
AAV9 MC1 capsid protein amino acid sequence
(SEQ ID: 15)
MAADGYLPDWLEDTLSEGIRQWWKLKPGPPPPKPAGRHKDDSRGLVLPGYKYLGPFNGLDKGEP
VNEADAAALEHDKAYNRQLDSGDNPYLKYNHADAEFQERLKEDTSFGGNLGRAVFQAKKRALEP
LGLVEEPVKTAPGKKRPVEQSPQEPDSSAGIGKSGAQPAKKRLNFGQTGDTESVPDPQPIGEPP
AAPSGVGSLTMASGGGAPVADNNEGADGVGSSSGNWHCDSQWLGDRVITTSTRTWALPTYNNHL
YKQISNSTSGGSSNDNAYFGYSTPWGYFDFNRFHCHFSPRDWQRLINNNWGFRPKRLNFKLFNI
QVKEVTDNNGVKTIANNLTSTVQVFTDSDYQLPYVLGSAHEGCLPPFPADVFMIPQYGYLTLND
GSQAVGRSSFYCLEYFPSQMLRTGNNFQFSYEFENVPFHSSYAHSQSLDRLMNPLIDQYLYYLS
KTINGSGQNQQTLKFSVAGPSNMAVQGRNYIPGPSYRQQRVSTTVTQNNNSEFAWPGASSWALN
GRNSLMNPGPAMASHKEGEDRFFPLSGSLIFGKQGTGRDNVDADKVMITNEEEIKTTNPVATES
YGQVATNHQSAQAQAQTGWVQNQGILPGMVWQDRDVYLQGPIWAKIPHTDGNFHPSPLMGGFGM
KHPPPQILIKNTPVPADPPTAFNKDKLNSFITQYSTGQVSVEIEWELQKENSKRWNPEIQYTSN
YYKSNNVEFAVNTEGVYSEPRPIGTRYLTRNL
AAV1 MC1 capsid protein amino acid sequence
(SEQ ID: 16)
MAADGYLPDWLEDTLSEGIRQWWKLKPGPPPPKPAGRHKDDSRGLVLPGYKYLGPFNGLDKGEP
VNEADAAALEHDKAYNRQLDSGDNPYLKYNHADAEFQERLKEDTSFGGNLGRAVFQAKKRALEP
LGLVEEPVKTAPGKKRPVEQSPQEPDSSSGIGKTGQQPAKKRLNFGQTGDSESVPDPQPLGEPP
ATPAAVGPTTMASGGGAPMADNNEGADGVGNASGNWHCDSTWLGDRVITTSTRTWALPTYNNHL
YKQISSASTGASNDNHYFGYSTPWGYFDFNRFHCHFSPRDWQRLINNNWGFRPKRLNFKLFNIQ
VKEVTTNDGVTTIANNLTSTVQVFSDSEYQLPYVLGSAHQGCLPPFPADVFMIPQYGYLTLNNG
SQAVGRSSFYCLEYFPSQMLRTGNNFTFSYTFEEVPFHSSYAHSQSLDRLMNPLIDQYLYYLNR
TQNQSGSAQNKDLLFSRGSPAGMSVQPKNWLPGPCYRQQRVSKTKTDNNNSNFTWTGASKYNLN
GRESIINPGTAMASHKDDEDKFFPMSGVMIFGKESAGASNTALDNVMITDEEEIKATNPVATER
FGTVAVNFQSSSTDPATGDVHAMGALPGMVWQDRDVYLQGPIWAKIPHTDGHFHPSPLMGGFGL
KNPPPQILIKNTPVPANPPAEFSATKFASFITQYSTGQVSVEIEWELQKENSKRWNPEVQYTSN
YAKSANVDFTVDNNGLYTEPRPIGTRYLTRPL
AAV3B MC1 capsid protein amino acid sequence
(SEQ ID: 17)
MAADGYLPDWLEDTLSEGIRQWWKLKPGPPPPKPAGRHKDDSRGLVLPGYKYLGPFNGLDKGEP
VNEADAAALEHDKAYNRQLDSGDNPYLKYNHADAEFQERLKEDTSFGGNLGRAVFQAKKRALEP
LGLVEEPVKTAPGKKRPVDQSPQEPDSSSGVGKSGKQPARKRLNFGQTGDSESVPDPQPLGEPP
AAPTSLGSNTMASGGGAPMADNNEGADGVGNSSGNWHCDSQWLGDRVITTSTRTWALPTYNNHL
YKQISSQSGASNDNHYFGYSTPWGYFDFNRFHCHFSPRDWQRLINNNWGFRPKKLSFKLFNIQV
KEVTQNDGTTTIANNLTSTVQVFTDSEYQLPYVLGSAHQGCLPPFPADVFMVPQYGYLTLNNGS
QAVGRSSFYCLEYFPSQMLRTGNNFQFSYTFEDVPFHSSYAHSQSLDRLMNPLIDQYLYYLNRT
QGTTSGTTNQSRLLFSQAGPQSMSLQARNWLPGPCYRQQRLSKTANDNNNSNFPWTAASKYHLN
GRDSLVNPGPAMASHKDDEEKFFPMHGNLIFGKEGTTASNAELDNVMITDEEEIRTTNPVATEQ
YGTVANNLQSSNTAPTTRTVNDQGALPGMVWQDRDVYLQGPIWAKIPHTDGHFHPSPLMGGFGL
KHPPPQIMIKNTPVPANPPTTFSPAKFASFITQYSTGQVSVEIEWELQKENSKRWNPEIQYTSN
YNKSVNVDFTVDINGVYSEPRPIGTRYLTRNL*
AAV5 MC1 capsid protein amino acid sequence
(SEQ ID: 18)
MAADGYLPDWLEDTLSEGIRQWWKLKPGPPPPKPAGRHKDDSRGLVLPGYKYLGPFNGLDKGEP
VNEADAAALEHDKAYNRQLDSGDNPYLKYNHADAEFQERLKEDTSFGGNLGRAVFQAKKRALEP
LGLVEEPVKAPTGKRIDDHFPKRKKARTEEDSKPSTSSDAEAGPSGSQQLQIPAQPASSLGADT
MSAGGGGPLGDNNQGADGVGNASGDWHCDSTWMGDRVVTKSTRTWVLPSYNNHQYREIKSGSVD
GSNANAYFGYSTPWGYFDFNRFHSHWSPRDWQRLINNYWGFRPRSLRVKIFNIQVKEVTVQDST
TTIANNLTSTVQVFTDDDYQLPYVVGNGTEGCLPAFPPQVFTLPQYGYATLNRDNTENPTERSS
FFCLEYFPSKMLRTGNNFEFTYNFEEVPFHSSFAPSQNLFKLANPLVDQYLYRFVSTNNTGGVQ
FNKNLAGRYANTYKNWFPGPMGRTQGWNLGSGVNRASVSAFATTNRMELEGASYQVPPQPNGMT
NNLQGSNTYALENTMIFNSQPANPGTTATYLEGNMLITSESETQPVNRVAYNVGGQMATNNQSS
TTAPATGTYNLQEIVPGSVWMERDVYLQGPIWAKIPETGAHFHPSPAMGGFGLKHPPPMMLIKN
TPVPGNITSFSDVPVSSFITQYSTGQVTVEMEWELKKENSKRWNPEIQYTNNYNDPQFVDFAPD
STGEYRTTRPIGTRYLTRPL
AAV8 MC1 capsid protein nucleic acid sequence
(SEQ ID: 19)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGGGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACAACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGCTCTTGAACCT
CTGGGCCTGGTTGAGGAACCTGTTAAGacggctcctggaaagaagagaccggtagagccatcac
cccagcgttctccagactcctctacgggcatcggcaagaaaggccaacagcccgccagaaaaag
actcaattttggtcagactggcgactcagagtcagttccagaccctcaacctctcggagaacct
ccagcagcgccctctggtgtgggacctaatacaatggctgcaggcggtggcgcaccaatggcag
acaataacgaaggcgccgacggagtgggtagttcctcgggaaattggcattgcgattccacatg
gctgggcgacagagtcatcaccaccagcacccgaacctgggccctgcccacctacaacaaccac
ctctacaagcaaatctccaacgggacatcgggaggagccaccaacgacaacacctacttcggct
acagcaccccctgggggtattttgactttaacagattccactgccacttttcaccacgtgactg
gcagcgactcatcaacaacaactggggattccggcccaagagactcagcttcaagctcttcaac
atccaggtcaaggaggtcacgcagaatgaaggcaccaagaccatcgccaataacctcaccagca
ccatccaggtgtttacggactcggagtaccagctgccgtacgttctcggctctgcccaccaggg
ctgcctgcctccgttcccggcggacgtgttcatgattccccagtacggctacctaacactcaac
aacggtagtcaggccgtgggacgctcctccttctactgcctggaatactttccttcgcagatgc
tgagaaccggcaacaacttccagtttacttacaccttcgaggacgtgcctttccacagcagcta
cgcccacagccagagcttggaccggctgatgaatcctctgattgaccagtacctgtactacttg
tctcggactcaaacaacaggaggcacggcaaatacgcagactctgggcttcagccaaggtgggc
ctaatacaatggccaatcaggcaaagaactggctgccaggaccctgttaccgccaacaacgcgt
ctcaacgacaaccgggcaaaacaacaatagcaactttgcctggactgctgggaccaaataccat
ctgaatggaagaaattcattggctaatcctggcatcgctatggcaacacacaaagacgacgagg
agcgtttttttcccagtaacgggatcctgatttttggcaaacaaaatgctgccagagacaatgc
ggattacagcgatgtcatgctcaccagcgaggaagaaatcaaaaccactaaccctgtggctaca
gaggaatacggtatcgtggcagataacttgcagcagcaaaacacggctcctcaaattggaactg
tcaacagccagggggccttacccggtatggtctggcagaaccgggacgtgtacctgcagggtcc
catctgggccaagattcctcacacggacggcaacttccacccgtctccgctgatgggcggcttt
ggcctgaaacatcctccgcctcagatcctgatcaagaacacgcctgtacctgcggatcctccga
ccaccttcaaccagtcaaagctgaactctttcatcacgcaatacagcaccggacaggtcagcgt
ggaaattgaatgggagctgcagaaggaaaacagcaagcgctggaaccccgagatccagtacacc
tccaactactacaaatctacaagtgtggactttgctgttaatacagaaggcgtgtactctgaac
cccgccccattggcacccgttacctcacccgtaatctg
AAV9 MC1 capsid protein nucleic acid sequence
(SEQ ID: 20)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGGGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACAACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGCTCTTGAACCT
CTGGGCCTGGTTGAGGAACCTGTTAAGacggctcctggaaagaagaggcctgtagagcagtctc
ctcaggaaccggactcctccgcgggtattggcaaatcgggtgcacagcccgctaaaaagagact
caatttcggtcagactggcgacacagagtcagtcccagaccctcaaccaatcggagaacctccc
gcagccccctcaggtgtgggatctcttacaatggcttcaggtggtggcgcaccagtggcagaca
ataacgaaggtgccgatggagtgggtagttcctcgggaaattggcattgcgattcccaatggct
gggggacagagtcatcaccaccagcacccgaacctgggccctgcccacctacaacaatcacctc
tacaagcaaatctccaacagcacatctggaggatcttcaaatgacaacgcctacttcggctaca
gcaccccctgggggtattttgacttcaacagattccactgccacttctcaccacgtgactggca
gcgactcatcaacaacaactggggattccggcctaagcgactcaacttcaagctcttcaacatt
caggtcaaagaggttacggacaacaatggagtcaagaccatcgccaataaccttaccagcacgg
tccaggtcttcacggactcagactatcagctcccgtacgtgctcgggtcggctcacgagggctg
cctcccgccgttcccagcggacgttttcatgattcctcagtacgggtatctgacgcttaatgat
ggaagccaggccgtgggtcgttcgtccttttactgcctggaatatttcccgtcgcaaatgctaa
gaacgggtaacaacttccagttcagctacgagtttgagaacgtacctttccatagcagctacgc
tcacagccaaagcctggaccgactaatgaatccactcatcgaccaatacttgtactatctctca
aagactattaacggttctggacagaatcaacaaacgctaaaattcagtgtggccggacccagca
acatggctgtccagggaagaaactacatacctggacccagctaccgacaacaacgtgtctcaac
cactgtgactcaaaacaacaacagcgaatttgcttggcctggagcttcttcttgggctctcaat
ggacgtaatagcttgatgaatcctggacctgctatggccagccacaaagaaggagaggaccgtt
tctttcctttgtctggatctttaatttttggcaaacaaggaactggaagagacaacgtggatgc
ggacaaagtcatgataaccaacgaagaagaaattaaaactactaacccggtagcaacggagtcc
tatggacaagtggccacaaaccaccagagtgcccaagcacaggcgcagaccggctgggttcaaa
accaaggaatacttccgggtatggtttggcaggacagagatgtgtacctgcaaggacccatttg
ggccaaaattcctcacacggacggcaactttcacccttctccgctgatgggagggtttggaatg
aagcacccgcctcctcagatcctcatcaaaaacacacctgtacctgggatcctccaacggcctt
caacaaggacaagctgaactctttcatcacccagtattctactggccaagtcagcgtggagatc
gagtgggagctgcagaaggaaaacagcaagcgctggaacccggagatccagtacacttccaact
attacaagtctaataatgttgaatttgctgttaatactgaaggtgtatatagtgaaccccgccc
cattggcaccagatacctgactcgtaatctg
AAV1 MC1 capsid protein nucleic acid sequence
(SEQ ID: 21)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGGGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACAACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGCTCTTGAACCT
CTGGGCCTGGTTGAGGAACCTGTTAAGacggctcctggaaagaaacgtccggtagagcagtcgc
cacaagagccagactcctcctcgggcatcggcaagacaggccagcagcccgctaaaaagagact
caattttggtcagactggcgactcagagtcagtccccgatccacaacctctggagaacctccag
caacccccgctgctgtgggacctactacaatggcttcaggggggcgcaccaatggcagacaata
acgaaggcgccgacggagtgggtaatgcctcaggaaattggcattgcgattccacatggctggg
cgacagagtcatcaccaccagcacccgcacctgggccttgcccacctacaataaccacctctac
aagcaaatctccagtgcttcaacgggggccagcaacgacaaccactacttcggctacagcaccc
cctgggggtattttgatttcaacagattccactgccacttttcaccacgtgactggcagcgact
catcaacaacaattggggattccggcccaagagactcaacttcaaactcttcaacatccaagtc
aaggaggtcacgacgaatgatggcgtcacaaccatcgctaataaccttaccagcacggttcaag
tcttctcggactcggagtaccagcttccgtacgtcctcggctctgcgcaccagggctgcctccc
tccgttcccggcggacgtgttcatgattccgcaatacggctacctgacgctcaacaatggcagc
caagccgtgggacgttcatccttttactgcctggaatatttcccttctcagatgctgagaacgg
gcaacaactttaccttcagctacacctttgaggaagtgcctttccacagcagctacgcgcacag
ccagagcctggaccggctgatgaatcctctcatcgaccaatacctgtattacctgaacagaact
caaaatcagtccggaagtgcccaaaacaaggacttgctgtttagccgtgggtctccagctggca
tgtctgttcagcccaaaaactggctacctggaccctgttatcggcagcagcgcgtttctaaaac
aaaaacagacaacaacaacagcaattttacctggactggtgcttcaaaatataacctcaatggg
cgtgaatccatcatcaaccctggcactgctatggcctcacacaaagacgacgaagacaagttct
ttcccatgagcggtgtcatgatttttggaaaagagagcgccggagcttcaaacactgcattgga
caatgtcatgattacagacgaagaggaaattaaagccactaaccctgtggccaccgaaagattt
gggaccgtggcagtcaatttccagagcagcagcacagaccctgcgaccggagatgtgcatgcta
tgggagcattacctggcatggtgtggcaagatagagacgtgtacctgcagggtcccatttgggc
caaaattcctcacacagatggacactttcacccgtctcctcttatgggggctttggactcaaga
acccgcctcctcagatcctcatcaaaaacacgcctgttcctgcgaatcctccggcggagttttc
agctacaaagtttgcttcattcatcacccaatactccacaggacaagtgagtgtggaaattgaa
tgggagctgcagaaagaaaacagcaagcgctggaatcccgaagtgcagtacacatccaattatg
caaaatctgccaacgttgattttactgtggacaacaatggactttatactgagcctcgccccat
tggcacccgttaccttacccgtcccctg
AAV3B MC1 capsid protein nucleic acid sequence
(SEQ ID: 22)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGGGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACAACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGCTCTTGAACCT
CTGGGCCTGGTTGAGGAACCTGTTAAGacggctcctggaaagaagaggcctgtagatcagtctc
ctcaggaaccggactcatcatctggtgttggcaaatcgggcaaacagcctgccagaaaaagact
aaatttcggtcagactggcgactcagagtcagtcccagaccctcaacctctcggagaaccacca
gcagcccccacaagtttgggatctaatacaatggcttcaggcggtggcgcaccaatggcagaca
ataacgagggtgccgatggagtgggtaattcctcaggaaattggcattgcgattcccaatggct
gggcgacagagtcatcaccaccagcaccagaacctgggccctgcccacttacaacaaccatctc
tacaagcaaatctccagccaatcaggagcttcaaacgacaaccactactttggctacagcaccc
cttgggggtattttgactttaacagattccactgccacttctcaccacgtgactggcagcgact
cattaacaacaactggggattccggcccaagaaactcagcttcaagctcttcaacatccaagtt
aaagaggtcacgcagaacgatggcacgacgactattgccaataaccttaccagcacggttcaag
tgtttacggactcggagtatcagctcccgtacgtgctcgggtcggcgcaccaaggctgtctccc
gccgtttccagcggacgtcttcatggtccctcagtatggatacctcaccctgaacaacggaagt
caagcggtgggacgctcatccttttactgcctggagtacttcccttcgcagatgctaaggactg
gaaataacttccaattcagctataccttcgaggatgtaccttttcacagcagctacgctcacag
ccagagtttggatcgcttgatgaatcctcttattgatcagtatctgtactacctgaacagaacg
caaggaacaacctctggaacaaccaaccaatcacggctgctttttagccaggctgggcctcagt
ctatgtctttgcaggccagaaattggctacctgggccctgctaccggcaacagagactttcaaa
gactgctaacgacaacaacaacagtaactttccttggacagcggccagcaaatatcatctcaat
ggccgcgactcgctggtgaatccaggaccagctatggccagtcacaaggacgatgaagaaaaat
ttttccctatgcacggcaatctaatatttggcaaagaagggacaacggcaagtaacgcagaatt
agataatgtaatgattacggatgaagaagagattcgtaccaccaatcctgtggcaacagagcag
tatggaactgtggcaaataacttgcagagctcaaatacagctcccacgactagaactgtcaatg
atcagggggccttacctggcatggtgtggcaagatcgtgacgtgtaccttcaaggacctatctg
ggcaaagattcctcacacggatggacactttcatccttctcctctgatgggaggctttggactg
aaacatccgcctcctcaaatcatgatcaaaaatactccggtaccggcaaatcctccgacgactt
tcagcccggccaagtttgcttcatttatcactcagtactccactggacaggtcagcgtggaaat
tgagtgggagctacagaaagaaaacagcaaacgttggaatccagagattcagtacacttccaac
tacaacaagtctgttaatgtggactttactgtagacactaatggtgtttatagtgaacctcgcc
ctattggaacccggtatctcacacgaaacttgtaa
AAV5 MC1 capsid protein nucleic acid sequence
(SEQ ID: 23)
ATGGCTGCCGATGGTTATCTTCCAGATTGGCTCGAGGACACTCTCTCTGAAGGAATAAGACAGT
GGTGGAAGCTCAAACCTGGCCCACCACCACCAAAGCCCGCAGGGCGGCATAAGGACGACAGCAG
GGGTCTTGTGCTTCCTGGGTACAAGTACCTCGGACCCTTCAACGGACTCGACAAGGGAGAGCCG
GTCAACGAGGCAGACGCCGCGGCCCTCGAGCACGACAAAGCCTACAACCGGCAGCTCGACAGCG
GAGACAACCCGTACCTCAAGTACAACCACGCCGACGCGGAGTTTCAGGAGCGCCTTAAAGAAGA
TACGTCTTTTGGGGGCAACCTCGGACGAGCAGTCTTCCAGGCGAAAAAGAGGGCTCTTGAACCT
CTGGGCCTGGTTGAGGAACCTGTTAAGgcccctaccggaaagcggatagacgaccactttccaa
aaagaaagaaggcccggaccgaagaggactccaagccttccacctcgtcagacgccgaagctgg
acccagcggatcccagcagctgcaaatcccagcccaaccagcctcaagtttgggagctgataca
atgtctgcgggaggtggcggcccattgggcgacaataaccaaggtgccgatggagtgggcaatg
cctcgggagattggcattgcgattccacgtggatgggggacagagtcgtcaccaagtccacccg
aacctgggtgctgcccagctacaacaaccaccagtaccgagagatcaaaagcggctccgtcgac
ggaagcaacgccaacgcctactttggatacagcaccccctgggggtactttgactttaaccgct
tccacagccactggagcccccgagactggcaaagactcatcaacaactactggggcttcagacc
ccggtccctcagagtcaaaatcttcaacattcaagtcaaagaggtcacggtgcaggactccacc
accaccatcgccaacaacctcacctccaccgtccaagtgtttacggacgacgactaccagctgc
cctacgtcgtcggcaacgggaccgagggatgcctgccggccttccctccgcaggtctttacgct
gccgcagtacggttacgcgacgctgaaccgcgacaacacagaaaatcccaccgagaggagcagc
ttcttctgcctagagtactttcccagcaagatgctgagaacgggcaacaactttgagtttacct
acaactttgaggaggtgcccttccactccagcttcgctcccagtcagaacctcttcaagctggc
caacccgctggtggaccagtacttgtaccgcttcgtgagcacaaataacactggcggagtccag
ttcaacaagaacctggccgggagatacgccaacacctacaaaaactggttcccggggcccatgg
gccgaacccagggctggaacctgggctccggggtcaaccgcgccagtgtcagcgccttcgccac
gaccaataggatggagctcgagggcgcgagttaccaggtgcccccgcagccgaacggcatgacc
aacaacctccagggcagcaacacctatgccctggagaacactatgatcttcaacagccagccgg
cgaacccgggcaccaccgccacgtacctcgagggcaacatgctcatcaccagcgagagcgagac
gcagccggtgaaccgcgtggcgtacaacgtcggcgggcagatggccaccaacaaccagagctcc
accactgcccccgcgaccggcacgtacaacctccaggaaatcgtgcccggcagcgtgtggatgg
agagggacgtgtacctccaaggacccatctgggccaagatcccagagacgggggcgcactttca
cccctctccggccatgggcggattcggactcaaacacccaccgcccatgatgctcatcaagaac
acgcctgtgcccggaaatatcaccagcttctcggacgtgcccgtcagcagcttcatcacccagt
acagcaccgggcaggtcaccgtggagatggagtgggagctcaagaaggaaaactccaagaggtg
gaacccagagatccagtacacaaacaactacaacgacccccagtttgtggactttgccccggac
agcaccggggaatacagaaccaccagacctatcggaacccgataccttacccgacccctt

### EQUIVALENTS

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

The terms “about” and “substantially” preceding a numerical value represent ±10% of the recited numerical value.

1. An isolated capsid protein comprising at least three amino acid mutations within a VP1 unique (VP1u) region of the capsid protein, wherein the at least three amino acid mutations are in positions corresponding to positions 36, 80, and 125 with reference to amino acid position numbering of a wild-type AAV2 capsid protein, optionally wherein the wild-type AAV2 capsid protein comprises the amino acid sequence of SEQ ID NO: 1.

2. The isolated capsid protein of claim 1, wherein the capsid protein is of a serotype selected from AAV1, AAV2, AAV3, AAV4, AAV5, AAV6, AAV7, AAV8, AAV9, AAV10, AAV9.PHPeB, MYOAAV, AAVrh74, AAV2.5T, AAV2/8, AAV2/6, AAVrh32.33, Anc80, NP40, NP59, and LKO3.

3. The isolated capsid protein of claim 1, wherein the VP1u region comprises an amino acid sequence that is at least 70%, at least 80%, at least 90%, or at least 95% identical to the amino acid sequence set forth in SEQ ID NO: 1.

4. The isolated capsid protein of claim 1, wherein the at least three amino acid substitutions comprise E36G, D80N, and V125A amino acid substitutions with reference to amino acid position numbering of a wild-type AAV2 capsid protein.

5. The isolated capsid protein of claim 1, wherein the capsid protein comprises a glycine (G) at position 36, an asparagine (N) at position 80, and an alanine (A) at position 125 with reference to amino acid position numbering of a wild-type AAV2 capsid protein.

6. The isolated capsid protein of claim 1, wherein the capsid protein comprises the amino acid sequence of any one of SEQ ID NOs: 4 or 14-18.

7. A recombinant adeno-associated virus (rAAV) comprising:
(i) an isolated nucleic acid comprising a transgene encoding a gene product flanked by AAV ITRs; and
(ii) the isolated capsid protein of claim 1.

8. The rAAV of claim 7, wherein the gene product is a protein or interfering nucleic acid.

9. The rAAV of claim 8, wherein the protein is a therapeutic protein or the interfering nucleic acid is selected from a dsRNA, siRNA, miRNA, artificial miRNA (ami-RNA), or RNA aptamer.

10. The rAAV of claim 7, wherein the transgene further comprises a promoter, optionally wherein the promoter is a constitutive promoter, inducible promoter, or a tissue-specific promoter.

11. The rAAV of claim 7, wherein the transgene further comprises one or more miRNA binding sites.

12. A method for delivering a transgene to a target cell of a subject, the method comprising administering to the subject the rAAV of claim 7,
wherein the target cell is an ocular cell, central nervous system (CNS) cell, peripheral nervous system (PNS) cell, liver cell, lung cell, muscle cell, bone cell, pancreas cell, stomach cell, or skin cell, and/or
wherein the subject is a mammal, optionally wherein the subject is a human.

13.-14. (canceled)

15. The method of claim 12, wherein the administration comprises injection or topical administration.

16. The method of claim 12, wherein the administration results in increased expression of the transgene in the target cell relative to expression of the transgene delivered using an rAAV comprising a wild-type AAV capsid protein.

17. A method for delivering a transgene to an ocular cell in a subject, the method comprising administering to the subject a recombinant adeno-associated virus (rAAV) comprising:
(i) an isolated nucleic acid comprising a transgene encoding one or more gene products flanked by AAV ITRs; and
(ii) an adeno-associated virus (AAV) capsid protein comprising the amino acid sequence set forth in SEQ ID NO: 4.

18. The method of claim 17, wherein the administration comprises intraocular administration, intravenous administration, or topical administration to the eye or eyelid,
wherein the intraocular administration comprises intravitreal administration, transscleral administration, subconjunctival administration, retrobulbar administration, intracameral administration, or subretinal administration,
wherein the ocular cell is an amacrine cell, a bipolar cell, a trabecular meshwork cell, a ciliary body cell, a retinal pigment epithelial cell, a retinal cell, an astrocyte, a pericyte, a Müller cell, a ganglion cell, or a photoreceptor cell, and/or
wherein the subject is a mammal, optionally wherein the mammal is a human, and/or
wherein the transgene encoding one or more gene products further comprises a promoter, optionally an eye-specific promoter, further optionally wherein the eye-specific promoter is a retinoschisin proximal promoter, interphotoreceptor retinoid-binding protein enhancer (RS/IRBPa) promoter, rhodopsin kinase (RK) promoter, RPE65 promoter, or human cone opsin promoter.

19.-22. (canceled)

23. The method of claim 17, wherein the one or more gene products comprise a protein or an inhibitory nucleic acid.

24. The method of claim 23, wherein the protein comprises an anti-VEGF agent, optionally wherein the anti-VEGF agent is KH902.

25. An isolated AAV capsid protein comprising an amino acid sequence having the sequence as set forth in any one of SEQ ID NOs: 4 or 14-18.

26. A recombinant AAV (rAAV) comprising the isolated AAV capsid protein of claim 25.


SEQUENCE LISTING (34 sequences)

SEQ ID NO: 1 - protein, 735 aa, unidentified [sequence omitted]

SEQ ID NO: 2 - protein, 735 aa, synthetic construct
MAADGYLPDWLEDTLSEGIRQWWKLKPGPPPPKPAGRHKDDSRGLVLPGYKYLGPFNGLDKGEPVNEADAAALEHDKAYDRQLDSGDNPYLKYNHADAEFQERLKEDTSFGGNLGRAVFQAKKRALEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 3 - protein, 735 aa, synthetic construct
MAADGYLPDWLEDTLSEGIRQWWKLKPGPPPPKPAERHKDDSRGLVLPGYKYLGPFNGLDKGEPVNEADAAALEHDKAYNRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 4 - protein, 735 aa, synthetic construct
MAADGYLPDWLEDTLSEGIRQWWKLKPGPPPPKPAGRHKDDSRGLVLPGYKYLGPFNGLDKGEPVNEADAAALEHDKAYNRQLDSGDNPYLKYNHADAEFQERLKEDTSFGGNLGRAVFQAKKRALEPLGLVEEPVKTAPGKKRPVEHSPVEPDSSSGTGKAGQQPARKRLNFGQTGDADSVPDPQPLGQPPAAPSGLGTNTMATGSGAPMADNNEGADGVGNSSGNWHCDSTWMGDRVITTSTRTWALPTYNNHLYKQISSQSGASNDNHYFGYSTPWGYFDFNRFHCHFSPRDWQRLINNNWGFRPKRLNFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFTFSYTFEDVPFHSSYAHSQSLDRLMNPLIDQYLYYLSRTNTPSGTTTQSRLQFSQAGASDIRDQSRNWLPGPCYRQQRVSKTSADNNNSEYSWTGATKYHLNGRDSLVNPGPAMASHKDDEEKFFPQSGVLIFGKQGSEKTNVDIEKVMITDEEEIRTTNPVATEQYGSVSTNLQRGNRQAATADVNTQGVLPGMVWQDRDVYLQGPIWAKIPHTDGHFHPSPLMGGFGLKHPPPQILIKNTPVPANPSTTFSAAKFASFITQYSTGQVSVEIEWELQKENSKRWNPEIQYTSNYNKSVNVDFTVDTNGVYSEPRPIGTRYLTRNL

SEQ ID NO: 5 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagggcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgcggagtttcaggagcgccttaaagaagatacgtcttttgggggcaacctcggacgagcagtcttccaggcgaaaaagagggctcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 6 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacaaccggcaactcgacagcggagacaacccgtacctcaagtacaaccacgccgacgcggagtttcaggagcgccttaaagaagatacgtcttttgggggcaacctcggacgagcagtcttccaggcgaaaaagagggttcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagggactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 7 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagggcggcataaggacgacagcaggggtcttgtgcttcctgggtacaagtacctcggacccttcaacggactcgacaagggagagccggtcaacgaggcagacgccgcggccctcgagcacgacaaagcctacaaccggcagctcgacagcggagacaacccgtacctcaagtacaaccacgccgacgcggagtttcaggagcgccttaaagaagatacgtcttttgggggcaacctcggacgagcagtcttccaggcgaaaaagagggctcttgaacctctgggcctggttgaggaacctgttaagacggctccgggaaaaaagaggccggtagagcactctcctgtggagccagactcctcctcgggaaccggaaaggcgggccagcagcctgcaagaaaaagattgaattttggtcagactggagacgcagactcagtacctgacccccagcctctcggacagccaccagcagccccctctggtctgggaactaatacgatggctacaggcagtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaaacaaatttccagccaatcaggagcctcgaacgacaatcactactttggctacagcaccccttgggggtattttgacttcaacagattccactgccacttttcaccacgtgactggcaaagactcatcaacaacaactggggattccgacccaagagactcaacttcaagctctttaacattcaagtcaaagaggtcacgcagaatgacggtacgacgacgattgccaataaccttaccagcacggttcaggtgtttactgactcggagtaccagctcccgtacgtcctcggctcggcgcatcaaggatgcctcccgccgttcccagcagacgtcttcatggtgccacagtatggatacctcaccctgaacaacgggagtcaggcagtaggacgctcttcattttactgcctggagtactttccttctcagatgctgcgtaccggaaacaactttaccttcagctacacttttgaggacgttcctttccacagcagctacgctcacagccagagtctggaccgtctcatgaatcctctcatcgaccagtacctgtattacttgagcagaacaaacactccaagtggaaccaccacgcagtcaaggcttcagttttctcaggccggagcgagtgacattcgggaccagtctaggaactggcttcctggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctcctccacagattctcatcaagaacaccccggtacctgcgaatccttcgaccaccttcagtgcggcaaagtttgcttccttcatcacacagtactccacgggacaggtcagcgtggagatcgagtgggagctgcagaaggaaaacagcaaacgctggaatcccgaaattcagtacacttccaactacaacaagtctgttaatgtggactttactgtggacactaatggcgtgtattcagagcctcgccccattggcaccagatacctgactcgtaatctgtaa

SEQ ID NO: 8 - DNA, 1659 bp, synthetic construct
  source 1..1659: mol_type = other DNA
atggtcagctactgggacaccggggtcctgctgtgcgcgctgctcagctgtctgcttctcacaggatctagttccggaggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaaccttttgttgcttttggaagtggcatggaatctctggtggaagccacggtgggggagcgtgtcagaatccctgcgaagtaccttggttacccacccccagaaataaaatggtataaaaatggaataccccttgagtccaatcacacaattaaagcggggcatgtactgacgattatggaagtgagtgaaagagacacaggaaattacactgtcatccttaccaatcccatttcaaaggagaagcagagccatgtggtctctctggttgtgtatgtcccaccgggcccgggcgacaaaactcacacatgcccactgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctagtcaaaggcttctatcccagcgacatcgccgtggagtgggagagcaatgggcagccggagaacaactacaaggccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatga

SEQ ID NO: 9 - protein, 738 aa, unidentified
  source 1..738: note = Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 10 - protein, 736 aa, unidentified
  source 1..736: note = Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 11 - protein, 736 aa, unidentified
  source 1..736: note = Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 12 - protein, 736 aa, unidentified
  source 1..736: note = Adeno-associated virus 3B
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 13 - protein, 724 aa, unidentified
  source 1..724: note = Adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 14 - protein, 738 aa, synthetic construct
MAADGYLPDWLEDTLSEGIRQWWKLKPGPPPPKPAGRHKDDSRGLVLPGYKYLGPFNGLDKGEPVNEADAAALEHDKAYNRQLDSGDNPYLKYNHADAEFQERLKEDTSFGGNLGRAVFQAKKRALEPLGLVEEPVKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 15 - protein, 736 aa, synthetic construct
MAADGYLPDWLEDTLSEGIRQWWKLKPGPPPPKPAGRHKDDSRGLVLPGYKYLGPFNGLDKGEPVNEADAAALEHDKAYNRQLDSGDNPYLKYNHADAEFQERLKEDTSFGGNLGRAVFQAKKRALEPLGLVEEPVKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 16 - protein, 736 aa, synthetic construct
MAADGYLPDWLEDTLSEGIRQWWKLKPGPPPPKPAGRHKDDSRGLVLPGYKYLGPFNGLDKGEPVNEADAAALEHDKAYNRQLDSGDNPYLKYNHADAEFQERLKEDTSFGGNLGRAVFQAKKRALEPLGLVEEPVKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 17 - protein, 736 aa, synthetic construct
MAADGYLPDWLEDTLSEGIRQWWKLKPGPPPPKPAGRHKDDSRGLVLPGYKYLGPFNGLDKGEPVNEADAAALEHDKAYNRQLDSGDNPYLKYNHADAEFQERLKEDTSFGGNLGRAVFQAKKRALEPLGLVEEPVKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 18 - protein, 724 aa, synthetic construct
MAADGYLPDWLEDTLSEGIRQWWKLKPGPPPPKPAGRHKDDSRGLVLPGYKYLGPFNGLDKGEPVNEADAAALEHDKAYNRQLDSGDNPYLKYNHADAEFQERLKEDTSFGGNLGRAVFQAKKRALEPLGLVEEPVKAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 19 - DNA, 2214 bp, synthetic construct
  source 1..2214: mol_type = other DNA
atggctgccgatggttatcttccagattggctcgaggacactctctctgaaggaataagacagtggtggaagctcaaacctggcccaccaccaccaaagcccgcagggcggcataaggacgacagcaggggtcttgtgcttcctgggtacaagtacctcggacccttcaacggactcgacaagggagagccggtcaacgaggcagacgccgcggccctcgagcacgacaaagcctacaaccggcagctcgacagcggagacaacccgtacctcaagtacaaccacgccgacgcggagtttcaggagcgccttaaagaagatacgtcttttgggggcaacctcggacgagcagtcttccaggcgaaaaagagggctcttgaacctctgggcctggttgaggaacctgttaagacggctcctggaaagaagagaccggtagagccatcaccccagcgttctccagactcctctacgggcatcggcaagaaaggccaacagcccgccagaaaaagactcaattttggtcagactggcgactcagagtcagttccagaccctcaacctctcggagaacctccagcagcgccctctggtgtgggacctaatacaatggctgcaggcggtggcgcaccaatggcagacaataacgaaggcgccgacggagtgggtagttcctcgggaaattggcattgcgattccacatggctgggcgacagagtcatcaccaccagcacccgaacctgggccctgcccacctacaacaaccacctctacaagcaaatctccaacgggacatcgggaggagccaccaacgacaacacctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacggcaaatacgcagactctgggcttcagccaaggtgggcctaatacaatggccaatcaggcaaagaactggctgccaggaccctgttaccgccaacaacgcgtctcaacgacaaccgggcaaaacaacaatagcaactttgcctggactgctgggaccaaataccatctgaatggaagaaattcattggctaatcctggcatcgctatggcaacacacaaagacgacgaggagcgtttttttcccagtaacgggatcctgatttttggcaaacaaaatgctgccagagacaatgcggattacagcgatgtcatgctcaccagcgaggaagaaatcaaaaccactaaccctgtggctacagaggaatacggtatcgtggcagataacttgcagcagcaaaacacggctcctcaaattggaactgtcaacagccagggggccttacccggtatggtctggcagaaccgggacgtgtacctgcagggtcccatctgggccaagattcctcacacggacggcaacttccacccgtctccgctgatgggcggctttggcctgaaacatcctccgcctcagatcctgatcaagaacacgcctgtacctgcggatcctccgaccaccttcaaccagtcaaagctgaactctttcatcacgcaatacagcaccggacaggtcagcgtggaaattgaatgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctg

SEQ ID NO: 20 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagggcggcataaggacgacagcaggggtcttgtgcttcctgggtacaagtacctcggacccttcaacggactcgacaagggagagccggtcaacgaggcagacgccgcggccctcgagcacgacaaagcctacaaccggcagctcgacagcggagacaacccgtacctcaagtacaaccacgccgacgcggagtttcaggagcgccttaaagaagatacgtcttttgggggcaacctcggacgagcagtcttccaggcgaaaaagagggctcttgaacctctgggcctggttgaggaacctgttaagacggctcctggaaagaagaggcctgtagagcagtctcctcaggaaccggactcctccgcgggtattggcaaatcgggtgcacagcccgctaaaaagagactcaatttcggtcagactggcgacacagagtcagtcccagaccctcaaccaatcggagaacctcccgcagccccctcaggtgtgggatctcttacaatggcttcaggtggtggcgcaccagtggcagacaataacgaaggtgccgatggagtgggtagttcctcgggaaattggcattgcgattcccaatggctgggggacagagtcatcaccaccagcacccgaacctgggccctgcccacctacaacaatcacctctacaagcaaatctccaacagcacatctggaggatcttcaaatgacaacgcctacttcggctacagcaccccctgggggtattttgacttcaacagattccactgccacttctcaccacgtgactggcagcgactcatcaacaacaactggggattccggcctaagcgactcaacttcaagctcttcaacattcaggtcaaagaggttacggacaacaatggagtcaagaccatcgccaataaccttaccagcacggtccaggtcttcacggactcagactatcagctcccgtacgtgctcgggtcggctcacgagggctgcctcccgccgttcccagcggacgttttcatgattcctcagtacgggtatctgacgcttaatgatggaagccaggccgtgggtcgttcgtccttttactgcctggaatatttcccgtcgcaaatgctaagaacgggtaacaacttccagttcagctacgagtttgagaacgtacctttccatagcagctacgctcacagccaaagcctggaccgactaatgaatccactcatcgaccaatacttgtactatctctcaaagactattaacggttctggacagaatcaacaaacgctaaaattcagtgtggccggacccagcaacatggctgtccagggaagaaactacatacctggacccagctaccgacaacaacgtgtctcaaccactgtgactcaaaacaacaacagcgaatttgcttggcctggagcttcttcttgggctctcaatggacgtaatagcttgatgaatcctggacctgctatggccagccacaaagaaggagaggaccgtttctttcctttgtctggatctttaatttttggcaaacaaggaactggaagagacaacgtggatgcggacaaagtcatgataaccaacgaagaagaaattaaaactactaacccggtagcaacggagtcctatggacaagtggccacaaaccaccagagtgcccaagcacaggcgcagaccggctgggttcaaaaccaaggaatacttccgggtatggtttggcaggacagagatgtgtacctgcaaggacccatttgggccaaaattcctcacacggacggcaactttcacccttctccgctgatgggagggtttggaatgaagcacccgcctcctcagatcctcatcaaaaacacacctgtacctgcggatcctccaacggccttcaacaaggacaagctgaactctttcatcacccagtattctactggccaagtcagcgtggagatcgagtgggagctgcagaaggaaaacagcaagcgctggaacccggagatccagtacacttccaactattacaagtctaataatgttgaatttgctgttaatactgaaggtgtatatagtgaaccccgccccattggcaccagatacctgactcgtaatctg

SEQ ID NO: 21 - DNA, 2208 bp, synthetic construct
  source 1..2208: mol_type = other DNA
atggctgccgatggttatcttccagattggctcgaggacactctctctgaaggaataagacagtggtggaagctcaaacctggcccaccaccaccaaagcccgcagggcggcataaggacgacagcaggggtcttgtgcttcctgggtacaagtacctcggacccttcaacggactcgacaagggagagccggtcaacgaggcagacgccgcggccctcgagcacgacaaagcctacaaccggcagctcgacagcggagacaacccgtacctcaagtacaaccacgccgacgcggagtttcaggagcgccttaaagaagatacgtcttttgggggcaacctcggacgagcagtcttccaggcgaaaaagagggctcttgaacctctgggcctggttgaggaacctgttaagacggctcctggaaagaaacgtccggtagagcagtcgccacaagagccagactcctcctcgggcatcggcaagacaggccagcagcccgctaaaaagagactcaattttggtcagactggcgactcagagtcagtccccgatccacaacctctcggagaacctccagcaacccccgctgctgtgggacctactacaatggcttcaggcggtggcgcaccaatggcagacaataacgaaggcgccgacggagtgggtaatgcctcaggaaattggcattgcgattccacatggctgggcgacagagtcatcaccaccagcacccgcacctgggccttgcccacctacaataaccacctctacaagcaaatctccagtgcttcaacgggggccagcaacgacaaccactacttcggctacagcaccccctgggggtattttgatttcaacagattccactgccacttttcaccacgtgactggcagcgactcatcaacaacaattggggattccggcccaagagactcaacttcaaactcttcaacatccaagtcaaggaggtcacgacgaatgatggcgtcacaaccatcgctaataaccttaccagcacggttcaagtcttctcggactcggagtaccagcttccgtacgtcctcggctctgcgcaccagggctgcctccctccgttcccggcggacgtgttcatgattccgcaatacggctacctgacgctcaacaatggcagccaagccgtgggacgttcatccttttactgcctggaatatttcccttctcagatgctgagaacgggcaacaactttaccttcagctacacctttgaggaagtgcctttccacagcagctacgcgcacagccagagcctggaccggctgatgaatcctctcatcgaccaatacctgtattacctgaacagaactcaaaatcagtccggaagtgcccaaaacaaggacttgctgtttagccgtgggtctccagctggcatgtctgttcagcccaaaaactggctacctggaccctgttatcggcagcagcgcgtttctaaaacaaaaacagacaacaacaacagcaattttacctggactggtgcttcaaaatataacctcaatgggcgtgaatccatcatcaaccctggcactgctatggcctcacacaaagacgacgaagacaagttctttcccatgagcggtgtcatgatttttggaaaagagagcgccggagcttcaaacactgcattggacaatgtcatgattacagacgaagaggaaattaaagccactaaccctgtggccaccgaaagatttgggaccgtggcagtcaatttccagagcagcagcacagaccctgcgaccggagatgtgcatgctatgggagcattacctggcatggtgtggcaagatagagacgtgtacctgcagggtcccatttgggccaaaattcctcacacagatggacactttcacccgtctcctcttatgggcggctttggactcaagaacccgcctcctcagatcctcatcaaaaacacgcctgttcctgcgaatcctccggcggagttttcagctacaaagtttgcttcattcatcacccaatactccacaggacaagtgagtgtggaaattgaatgggagctgcagaaagaaaacagcaagcgctggaatcccgaagtgcagtacacatccaattatgcaaaatctgccaacgttgattttactgtggacaacaatggactttatactgagcctcgccccattggcacccgttaccttacccgtcccctg

SEQ ID NO: 22 - DNA, 2211 bp, synthetic construct
  source 1..2211: mol_type = other DNA
atggctgccgatggttatcttccagattggctcgaggacactctctctgaaggaataagacagtggtggaagctcaaacctggcccaccaccaccaaagcccgcagggcggcataaggacgacagcaggggtcttgtgcttcctgggtacaagtacctcggacccttcaacggactcgacaagggagagccggtcaacgaggcagacgccgcggccctcgagcacgacaaagcctacaaccggcagctcgacagcggagacaacccgtacctcaagtacaaccacgccgacgcggagtttcaggagcgccttaaagaagatacgtcttttgggggcaacctcggacgagcagtcttccaggcgaaaaagagggctcttgaacctctgggcctggttgaggaacctgttaagacggctcctggaaagaagaggcctgtagatcagtctcctcaggaaccggactcatcatctggtgttggcaaatcgggcaaacagcctgccagaaaaagactaaatttcggtcagactggcgactcagagtcagtcccagaccctcaacctctcggagaaccaccagcagcccccacaagtttgggatctaatacaatggcttcaggcggtggcgcaccaatggcagacaataacgagggtgccgatggagtgggtaattcctcaggaaattggcattgcgattcccaatggctgggcgacagagtcatcaccaccagcaccagaacctgggccctgcccacttacaacaaccatctctacaagcaaatctccagccaatcaggagcttcaaacgacaaccactactttggctacagcaccccttgggggtattttgactttaacagattccactgccacttctcaccacgtgactggcagcgactcattaacaacaactggggattccggcccaagaaactcagcttcaagctcttcaacatccaagttaaagaggtcacgcagaacgatggcacgacgactattgccaataaccttaccagcacggttcaagtgtttacggactcggagtatcagctcccgtacgtgctcgggtcggcgcaccaaggctgtctcccgccgtttccagcggacgtcttcatggtccctcagtatggatacctcaccctgaacaacggaagtcaagcggtgggacgctcatccttttactgcctggagtacttcccttcgcagatgctaaggactggaaataacttccaattcagctataccttcgaggatgtaccttttcacagcagctacgctcacagccagagtttggatcgcttgatgaatcctcttattgatcagtatctgtactacctgaacagaacgcaaggaacaacctctggaacaaccaaccaatcacggctgctttttagccaggctgggcctcagtctatgtctttgcaggccagaaattggctacctgggccctgctaccggcaacagagactttcaaagactgctaacgacaacaacaacagtaactttccttggacagcggccagcaaatatcatctcaatggccgcgactcgctggtgaatccaggaccagctatggccagtcacaaggacgatgaagaaaaatttttccctatgcacggcaatctaatatttggcaaagaagggacaacggcaagtaacgcagaattagataatgtaatgattacggatgaagaagagattcgtaccaccaatcctgtggcaacagagcagtatggaactgtggcaaataacttgcagagctcaaatacagctcccacgactagaactgtcaatgatcagggggccttacctggcatggtgtggcaagatcgtgacgtgtaccttcaaggacctatctgggcaaagattcctcacacggatggacactttcatccttctcctctgatgggaggctttggactgaaacatccgcctcctcaaatcatgatcaaaaatactccggtaccggcaaatcctccgacgactttcagcccggccaagtttgcttcatttatcactcagtactccactggacaggtcagcgtggaaattgagtgggagctacagaaagaaaacagcaaacgttggaatccagagattcagtacacttccaactacaacaagtctgttaatgtggactttactgtagacactaatggtgtttatagtgaacctcgccctattggaacccggtatctcacacgaaacttgtaa

SEQ ID NO: 23 - DNA, 2172 bp, synthetic construct
  source 1..2172: mol_type = other DNA
atggctgccgatggttatcttccagattggctcgaggacactctctctgaaggaataagacagtggtggaagctcaaacctggcccaccaccaccaaagcccgcagggcggcataaggacgacagcaggggtcttgtgcttcctgggtacaagtacctcggacccttcaacggactcgacaagggagagccggtcaacgaggcagacgccgcggccctcgagcacgacaaagcctacaaccggcagctcgacagcggagacaacccgtacctcaagtacaaccacgccgacgcggagtttcaggagcgccttaaagaagatacgtcttttgggggcaacctcggacgagcagtcttccaggcgaaaaagagggctcttgaacctctgggcctggttgaggaacctgttaaggcccctaccggaaagcggatagacgaccactttccaaaaagaaagaaggcccggaccgaagaggactccaagccttccacctcgtcagacgccgaagctggacccagcggatcccagcagctgcaaatcccagcccaaccagcctcaagtttgggagctgatacaatgtctgcgggaggtggcggcccattgggcgacaataaccaaggtgccgatggagtgggcaatgcctcgggagattggcattgcgattccacgtggatgggggacagagtcgtcaccaagtccacccgaacctgggtgctgcccagctacaacaaccaccagtaccgagagatcaaaagcggctccgtcgacggaagcaacgccaacgcctactttggatacagcaccccctgggggtactttgactttaaccgcttccacagccactggagcccccgagactggcaaagactcatcaacaactactggggcttcagaccccggtccctcagagtcaaaatcttcaacattcaagtcaaagaggtcacggtgcaggactccaccaccaccatcgccaacaacctcacctccaccgtccaagtgtttacggacgacgactaccagctgccctacgtcgtcggcaacgggaccgagggatgcctgccggccttccctccgcaggtctttacgctgccgcagtacggttacgcgacgctgaaccgcgacaacacagaaaatcccaccgagaggagcagcttcttctgcctagagtactttcccagcaagatgctgagaacgggcaacaactttgagtttacctacaactttgaggaggtgcccttccactccagcttcgctcccagtcagaacctcttcaagctggccaacccgctggtggaccagtacttgtaccgcttcgtgagcacaaataacactggcggagtccagttcaacaagaacctggccgggagatacgccaacacctacaaaaactggttcccggggcccatgggccgaacccagggctggaacctgggctccggggtcaaccgcgccagtgtcagcgccttcgccacgaccaataggatggagctcgagggcgcgagttaccaggtgcccccgcagccgaacggcatgaccaacaacctccagggcagcaacacctatgccctggagaacactatgatcttcaacagccagccggcgaacccgggcaccaccgccacgtacctcgagggcaacatgctcatcaccagcgagagcgagacgcagccggtgaaccgcgtggcgtacaacgtcggcgggcagatggccaccaacaaccagagctccaccactgcccccgcgaccggcacgtacaacctccaggaaatcgtgcccggcagcgtgtggatggagagggacgtgtacctccaaggacccatctgggccaagatcccagagacgggggcgcactttcacccctctccggccatgggcggattcggactcaaacacccaccgcccatgatgctcatcaagaacacgcctgtgcccggaaatatcaccagcttctcggacgtgcccgtcagcagcttcatcacccagtacagcaccgggcaggtcaccgtggagatggagtgggagctcaagaaggaaaactccaagaggtggaacccagagatccagtacacaaacaactacaacgacccccagtttgtggactttgccccggacagcaccggggaatacagaaccaccagacctatcggaacccgataccttacccgacccctt

SEQ ID NO: 24 - protein, 200 aa, unidentified
  source 1..200: note = Adeno-associated virus 2
MAADGYLPDWLEDTLSEGIRQWWKLKPGPPPPKPAERHKDDSRGLVLPGYKYLGPFNGLDKGEPVNEADAAALEHDKAYDRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGT

SEQ ID NO: 25 - protein, 200 aa, synthetic construct
MAADGYLPDWLEDTLSEGIRQWWKLKPGPPPPKPAGRHKDDSRGLVLPGYKYLGPFNGLDKGEPVNEADAAALEHDKAYDRQLDSGDNPYLKYNHADAEFQERLKEDTSFGGNLGRAVFQAKKRALEPLGLVEEPVKTAPGKKRPVEHSPVEPDSSSGTGKAGQQPARKRLNFGQTGDADSVPDPQPLGQPPAAPSGLGT

SEQ ID NO: 26 - protein, 200 aa, synthetic construct
MAADGYLPDWLEDTLSEGIRQWWKLKPGPPPPKPAERHKDDSRGLVLPGYKYLGPFNGLDKGEPVNEADAAALEHDKAYNRQLDSGDNPYLKYNHADAEFQERLKEDTSFGGNLGRAVFQAKKRVLEPLGLVEEPVKTAPGKKRPVEHSPVEPDSSSGTGKAGQQPARKRLNFGQTGDADSVPDPQPLGQPPAAPSGLGT

SEQ ID NO: 27 - protein, 200 aa, unidentified
  source 1..200: note = Adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGP

SEQ ID NO: 28 - protein, 200 aa, unidentified
  source 1..200: note = Adeno-associated virus 3
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGP

SEQ ID NO: 29 - protein, 200 aa, unidentified; identical to SEQ ID NO: 28
  source 1..200: note = Adeno-associated virus 5

SEQ ID NO: 30 - protein, 200 aa, unidentified
  source 1..200: note = Adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPFGLVEEGAKTAPGKKRPVEQSPQEPDSSSGIGKTGQQPAKKRLNFGQTGDSESVPDPQPLGEPPATPAAVGP

SEQ ID NO: 31 - protein, 200 aa, unidentified
  source 1..200: note = Adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVG

SEQ ID NO: 32 - protein, 200 aa, unidentified
  source 1..200: note = Adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGS

SEQ ID NO: 33 - protein, 200 aa, unidentified
  source 1..200: note = Adeno-associated virus 10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEAAKTAPGKKRPVEPSPQRSPDSSTGIGKKGQQPAKKRLNFGQTGESESVPDPQPIGEPPAGPSGLG

SEQ ID NO: 34 - protein, 200 aa, unidentified
  source 1..200: note = Adeno-associated virus 12
MAADGYLPDWLEDNLSEGIREWWALKPGAPQPKANQQHQDNGRGLVLPGYKYLGPFNGLDKGEPVNEADAAALEHDKAYDKQLEQGDNPYLKYNHADAEFQQRLATDTSFGGNLGRAVFQAKKRILEPLGLVEEGVKTAPGKKRPLEKTPNRPTNPDSGKAPAKKKQKDGEPADSARRTLDFEDSGAGDGPPEGSSSGEM